(19) United States
(12) Patent Application Publication
(10) Pub. No.: US 2023/0404541 A1
(43) Pub. Date: Dec. 21, 2023

(54) METHOD AND SYSTEM FOR MANAGING ULTRASOUND OPERATIONS USING MACHINE LEARNING AND/OR NON-GUI INTERACTIONS

(71) Applicant: BFLY OPERATIONS, INC., Burlington, MA (US)

(72) Inventors: Brandon Fiegoli, New York, NY (US); Audrey Howell, Guilford, CT (US); Nina Harrison, Bozeman, MT (US); Davinder Ramsingh, Burlington, MD (US); John Martin, Crownsville, MD (US); Dora Fang, Baltimore, MD (US)

(73) Assignee: BFLY OPERATIONS, INC., Burlington, MA (US)

(21) Appl. No.: 18/336,827

(22) Filed: Jun. 16, 2023

Related U.S. Application Data
(60) Provisional application No. 63/413,474, filed on Oct. 5, 2022.
(60) Provisional application No. 63/355,064, filed on Jun. 23, 2022.
(60) Provisional application No. 63/352,889, filed on Jun. 16, 2022.

(51) Int. Cl. A61B 8/00; G16H 10/60; G10L 15/22; A61B 8/08

(52) U.S. Cl. CPC A61B 8/54; G16H 10/60; G10L 15/22; A61B 8/0883; A61B 8/4427; A61B 8/461

(57) ABSTRACT

An ultrasound system may be used for performing an ultrasound imaging exam. The ultrasound system may include an ultrasound imaging device. The ultrasound system may further include a processing device in operative communication with the ultrasound imaging device. The ultrasound system may automatically capture or receive a voice command to capture a clinically relevant ultrasound image.

### CROSS-REFERENCE TO RELATED APPLICATIONS

This United States Provisional Patent Application incorporates herein by reference U.S. Provisional Patent Application Ser. No. 63/352,889, titled “METHOD AND SYSTEM USING NON-GUI INTERACTIONS,” which was filed on Jun. 16, 2022, U.S. Provisional Patent Application Ser. No. 63/355,064, titled “METHOD AND SYSTEM USING NON-GUI INTERACTIONS,” which was filed on Jun. 23, 2022, and U.S. Provisional Patent Application Ser. No. 63/413,474, titled “METHOD AND SYSTEM USING NON-GUI INTERACTIONS AND/OR SIMPLIFIED WORKFLOWS,” which was filed on Oct. 5, 2022.

### BACKGROUND

Imaging technologies are used for multiple purposes. One purpose is to non-invasively diagnose patients. Another purpose is to monitor the performance of medical procedures, such as surgical procedures. Yet another purpose is to monitor post-treatment progress or recovery. Thus, medical imaging technology is used at various stages of medical care. The value of a given medical imaging technology depends on various factors. Such factors include the quality of the images produced, the speed at which the images can be produced, the accessibility of the technology to various types of patients and providers, the potential risks and side effects of the technology to the patient, the impact on patient comfort, and the cost of the technology. The ability to produce three dimensional images is also a consideration for some applications.

### SUMMARY

This summary is provided to introduce a selection of concepts that are further described below in the detailed description. This summary is not intended to identify key or essential features of the claimed subject matter, nor is it intended to be used as an aid in limiting the scope of the claimed subject matter.

In some embodiments, an ultrasound system for performing an ultrasound imaging exam includes an ultrasound imaging device; and a processing device in operative communication with the ultrasound imaging device and configured to perform a method. The method may include initiating an ultrasound imaging application. The method may include receiving a selection of one or more user credentials. The method may include automatically selecting an organization or receive a voice command from a user to select the organization. The method may include automatically selecting a patient or receive a voice command from the user to select the patient. The method may include automatically determining whether a sufficient amount of gel has been applied to the ultrasound imaging device, and upon determining that the sufficient amount of gel has not been applied to the ultrasound imaging device, provide an instruction to the user to apply more gel to the ultrasound imaging device. The method may include automatically selecting or receives a selection of an ultrasound imaging exam type. The method may include automatically select an ultrasound imaging mode or receive a voice command from the user to select the ultrasound imaging mode. The method may include automatically selecting an ultrasound imaging preset or receive a voice command from the user to select the ultrasound imaging preset. The method may include automatically selecting an ultrasound imaging depth or receive a voice command from the user to select the ultrasound imaging depth. The method may include automatically select an ultrasound imaging gain or receive a voice command from the user to select the ultrasound imaging gain. The method may include automatically selecting one or more time gain compensation (TGC) parameters or receive a voice command from the user to select the one or more TGC parameters. The method may include guiding the user to correctly place the ultrasound imaging device in order to capture one or more clinically relevant ultrasound images. The method may include automatically capturing or receive a voice command to capture the one or more clinically relevant ultrasound images. The method may include automatically completing a portion or all of an ultrasound imaging worksheet or receive a voice command from the user to complete the portion or all of the ultrasound imaging worksheet. The method may include associating a signature with the ultrasound imaging exam or request signature of the ultrasound imaging exam later. The method may include automatically uploading the ultrasound imaging exam or receive a voice command from the user to upload the ultrasound imaging exam.

In general, in one aspect, embodiments relate to a method that includes transmitting, using a transducer array, an acoustic signal to an anatomical region of a subject. The method further includes generating ultrasound data based on a reflected signal from the anatomical region in response to transmitting the acoustic signal. The method further includes determining ultrasound angular data using the ultrasound data and various angular bins for a predetermined sector. The method further includes determining a number of predicted B-lines in an ultrasound image using a machine-learning model and the ultrasound angular data. A respective angular bin among the angular bins corresponds to a predetermined sector angle of the ultrasound image. The method further includes determining, in response to determining the number of predicted B-lines, an ultrasound image that identifies the number of predicted B-lines within the ultrasound image.

In general, in one aspect, embodiments relate to a processing device that determines ultrasound angular data using the ultrasound data and various angular bins for a predetermined sector. The processing device further determines a number of predicted B-lines in an ultrasound image using a machine-learning model and the ultrasound angular data. A respective angular bin among the angular bins corresponds to a predetermined sector angle of the ultrasound image. The processing device further determines, in response to determining the number of predicted B-lines, an ultrasound image that identifies the number of predicted B-lines within the ultrasound image.

In general, in one aspect, embodiments relate to ultrasound system for performing an ultrasound imaging exam that includes an ultrasound imaging device and a processing device in operative communication with the ultrasound imaging device. The ultrasound imaging device is configured to transmit, using a transducer array, an acoustic signal to an anatomical region of a subject. The ultrasound imaging device is further configured to generate ultrasound data based on a reflected signal from the anatomical region in response to transmitting the acoustic signal. The processing device is configured to determine ultrasound angular data using the ultrasound data and various angular bins for a predetermined sector. The processing device is further configured to determine a number of predicted B-lines in an ultrasound image using a machine-learning model and the ultrasound angular data. A respective angular bin among the angular bins corresponds to a predetermined sector angle of the ultrasound image. The processing device is further configured to determine, in response to determining the number of predicted B-lines, an ultrasound image that identifies the number of predicted B-lines within the ultrasound image.

In general, in one aspect, embodiments relate to a system that includes a cloud server that includes a first machine-learning model and coupled to a computer network. The system further includes a first ultrasound device that is configured to obtain first non-predicted ultrasound data from a first plurality of subjects. The system further includes a second ultrasound device that is configured to obtain second non-predicted ultrasound data from a second plurality of subjects. The system further includes a first processing system coupled to the first ultrasound device and the cloud server over the computer network. The first processing system is configured to transmit the first non-predicted ultrasound data over the computer network to the cloud server. The system further includes a second processing system coupled to the second ultrasound device and the cloud server over the computer network. The second processing system is configured to transmit the second non-predicted ultrasound data over the computer network to the cloud server. The cloud server is configured to determine a training dataset comprising the first non-predicted ultrasound data and the second non-predicted ultrasound data.

In some embodiments, a diagnosis of a subject is determined based on a number of predicted B-lines. In some embodiments, a predetermined sector corresponds to a middle 30° sector of the ultrasound image, and a predetermined sector angle of a respective angular bin is less than 1° of an ultrasound image. In some embodiments, a machine-learning model outputs a discrete B-line class, a confluent B-line class, and a background data class based on input ultrasound angular data. In some embodiments, a cine is obtained that includes various ultrasound images of an anatomical region. a machine-learning model may be obtained that outputs an image quality score in response to an ultrasound image among the ultrasound images. The ultrasound image may be presented in a graphical user interface on a processing device in response to the image quality score being above the threshold of image quality. The ultrasound image may display a maximum number of B-lines and B-line segmentation data identifying at least one discrete B-lines and at least one confluent B-line. In some embodiments, an ultrasound image is generated based on one or more reflected signals from an anatomical region in response to transmitting one or more acoustic signals. A predicted B-line may be determined using a machine-learning model and the ultrasound image. A determination may be made whether the predicted B-line is a confluent type of B-line using the machine-learning model. A modified ultrasound image may be generated that identifies the predicted B-line within a graphical user interface as being the confluent type of B-line in response to determining that the predicted B-line is the confluent type of B-line.

In some embodiments, first non-predicted ultrasound data and second non-predicted ultrasound data are obtained from various users over a computer network. The first non-predicted ultrasound data and the second non-predicted ultrasound data are obtained using various processing devices coupled to a cloud server over the computer network. A training dataset may be determined that includes the first non-predicted ultrasound data and the second non-predicted ultrasound data. The first non-predicted ultrasound data and the second non-predicted ultrasound data include ultrasound angular data with various labeled B-lines that are identified as being confluent B-lines. First predicted ultrasound data may be generated using an initial model and a first portion of the training dataset in a first machine-learning epoch. The initial model may be a deep neural network that predicts one or more confluent B-lines within an ultrasound image. A determination may be made whether the initial model satisfies a predetermined level of accuracy based on a first comparison between the first predicted ultrasound data and the first non-predicted ultrasound data. The initial model may be updated using a machine-learning algorithm to produce an updated model in response to the initial model failing to satisfy the predetermined level of accuracy.

In some embodiments, a determination is made whether an ultrasound image satisfies an image quality criterion using a machine-learning model. The image quality criterion may correspond to a threshold of image quality that determines whether ultrasound image data can be used to predict a presence of one or more B-lines in the ultrasound image. The ultrasound image may be discarded in response to determining that the ultrasound image fails to satisfy the image quality criterion. In some embodiments, a determination is made whether an ultrasound image satisfies an image quality criterion using a second machine-learning model. The image quality criterion may correspond to a threshold of image quality that determines whether ultrasound image data can be used to predict a presence of one or more B-lines in the ultrasound image. Predicted B-line segmentation data may be determined using a machine-learning model in response to determining that the ultrasound image satisfies the image quality criterion. In some embodiments, a number of B-lines is used to determine pulmonary edema. In some embodiments, a de-identifying process is performed on non-predicted ultrasound data to produce the training dataset. A machine-learning model may be trained using various machine-learning epochs, the training dataset, and a machine-learning algorithm.

In general, in one aspect, embodiments relate to a method that includes transmitting, using a transducer array, one or more acoustic signals to an anatomical region of a subject. The method may include generating ultrasound data based on one or more reflected signals from the anatomical region in response to transmitting the one or more acoustic signals. The method may include determining, by a processor, ultrasound angular data using the ultrasound data and a plurality of angular bins for a predetermined sector. The method may include determining, by the processor, that a predicted B-line is in an ultrasound image using a machine-learning model and the ultrasound angular data. A respective angular bin among various angular bins for the ultrasound angular data corresponds to a predetermined sector angle of the ultrasound image. The method may include determining, by the processing device, whether the predicted B-line is a confluent type of B-line using the machine-learning model. The method may include generating, by the processing device in response to determining that the predicted B-line is the confluent type of B-line, an ultrasound image that identifies the predicted B-line within the ultrasound image as being the confluent type of B-line based on a predicted location of the predicted B-line.

In general, in one aspect, embodiments relate to a method includes transmitting, using a transducer array, a plurality of acoustic signals to an anatomical region of a subject. The method further includes generating a first ultrasound image and a second ultrasound image based on a plurality of reflected signals from the anatomical region in response to transmitting the plurality of acoustic signals. The method further includes determining, by a processor, whether the first ultrasound image satisfies an image quality criterion using a first machine-learning model, wherein the image quality criterion corresponds to a threshold of image quality that determines whether ultrasound image data can be input data for a second machine-learning model that predicts a presence of one or more B-lines. The method further includes discarding, by the processor, the first ultrasound image in response to determining that the first ultrasound image fails to satisfy the image quality criterion. The method further includes determining, by the processor, whether the second ultrasound image satisfies the predetermined criterion using the first machine-learning model. The method further includes determining, by the processor, ultrasound angular data using the second ultrasound image and a plurality of angular bins for a predetermined sector, wherein a respective angular bin among the plurality of angular bins corresponds to a predetermined sector width of the ultrasound image. The method further includes determining, by the processor, a predicted location of a predicted B-line in the ultrasound image using the second machine-learning model. The method further includes adjusting the second ultrasound image to produce a modified ultrasound image that identifies a location of the predicted B-line.

In general, in one aspect, embodiments relate to a method includes obtaining first non-predicted ultrasound data and second non-predicted ultrasound data from a plurality of patients over a computer network. The first non-predicted ultrasound data and the second non-predicted ultrasound data are obtained using various processing devices coupled to a cloud server over the computer network. The method further includes determining a training dataset that includes the first non-predicted ultrasound data and the second non-predicted ultrasound data. The method further includes generating first predicted ultrasound data using a first model and a first portion of the training dataset in a first machine-learning epoch. The initial model is a deep neural network that predicts one or more confluent B-lines within an ultrasound image. The method further includes determining whether the initial model satisfies a predetermined level of accuracy based on a first comparison between the first predicted ultrasound data and the first non-predicted ultrasound data. The method further includes updating the initial model using a machine-learning algorithm to produce an updated model in response to the first model failing to satisfy the predetermined level of accuracy. The method further includes generating, by the processor, second predicted ultrasound data using the updated model and a second portion of the training dataset in a second machine-learning epoch. The method further includes determining whether the updated model satisfies the predetermined level of accuracy based on a second comparison between the second predicted ultrasound data and the second non-predicted ultrasound data. The method further includes generating, by the processor, third predicted ultrasound data for an anatomical region of interest using the updated model and third non-predicted ultrasound data in response to the updated model satisfying the predetermined level of accuracy.

In light of the structure and functions described above, embodiments of the invention may include respective means adapted to carry out various steps and functions defined above in accordance with one or more aspects and any one of the embodiments of one or more aspect described herein.

Other aspects and advantages of the claimed subject matter will be apparent from the following description and the appended claims.

### BRIEF DESCRIPTION OF DRAWINGS

Specific embodiments of the disclosed technology will now be described in detail with reference to the accompanying figures. Like elements in the various figures are denoted by like reference numerals for consistency.

FIG. 1 shows an example system in accordance with one or more embodiments of the technology.

FIGS. 2A, 2B, 3A, 3B, 3C, and 3D shows examples in accordance with one or more embodiments of the technology.

FIG. 4 shows a flowchart in accordance with one or more embodiments of the technology.

FIGS. 5A, 5B, 5C, and 5D show examples in accordance with one or more embodiments of the technology.

FIG. 6 shows a flowchart in accordance with one or more embodiments of the technology.

FIG. 7 shows a schematic block diagram of an example ultrasound system in accordance with one or more embodiments of the technology.

FIG. 8 shows an example handheld ultrasound probe in accordance with one or more embodiments of the technology.

FIG. 9 shows an example patch that includes an example ultrasound probe in accordance with one or more embodiments of the technology.

FIG. 10 shows an example pill that includes an example ultrasound probe in accordance with one or more embodiments of the technology.

FIG. 11 shows a block diagram of an example ultrasound device in accordance with one or more embodiments of the technology.

FIGS. 12 and 13 show flowcharts in accordance with one or more embodiments of the technology.

FIGS. 14 and 15 show examples in accordance with one or more embodiments of the technology.

FIGS. 16A and 16B show flowcharts in accordance with one or more embodiments of the technology.

FIGS. 17A-17Z show examples of a PACE examination in accordance with one or more embodiments of the technology.

FIGS. 18A-18Z and 19A-19I show examples of graphical user interfaces in accordance with one or more embodiments of the technology.

FIGS. 20A-20I show examples of graphical user interfaces associated with some examination workflows in accordance with one or more embodiments of the technology.

### DETAILED DESCRIPTION

In the following detailed description of embodiments of the disclosure, numerous specific details are set forth in order to provide a more thorough understanding of the disclosure. However, it will be apparent to one of ordinary skill in the art that the disclosure may be practiced without these specific details. In other instances, well-known features have not been described in detail to avoid unnecessarily complicating the description.

Throughout the application, ordinal numbers (e.g., first, second, third, etc.) may be used as an adjective for an element (i.e., any noun in the application). The use of ordinal numbers is not to imply or create any particular ordering of the elements nor to limit any element to being only a single element unless expressly disclosed, such as by the use of the terms “before”, “after”, “single”, and other such terminology. Rather, the use of ordinal numbers is to distinguish between the elements. By way of an example, a first element is distinct from a second element, and the first element may encompass more than one element and succeed (or precede) the second element in an ordering of elements.

In general, some embodiments are directed to using machine learning to predict ultrasound data as well as using automated workflows to manage ultrasound operations. In some embodiments, for example, a machine-learning model is used to determine predicted B-line data regarding B-lines in one or more ultrasound operations. B-line data may include B-line segmentations in an image, a particular type of B-line, and other characteristics, such as the number of B-lines in a cine. Likewise, machine learning may also be used to both simplify tasks associated with ultrasound operations, such as provide instructions to an ultrasound device, automatically signing patient reports, and identifying patient information for the subject undergoing an ultrasound analysis.

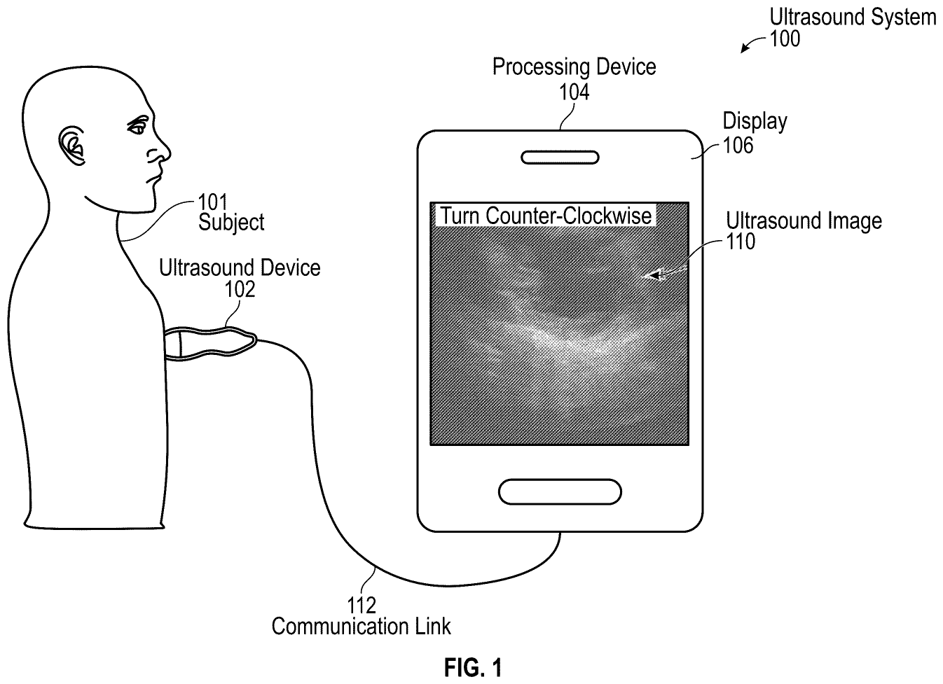

FIG. 1 shows an example ultrasound system 100 including an ultrasound device 102 configured to obtain an ultrasound image of a target anatomical view of a subject 101. As shown, the ultrasound system 100 comprises an ultrasound device 102 that is communicatively coupled to the processing device 104 by a communication link 112. The processing device 104 may be configured to receive ultrasound data from the ultrasound device 102 and use the received ultrasound data to generate an ultrasound image 110 on a display (which may be touch-sensitive) of the processing device 104. In some embodiments, the processing device 104 provides the operator with instructions (e.g., images, videos, or text) prior to the operator scanning the subject 101. The processing device 104 may provide quality indicators and/or labels of anatomical features during scanning of the subject 101 to assist a user in collecting clinically relevant ultrasound images.

The ultrasound device 102 may be configured to generate ultrasound data. The ultrasound device 102 may be configured to generate ultrasound data by, for example, emitting acoustic waves into the subject 101 and detecting the reflected acoustic waves. The detected reflected acoustic wave may be analyzed to identify various properties of the tissues through which the acoustic wave traveled, such as a density of the tissue. The ultrasound device 102 may be implemented in any of a variety of ways. For example, the ultrasound device 102 may be implemented as a handheld device (as shown in FIG. 1) or as a patch that is coupled to patient using, for example, an adhesive.

The ultrasound device 102 may transmit ultrasound data to the processing device 104 using the communication link 112. The communication link 112 may be a wired or wireless communication link. In some embodiments, the communication link 112 may be implemented as a cable such as a Universal Serial Bus (USB) cable or a Lightning cable. In these embodiments, the cable may also be used to transfer power from the processing device 104 to the ultrasound device 102. In other embodiments, the communication link 112 may be a wireless communication link such as a BLUETOOTH, WiFi, or ZIGBEE wireless communication link.

The processing device 104 may comprise one or more processing elements (such as a processor) to, for example, process ultrasound data received from the ultrasound device 102. Additionally, the processing device 104 may comprise one or more storage elements (such as a non-transitory computer readable medium) to, for example, store instructions that may be executed by the processing element(s) and/or store all or any portion of the ultrasound data received from the ultrasound device 102. It should be appreciated that the processing device 104 may be implemented in any of a variety of ways. For example, the processing device 104 may be implemented as a mobile device (e.g., a mobile smartphone, a tablet, or a laptop) with an integrated display 106 as shown in FIG. 1. In other examples, the processing device 104 may be implemented as a stationary device such as a desktop computer.

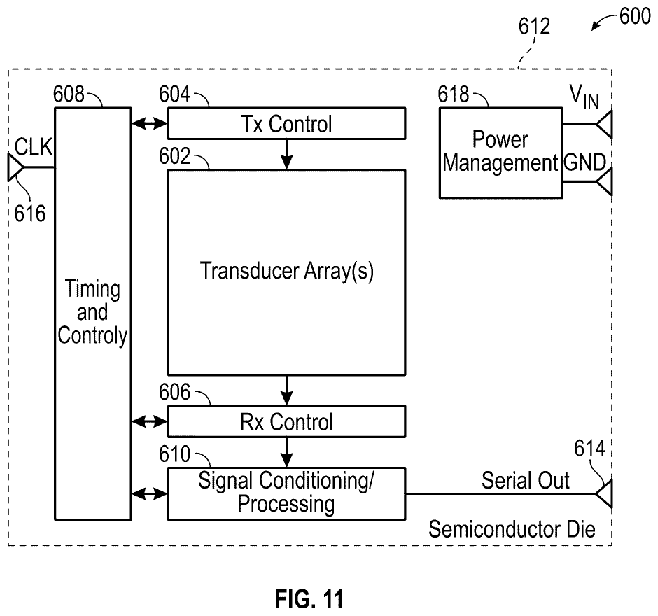

FIG. 11 is a block diagram of an example of an ultrasound device in accordance with some embodiments of the technology described herein. The illustrated ultrasound device 600 may include one or more ultrasonic transducer arrangements (e.g., arrays) 602, transmit (TX) circuitry 604, receive (RX) circuitry 606, a timing and control circuit 608, a signal conditioning/processing circuit 610, and/or a power management circuit 618.

The one or more ultrasonic transducer arrays 602 may take on any of numerous forms, and aspects of the present technology do not necessarily require the use of any particular type or arrangement of ultrasonic transducer cells or ultrasonic transducer elements. For example, multiple ultrasonic transducer elements in the ultrasonic transducer array 602 may be arranged in one-dimension, or two-dimensions. Although the term “array” is used in this description, it should be appreciated that in some embodiments the ultrasonic transducer elements may be organized in a non-array fashion. In various embodiments, each of the ultrasonic transducer elements in the array 602 may, for example, include one or more capacitive micromachined ultrasonic transducers (CMUTs), or one or more piezoelectric micromachined ultrasonic transducers (PMUTs).

In a non-limiting example, the ultrasonic transducer array 602 may include between approximately 6,000-10,000 (e.g., 8,960) active CMUTs on the chip, forming an array of hundreds of CMUTs by tens of CMUTs (e.g., 140×64). The CMUT element pitch may be between 150-250 um, such as 208 um, and thus, result in the total dimension of between 10-50 mm by 10-50 mm (e.g., 29.12 mm×13.312 mm).

In some embodiments, the TX circuitry 604 may, for example, generate pulses that drive the individual elements of, or one or more groups of elements within, the ultrasonic transducer array(s) 602 so as to generate acoustic signals to be used for imaging. The RX circuitry 606, on the other hand, may receive and process electronic signals generated by the individual elements of the ultrasonic transducer array(s) 602 when acoustic signals impinge upon such elements.

With further reference to FIG. 11, in some embodiments, the timing and control circuit 608 may be, for example, responsible for generating all timing and control signals that are used to synchronize and coordinate the operation of the other elements in the device 600. In the example shown, the timing and control circuit 608 is driven by a single clock signal CLK supplied to an input port 616. The clock signal CLK may be, for example, a high-frequency clock used to drive one or more of the on-chip circuit components. In some embodiments, the clock signal CLK may, for example, be a 1.5625 GHz or 2.5 GHz clock used to drive a high-speed serial output device (not shown in FIG. 11) in the signal conditioning/processing circuit 610, or a 20 Mhz or 40 MHz clock used to drive other digital components on the die 612, and the timing and control circuit 608 may divide or multiply the clock CLK, as necessary, to drive other components on the die 612. In other embodiments, two or more clocks of different frequencies (such as those referenced above) may be separately supplied to the timing and control circuit 608 from an off-chip source.

In some embodiments, the output range of a same (or single) transducer unit in an ultrasound device may be anywhere in a range of 1-12 MHz (including the entire frequency range from 1-12 MHz), making it a universal solution, in which there is no need to change the ultrasound heads or units for different operating ranges or to image at different depths within a patient. That is, the transmit and/or receive frequency of the transducers of the ultrasonic transducer array may be selected to be any frequency or range of frequencies within the range of 1 MHz-12 MHz. The universal device 600 described herein may thus be used for a broad range of medical imaging tasks including, but not limited to, imaging a patient's liver, kidney, heart, bladder, thyroid, carotid artery, lower venous extremity, and performing central line placement. Multiple conventional ultrasound probes would have to be used to perform all these imaging tasks. By contrast, a single universal ultrasound device 600 may be used to perform all these tasks by operating, for each task, at a frequency range appropriate for the task, as shown in the examples of Table 1 together with corresponding depths at which the subject may be imaged.

TABLE 1
Illustrative depths and frequencies at which an ultrasound
device implemented in accordance with embodiments
described herein may image a subject.

| Organ | Frequencies | | Depth (up to) | |
|---|---|---|---|---|
| Liver/Right Kidney | 2-5 | MHz | 15-20 | cm |
| Cardiac (adult) | 1-5 | MHz | 20 | cm |
| Bladder | 2-5 MHz; 3-6 MHz | | 10-15 cm; 5-10 cm | |
| Lower extremity venous | 4-7 | MHz | 4-6 | cm |
| Thyroid | 7-12 | MHz | 4 | cm |
| Carotid | 5-10 | MHz | 4 | cm |
| Central Line Placement | 5-10 | MHz | 4 | cm |

The power management circuit 618 may be, for example, responsible for converting one or more input voltages VIN from an off-chip source into voltages needed to carry out operation of the chip, and for otherwise managing power consumption within the device 600. In some embodiments, for example, a single voltage (e.g., 12V, 80V, 100V, 120V, etc.) may be supplied to the chip and the power management circuit 618 may step that voltage up or down, as necessary, using a charge pump circuit or via some other DC-to-DC voltage conversion mechanism. In other embodiments, multiple different voltages may be supplied separately to the power management circuit 618 for processing and/or distribution to the other on-chip components.

In the embodiment shown above, all of the illustrated elements are formed on a single semiconductor die 612. It should be appreciated, however, that in alternative embodiments one or more of the illustrated elements may be instead located off-chip, in a separate semiconductor die, or in a separate device. Alternatively, one or more of these components may be implemented in a DSP chip, a field programmable gate array (FPGA) in a separate chip, or a separate application specific integrated circuitry (ASIC) chip. Additionally, and/or alternatively, one or more of the components in the beamformer may be implemented in the semiconductor die 612, whereas other components in the beamformer may be implemented in an external processing device in hardware or software, where the external processing device is capable of communicating with the ultrasound device 600.

In addition, although the illustrated example shows both TX circuitry 604 and RX circuitry 606, in alternative embodiments only TX circuitry or only RX circuitry may be employed. For example, such embodiments may be employed in a circumstance where one or more transmission-only devices are used to transmit acoustic signals and one or more reception-only devices are used to receive acoustic signals that have been transmitted through or reflected off of a subject being ultrasonically imaged.

It should be appreciated that communication between one or more of the illustrated components may be performed in any of numerous ways. In some embodiments, for example, one or more high-speed busses (not shown), such as that employed by a unified Northbridge, may be used to allow high-speed intra-chip communication or communication with one or more off-chip components.

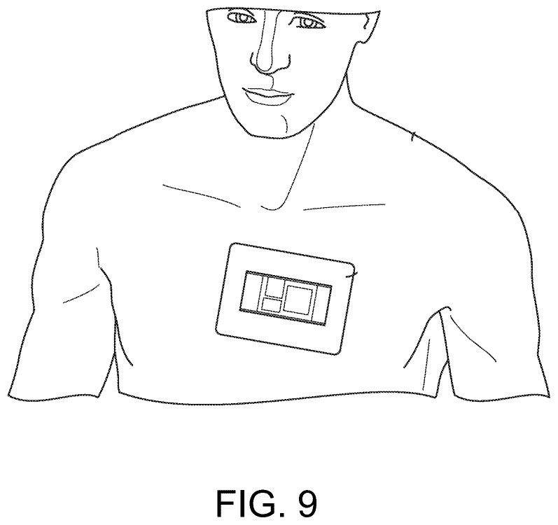

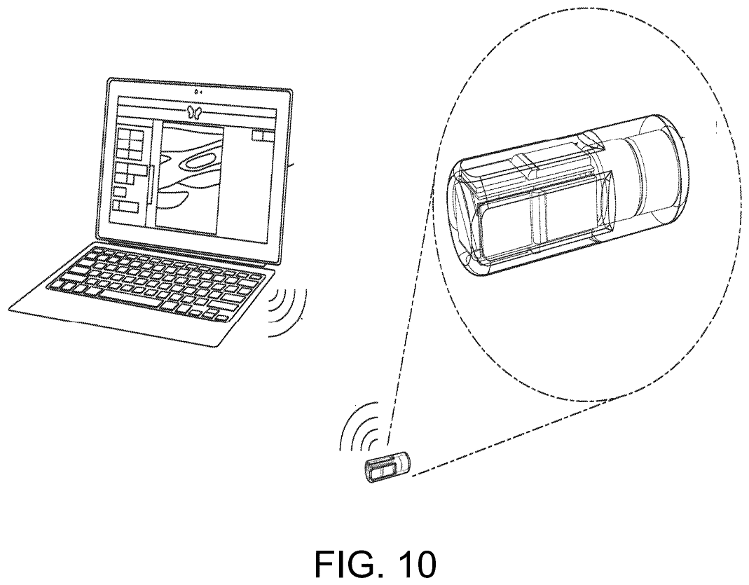

In some embodiments, the ultrasonic transducer elements of the ultrasonic transducer array 602 may be formed on the same chip as the electronics of the TX circuitry 604 and/or RX circuitry 606. The ultrasonic transducer arrays 602, TX circuitry 604, and RX circuitry 606 may be, in some embodiments, integrated in a single ultrasound probe. In some embodiments, the single ultrasound probe may be a hand-held probe including, but not limited to, the hand-held probes described below with reference to FIG. 8. In other embodiments, the single ultrasound probe may be embodied in a patch that may be coupled to a patient. FIG. 9 provides a non-limiting illustration of such a patch. The patch may be configured to transmit, wirelessly, data collected by the patch to one or more external devices for further processing. In other embodiments, the single ultrasound probe may be embodied in a pill that may be swallowed by a patient. The pill may be configured to transmit, wirelessly, data collected by the ultrasound probe within the pill to one or more external devices for further processing. FIG. 10 illustrates a non-limiting example of such a pill.

A CMUT may include, for example, a cavity formed in a CMOS wafer, with a membrane overlying the cavity, and in some embodiments sealing the cavity. Electrodes may be provided to create an ultrasonic transducer cell from the covered cavity structure. The CMOS wafer may include integrated circuitry to which the ultrasonic transducer cell may be connected. The ultrasonic transducer cell and CMOS wafer may be monolithically integrated, thus forming an integrated ultrasonic transducer cell and integrated circuit on a single substrate (the CMOS wafer).

In the example shown, one or more output ports 614 may output a high-speed serial data stream generated by one or more components of the signal conditioning/processing circuit 610. Such data streams may be, for example, generated by one or more USB 3.0 modules, and/or one or more 10 GB, 40 GB, or 100 GB Ethernet modules, integrated on the die 612. It is appreciated that other communication protocols may be used for the output ports 614.

In some embodiments, the signal stream produced on output port 614 can be provided to a computer, tablet, or smartphone for the generation and/or display of two-dimensional, three-dimensional, and/or tomographic images. In some embodiments, the signal provided at the output port 614 may be ultrasound data provided by the one or more beamformer components or auto-correlation approximation circuitry, where the ultrasound data may be used by the computer (external to the ultrasound device) for displaying the ultrasound images. In embodiments in which image formation capabilities are incorporated in the signal conditioning/processing circuit 610, even relatively low-power devices, such as smartphones or tablets which have only a limited amount of processing power and memory available for application execution, can display images using only a serial data stream from the output port 614. As noted above, the use of on-chip analog-to-digital conversion and a high-speed serial data link to offload a digital data stream is one of the features that helps facilitate an “ultrasound on a chip” solution according to some embodiments of the technology described herein.

Devices 600 such as that shown in FIG. 11 may be used in various imaging and/or treatment (e.g., HIFU) applications, and the particular examples described herein should not be viewed as limiting. In one illustrative implementation, for example, an imaging device including an N×M planar or substantially planar array of CMUT elements may itself be used to acquire an ultrasound image of a subject (e.g., a person's abdomen) by energizing some or all of the elements in the ultrasonic transducer array(s) 602 (either together or individually) during one or more transmit phases, and receiving and processing signals generated by some or all of the elements in the ultrasonic transducer array(s) 602 during one or more receive phases, such that during each receive phase the CMUT elements sense acoustic signals reflected by the subject. In other implementations, some of the elements in the ultrasonic transducer array(s) 602 may be used only to transmit acoustic signals and other elements in the same ultrasonic transducer array(s) 602 may be simultaneously used only to receive acoustic signals. Moreover, in some implementations, a single imaging device may include a P×Q array of individual devices, or a P×Q array of individual N×M planar arrays of CMUT elements, which components can be operated in parallel, sequentially, or according to some other timing scheme so as to allow data to be accumulated from a larger number of CMUT elements than can be embodied in a single device 600 or on a single die 612.

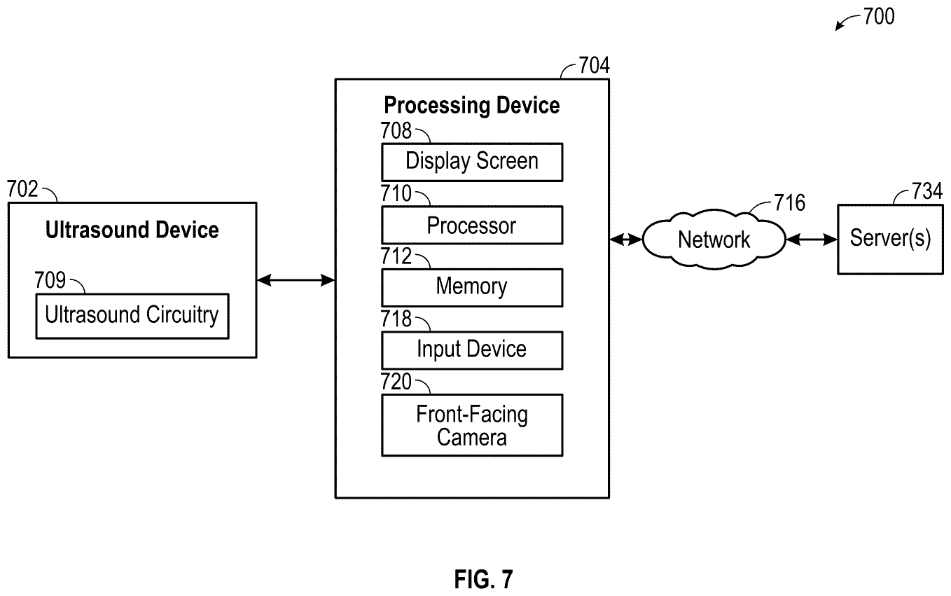

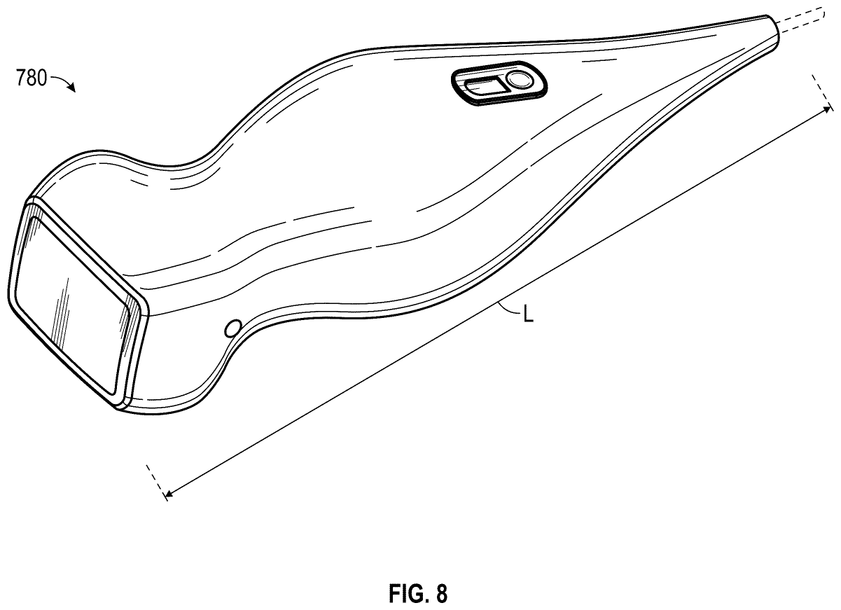

FIG. 7 illustrates a schematic block diagram of an example ultrasound system 700 which may implement various aspects of the technology described herein. In some embodiments, ultrasound system 700 may include an ultrasound device 702, an example of which is implemented in ultrasound device 600. For example, the ultrasound device 702 may be a handheld ultrasound probe. Additionally, the ultrasound system 700 may include a processing device 704, a communication network 716, and one or more servers 734. The ultrasound device 702 may be configured to generate ultrasound data that may be employed to generate an ultrasound image. The ultrasound device 702 may be constructed in any of a variety of ways. In some embodiments, the ultrasound device 702 includes a transmitter that transmits a signal to a transmit beamformer which in turn drives transducer elements within a transducer array to emit pulsed ultrasound signals into a structure, such as a patient. The pulsed ultrasound signals may be back-scattered from structures in the body, such as blood cells or muscular tissue, to produce echoes that return to the transducer elements. These echoes may then be converted into electrical signals by the transducer elements and the electrical signals are received by a receiver. The electrical signals representing the received echoes are sent to a receive beamformer that outputs ultrasound data. In some embodiments, the ultrasound device 702 may include an ultrasound circuitry 709 that may be configured to generate the ultrasound data. For example, the ultrasound device 702 may include semiconductor die 612 for implementing the various techniques described in.

Reference is now made to the processing device 704. In some embodiments, the processing device 704 may be communicatively coupled to the ultrasound device 702 (e.g., 102 in FIG. 1) wirelessly or in a wired fashion (e.g., by a detachable cord or cable) to implement at least a portion of the process for approximating the auto-correlation of ultrasound signals. For example, one or more beamformer components may be implemented on the processing device 704. In some embodiments, the processing device 704 may include one or more processing devices (processors) 710, which may include specially-programmed and/or special-purpose hardware such as an ASIC chip. The processor 710 may include one or more graphics processing units (GPUs) and/or one or more tensor processing units (TPUs). TPUs may be ASICs specifically designed for machine learning (e.g., deep learning). The TPUs may be employed to, for example, accelerate the inference phase of a neural network.

In some embodiments, the processing device 704 may be configured to process the ultrasound data received from the ultrasound device 702 to generate ultrasound images for display on the display screen 708. The processing may be performed by, for example, the processor(s) 710. The processor(s) 710 may also be adapted to control the acquisition of ultrasound data with the ultrasound device 702. The ultrasound data may be processed in real-time during a scanning session as the echo signals are received. In some embodiments, the displayed ultrasound image may be updated at a rate of at least 5 Hz, at least 10 Hz, at least 20 Hz, at a rate between 5 and 60 Hz, at a rate of more than 20 Hz. For example, ultrasound data may be acquired even as images are being generated based on previously acquired data and while a live ultrasound image is being displayed. As additional ultrasound data is acquired, additional frames or images generated from more-recently acquired ultrasound data are sequentially displayed. Additionally, or alternatively, the ultrasound data may be stored temporarily in a buffer during a scanning session and processed in less than real-time.

In some embodiments, the processing device 704 may be configured to perform various ultrasound operations using the processor(s) 710 (e.g., one or more computer hardware processors) and one or more articles of manufacture that include non-transitory computer-readable storage media such as the memory 712. The processor(s) 710 may control writing data to and reading data from the memory 712 in any suitable manner. To perform certain of the processes described herein, the processor(s) 710 may execute one or more processor-executable instructions stored in one or more non-transitory computer-readable storage media (e.g., the memory 712), which may serve as non-transitory computer-readable storage media storing processor-executable instructions for execution by the processor(s) 710.

The camera 720 may be configured to detect light (e.g., visible light) to form an image. The camera 720 may be on the same face of the processing device 704 as the display screen 708. The display screen 708 may be configured to display images and/or videos, and may be, for example, a liquid crystal display (LCD), a plasma display, and/or an organic light emitting diode (OLED) display on the processing device 704. The input device 718 may include one or more devices capable of receiving input from a user and transmitting the input to the processor(s) 710. For example, the input device 718 may include a keyboard, a mouse, a microphone, touch-enabled sensors on the display screen 708, and/or a microphone. The display screen 708, the input device 718, the camera 720, and/or other input/output interfaces (e.g., speaker) may be communicatively coupled to the processor(s) 710 and/or under the control of the processor 710.

It should be appreciated that the processing device 704 may be implemented in any of a variety of ways. For example, the processing device 704 may be implemented as a handheld device such as a mobile smartphone or a tablet. Thereby, a user of the ultrasound device 702 may be able to operate the ultrasound device 702 with one hand and hold the processing device 704 with another hand. In other examples, the processing device 704 may be implemented as a portable device that is not a handheld device, such as a laptop. In yet other examples, the processing device 704 may be implemented as a stationary device such as a desktop computer. The processing device 704 may be connected to the network 716 over a wired connection (e.g., via an Ethernet cable) and/or a wireless connection (e.g., over a WiFi network). The processing device 704 may thereby communicate with (e.g., transmit data to or receive data from) the one or more servers 734 over the network 716. For example, a party may provide from the server 734 to the processing device 704 processor-executable instructions for storing in one or more non-transitory computer-readable storage media (e.g., the memory 712) which, when executed, may cause the processing device 704 to perform ultrasound processes. FIG. 7 should be understood to be non-limiting. For example, the ultrasound system 700 may include fewer or more components than shown and the processing device 704 and ultrasound device 702 may include fewer or more components than shown. In some embodiments, the processing device 704 may be part of the ultrasound device 702.

FIG. 8 illustrates an example handheld ultrasound probe, in accordance with certain embodiments described herein. The handheld ultrasound probe 780 may implement any of the ultrasound imaging devices described herein. The handheld ultrasound probe 780 may have a suitable dimension and weight. For example, the ultrasound probe 780 may have a cable for wired communication with a processing device, and have a length L about 100 mm-300 mm (e.g., 175 mm) and a weight about 200 grams-500 grams (e.g., 312 g). In another example, the ultrasound probe 780 may be capable of communicating with a processing device wirelessly. As such, the handheld ultrasound probe 780 may have a length about 140 mm and a weight about 265 g. It is appreciated that other dimensions and weight may be possible.

Further description of ultrasound devices and systems may be found in U.S. Pat. No. 9,521,991, the content of which is incorporated by reference herein in its entirety; and U.S. Pat. No. 11,311,274, the content of which is incorporated by reference herein in its entirety.

Turning to machine learning, devices and systems may include hardware and/or software with functionality for generating and/or updating one or more machine-learning models to determined predicted ultrasound data, such as predicted B-lines. Examples of machine-learning models may include random forest models and artificial neural networks, such as convolutional neural networks, deep neural networks, and recurrent neural networks. Machine-learning (ML) models may also include support vector machines (SVMs), Naïve Bayes models, ridge classifier models, gradient boosting models, decision trees, inductive learning models, deductive learning models, supervised learning models, unsupervised learning models, reinforcement learning models, and the like. In a deep neural network, for example, a layer of neurons may be trained on a predetermined list of features based on the previous network layer's output. Thus, as data progresses through the deep neural network, more complex features may be identified within the data by neurons in later layers. Likewise, a U-net model or other type of convolutional neural network model may include various convolutional layers, pooling layers, fully connected layers, and/or normalization layers to produce a particular type of output. Thus, convolution and pooling functions may be the activation functions within a convolutional neural network. In some embodiments, two or more different types of machine-learning models are integrated into a single machine-learning architecture, e.g., a machine-learning model may include a random forest model and various neural networks. In some embodiments, a remote server may generate augmented data or synthetic data to produce a large amount of interpreted data for training a particular model.

In some embodiments, various types of machine-learning algorithms may be used to train the model, such as a backpropagation algorithm. In a backpropagation algorithm, gradients are computed for each hidden layer of a neural network in reverse from the layer closest to the output layer proceeding to the layer closest to the input layer. As such, a gradient may be calculated using the transpose of the weights of a respective hidden layer based on an error function (also called a “loss function”). The error function may be based on various criteria, such as mean squared error function, a similarity function, etc., where the error function may be used as a feedback mechanism for tuning weights in the machine-learning model.

In some embodiments, a machine-learning model is trained using multiple epochs. For example, an epoch may be an iteration of a model through a portion or all of a training dataset. As such, a single machine-learning epoch may correspond to a specific batch of training data, where the training data is divided into multiple batches for multiple epochs. Thus, a machine-learning model may be trained iteratively using epochs until the model achieves a predetermined criterion, such as predetermined level of prediction accuracy or training over a specific number of machine-learning epochs or iterations. Thus, better training of a model may lead to better predictions by a trained model.

With respect to artificial neural networks, for example, an artificial neural network may include one or more hidden layers, where a hidden layer includes one or more neurons. A neuron may be a modelling node or object that is loosely patterned on a neuron of the human brain. In particular, a neuron may combine data inputs with a set of coefficients, i.e., a set of network weights for adjusting the data inputs. These network weights may amplify or reduce the value of a particular data input, thereby assigning an amount of significance to various data inputs for a task being modeled. Through machine learning, a neural network may determine which data inputs should receive greater priority in determining one or more specified outputs of the artificial neural network. Likewise, these weighted data inputs may be summed such that this sum is communicated through a neuron's activation function to other hidden layers within the artificial neural network. As such, the activation function may determine whether and to what extent an output of a neuron progresses to other neurons where the output may be weighted again for use as an input to the next hidden layer.

Turning to recurrent neural networks, a recurrent neural network (RNN) may perform a particular task repeatedly for multiple data elements in an input sequence (e.g., a sequence of temperature values or flow rate values), with the output of the recurrent neural network being dependent on past computations. As such, a recurrent neural network may operate with a memory or hidden cell state, which provides information for use by the current cell computation with respect to the current data input. For example, a recurrent neural network may resemble a chain-like structure of RNN cells, where different types of recurrent neural networks may have different types of repeating RNN cells. Likewise, the input sequence may be time-series data, where hidden cell states may have different values at different time steps during a prediction or training operation. For example, where a deep neural network may use different parameters at each hidden layer, a recurrent neural network may have common parameters in an RNN cell, which may be performed across multiple time steps. To train a recurrent neural network, a supervised learning algorithm such as a backpropagation algorithm may also be used. In some embodiments, the backpropagation algorithm is a backpropagation through time (BPTT) algorithm. Likewise, a BPTT algorithm may determine gradients to update various hidden layers and neurons within a recurrent neural network in a similar manner as used to train various deep neural networks.

Embodiments are contemplated with different types of RNNs. For example, classic RNNs, long short-term memory (LSTM) networks, a gated recurrent unit (GRU), a stacked LSTM that includes multiple hidden LSTM layers (i.e., each LSTM layer includes multiple RNN cells), recurrent neural networks with attention (i.e., the machine-learning model may focus attention on specific elements in an input sequence), bidirectional recurrent neural networks (e.g., a machine-learning model that may be trained in both time directions simultaneously, with separate hidden layers, such as forward layers and backward layers), as well as multidimensional LSTM networks, graph recurrent neural networks, grid recurrent neural networks, etc. With regard to LSTM networks, an LSTM cell may include various output lines that carry vectors of information, e.g., from the output of one LSTM cell to the input of another LSTM cell. Thus, an LSTM cell may include multiple hidden layers as well as various pointwise operation units that perform computations such as vector addition.

In some embodiments, a server uses one or more ensemble learning methods to produce a hybrid-model architecture. For example, an ensemble learning method may use multiple types of machine-learning models to obtain better predictive performance than available with a single machine-learning model. In some embodiments, for example, an ensemble architecture may combine multiple base models to produce a single machine-learning model. One example of an ensemble learning method is a BAGGing model (i.e., BAGGing refers to a model that performs Bootstrapping and Aggregation operations) that combines predictions from multiple neural networks to add a bias that reduces variance of a single trained neural network model. Another ensemble learning method includes a stacking method, which may involve fitting many different model types on the same data and using another machine-learning model to combine various predictions.

Turning to random forests, a random forest model may an algorithmic model that combines the output of multiple decision trees to reach a single predicted result. For example, a random forest model may be composed of a collection of decision trees, where training the random forest model may be based on three main hyperparameters that include node size, a number of decision trees, and a number of input features being sampled. During training, a random forest model may allow different decision trees to randomly sample from a dataset with replacement (e.g., from a bootstrap sample) to produce multiple final decision trees in the trained model. For example, when multiple decision trees form an ensemble in the random forest model, this ensemble may determine more accurate predicted data, particularly when the individual trees are uncorrelated with each other.

In some embodiments, a machine-learning model is disposed on-board a processing device. For example, a specific hardware accelerator and/or an embedded system may be implemented to perform inference operations based on ultrasound data and/or other data. Likewise, sparse coding and sparse machine-learning models may be used to reduce the necessary computational resources to implement a machine-learning model on the processing device for an ultrasound system. A sparse machine-learning model may include a model that is gradually reduced in size (e.g., reducing number of hidden layers, neurons, etc.) until the model achieves a predetermined degree of accuracy for inference operations, such as predicting B-lines, and also computing size sufficient for operating on a processing device.

### Predicting B-Line Data Using Machine Learning

Some embodiments relate to a B-line counting method that automatically determines a number of predicted B-lines present within an ultrasound image of an anatomical region of a subject. For example, the number of B-lines in a rib space may be determined while scanning with a Lung preset (i.e., an abdomen imaging setting optimized for lung ultrasound). After noting individual B-lines within ultrasound image data, the maximum number of B-lines may be determined in an intercostal space at a particular moment (e.g., one frame in a cine that is a sequence of ultrasound images). A B-line may refer to a hyperechoic artifact that may be relevant for a particular diagnosis in lung ultrasonography. For example, a B-line may exhibit one or more features within an ultrasound image, such as a comet-tail, arising from a pleural line, being well-defined, extending indefinitely, erasing A-lines, and/or moving in concert with lung sliding, if lung sliding is present. Moreover, a B-line may be a discrete B-line or a confluent B-line. A discrete B-line may be a single B-line disposed within a single angular bin. For angular bins, an ultrasound image may be divided into a predetermined number of sectors with specific widths (e.g., a 70° ultrasound image may have 100 angular bins that span the full width of the 70° sector). On the other hand, a confluent B-line may correspond to two or more adjacent discrete B-lines located across multiple angular bins within an ultrasound image.

By determining and analyzing B-lines for a living subject, the status of the subject may be determined for both acute and chronic disease management. However, some previous methods of measuring lung wetness via B-line counting are highly susceptible to inter-observer variability difference, such that different clinicians may determine different numbers and/or types of B-lines within an ultrasound image. In contrast, some embodiments can provide an automated B-line counting that provides faster lung assessment in urgent situations and consistent methods for long-term patient monitoring. During operation, the user may position a transducer array in an anatomical space, such as a rib space, to analyze a lung region. A processing device may examine a predetermined sector, such as a central 30° sector, in each frame with an internal quality check to determine whether obtained ultrasound data is appropriate for displaying B-lines overlays. If a processing device deems the input image to be appropriate, B-line segmentation data may overlay live B-line annotations on top of the image. Discrete B-lines may be represented with single lines and confluent B-lines may be represented with bracketed lines enclosing an image region.

Using one or more machine-learnings models, a B-line may be predicted among a set of individual or contiguous angular bins through input ultrasound data (e.g., respective ultrasound image data associated with respective angular bins) that represent the presence of a particular B-line. Thus, a B-line segmentation may include an overlay on an ultrasound image to denote the location of any predicted B-lines. Moreover, this predicted location may be based on the centroid of the contiguous angular bins. In some embodiments, one or more predicted B-lines are determined using a deep neural network. For example, a machine-learning model may be trained using annotations or labels assigned by a human analysist to a cine, image, or a region of an image to train the model. Furthermore, some embodiments may include a method that determines a number of discrete B-lines and, afterwards, determines a count of one or more confluent B-lines as the percentage of the anatomical region filled with confluent B-lines divided by a predetermined number, such as 10. For example, if 40% of a rib space is filled with B-lines, then the count may be 4. As such, the B-line count in a particular cine frame may include confluent B-lines and discrete B-lines added together.

In some embodiments, B-line filtering is performed on ultrasound angular data. Using bin voting in a machine-learning model, for example, if the background votes exceed the number of confluent or discrete vote, an angular bin may be counted as a background bin. On the other hand, if the number of discrete votes exceeds the number of confluent votes, the angular bin is counted as a discrete bin. In order to clean up some of the edge cases generated by a bin voting process, various filtering steps may be applied serially using various voting rules after voting is performed. One voting rule may require that any discrete bins that are adjacent to confluent bins are converted to confluent bins. Another voting rule may be applied iteratively where any continuous run of discrete bins that are larger than a predetermined number of bins (e.g., 20 bins) may be converted to confluent bins. Another voting rule may require that any continuous run of discrete bins that are smaller than a predetermined number (e.g., 3 bins) are converted to background bins. Finally, any continuous run of confluent bins that are smaller than a predetermined number of bins (e.g., 7 bins) are converted to background bins.

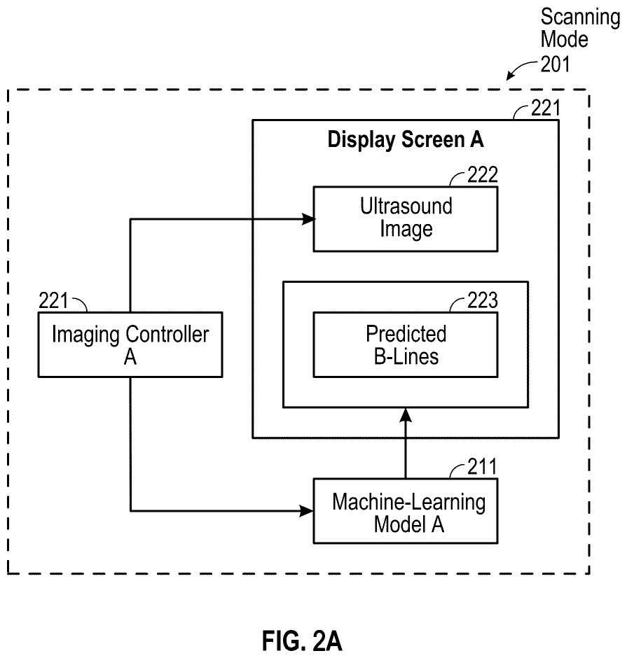

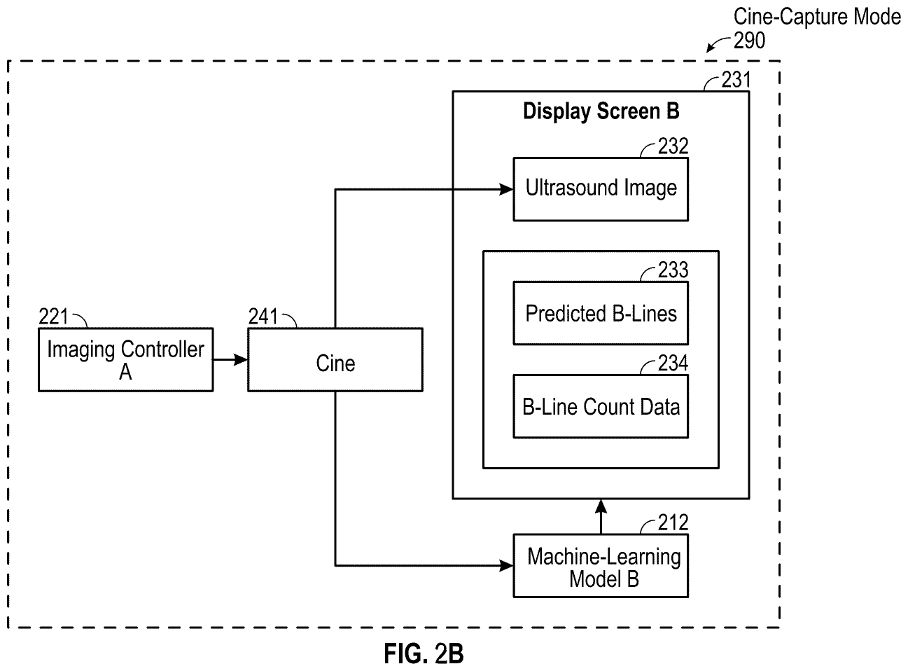

Turning to FIGS. 2A and 2B, FIGS. 2A and 2B show example systems in accordance with one or more embodiments. In FIG. 2A, a system is illustrated for performing a scanning mode 201 where a display screen A 221 shows a scanning mode user interface. The display screen A 221 may present various ultrasound images (e.g., ultrasound image 232) and predicted B-lines 223 that are determined using a machine-learning model A 211. The predicted B-lines 223 may include B-line segmentation that are predicted in real-time while a user is operating an ultrasound device. As shown, an imaging controller may include hardware and/or software that is included in a processing device as described above in FIGS. 1 and/or 7-11 and the accompanying description. The imaging controller may manage and/or use ultrasound image data that comes from an ultrasound device as inputs to a machine-learning workflow. For example, the imaging controller may present information, such as identified B-lines, on top of one or more ultrasound images. The imaging controller may receive a raw imaging signal that is transmitted from an ultrasound device to a processing device that includes the imaging controller. At the processing device, ultrasound image data may be decoded and processed before being presented to the user performing a scanning operation.

In FIG. 2B, a system is illustrated performing a cine-capture mode 290 where a display screen B 231 presents a cine count screen user interface. As part of a cine-capture mode 290, a cine with a predetermined length (e.g., 6 seconds that captures a respiratory cycle) may be recorded and fed into the machine-learning model B 212. After the analysis is done, the recorded cine may be presented on display screen B 231 and overlaid with the results from the machine-learning model B 212. Furthermore, the display screen B 231 may present ultrasound images and predicted B-lines 233 with B-line count data 234 for a recorded cine 241 (e.g., a cine of 6 second). The imaging controller may overlay ultrasound images (i.e., individual frames of the cine) with the locations of any predicted B-lines determined by machine-learning model B 212. In addition, the maximum B-line count among multiple frames of the cine may be presented to a user among the B-line count data 234. On the other hand, if no B-lines are among any ultrasound images in the cine, no result may be provided to the user. A user of a processing device may be able to save, upload, and/or store the captured cine (with overlaid B-line count data 234).

In some embodiments, a processing device and/or a remote server include one or more inference engines that are used to feed image data to input layers of one or more machine-learning models. The inference engine may obtain as inputs one or more ultrasound images and associated metadata about the images as well as various transducer state information. The inference engine may then return the predicted outputs produced by the machine-learning model. When an automated B-line counter is selected by a user on a processing device, the inference engine may be initiated with the machine-learning model. Furthermore, one or more machine learning models may use deep learning to analyze various ultrasound images, such as lung images, for the presence of B-lines. As such, a machine-learning model may include a deep neural network with two or more submodels that accomplish different functions in response to an input ultrasound image or frame. One submodel may identify the presence of B-lines thereby indicating the predicted locations of the B-lines within a B-mode image. Another submodel may determine the suitability of an image or frame for identifying the presence B-lines.

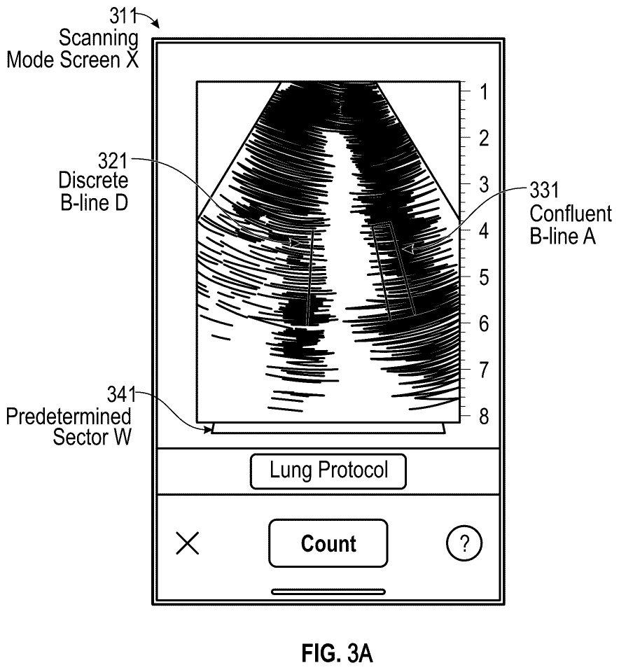

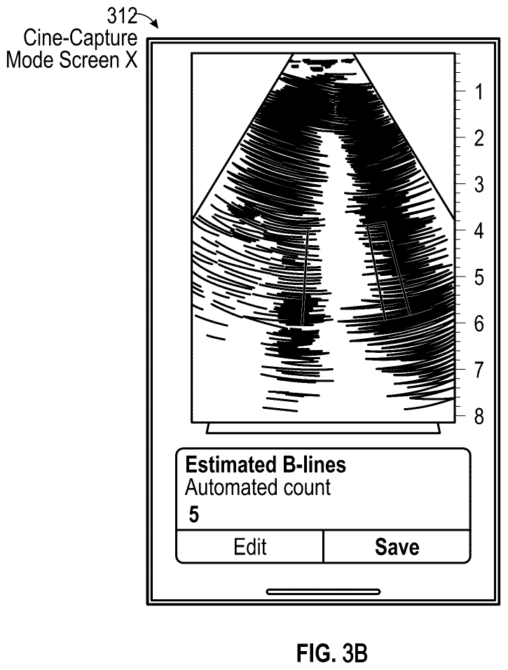

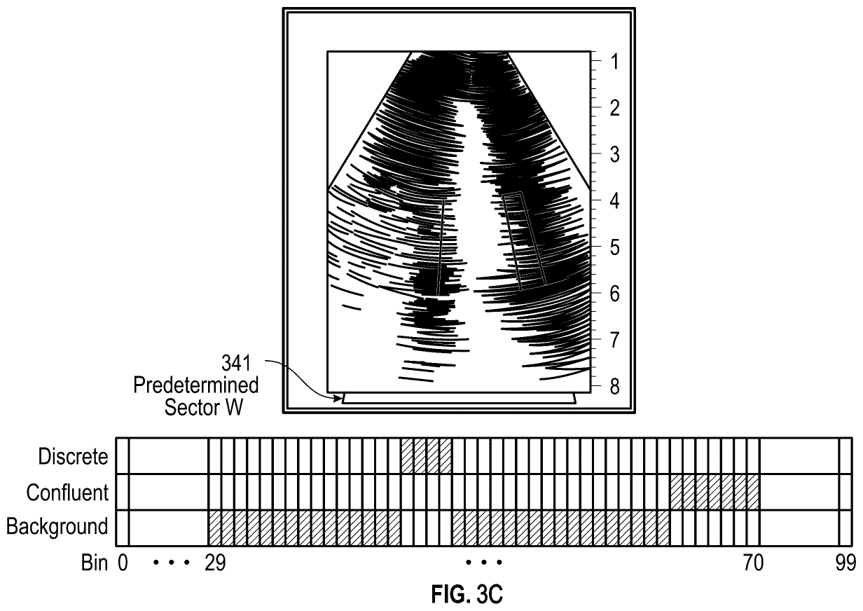

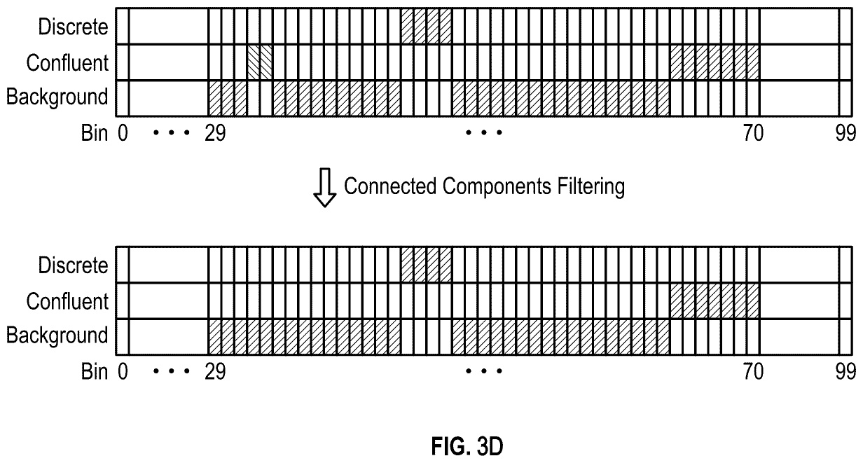

Turning to FIGS. 3A-3B, FIGS. 3A-3B show display screens in accordance with one or more embodiments. In FIG. 3A, a scanning mode screen X 311 is shown for a lung protocol that includes an ultrasound image with a discrete B-line D 321 and a confluent B-line A 331. The ultrasound image in FIG. 3A corresponds to a predetermined sector W 341. The predetermined sector W 341 may be a static 30° sector with a graphical indicator at the bottom of the display screen that shows a user where B-lines may be measured (i.e., the location of various angular bins). The ultrasound image presentation in the scanning mode screen X 311 may also include any potential de-noising or filtering. Likewise, once an automatic B-line counting process is activated, an imaging controller may identify the locations of various B-lines in real-time on the display screen. A scanning mode may be activated once a B-line counter process is selected in a graphical user interface within a selected Lung preset. During a scanning mode, the locations of the B-lines are shown to the user in real-time via overlaid lines shown on the B-Mode image. A B-line segmentation may be a single line for discrete B-lines and a graphical bracket for a B-line segmentation for confluent B-lines. A cine-capture mode screen Y 312 is shown in FIG. 3B after a user touches a GUI button labeled “count” to activate a cine-capture mode and begin recording of a 6 second cine.

In FIG. 3B, a 6 second cine is captured, while B-line segmentations are not presented to the user for each frame. Once the 6 second cine is recorded, the processing device may replay the cine recording to a user and show different types of B-line data. For example, a cine-capture mode screen may provide an overlay of B-line segmentations on each frame, and/or identify maximum number of B-lines observed at a single frame across the recorded cine. As such, display screen may include an output of a B-line count, such as ‘0’, ‘1’, ‘2’, ‘3’, ‘4’, or ‘>5’. Likewise, this count may be manually edited by the user within the graphical user interface. The processing device may also present an error message if a B-line count cannot be performed (e.g., every frame has below minimum image quality). Following an error message, a user may be instructed to reposition the ultrasound device and retry the ultrasound operation.

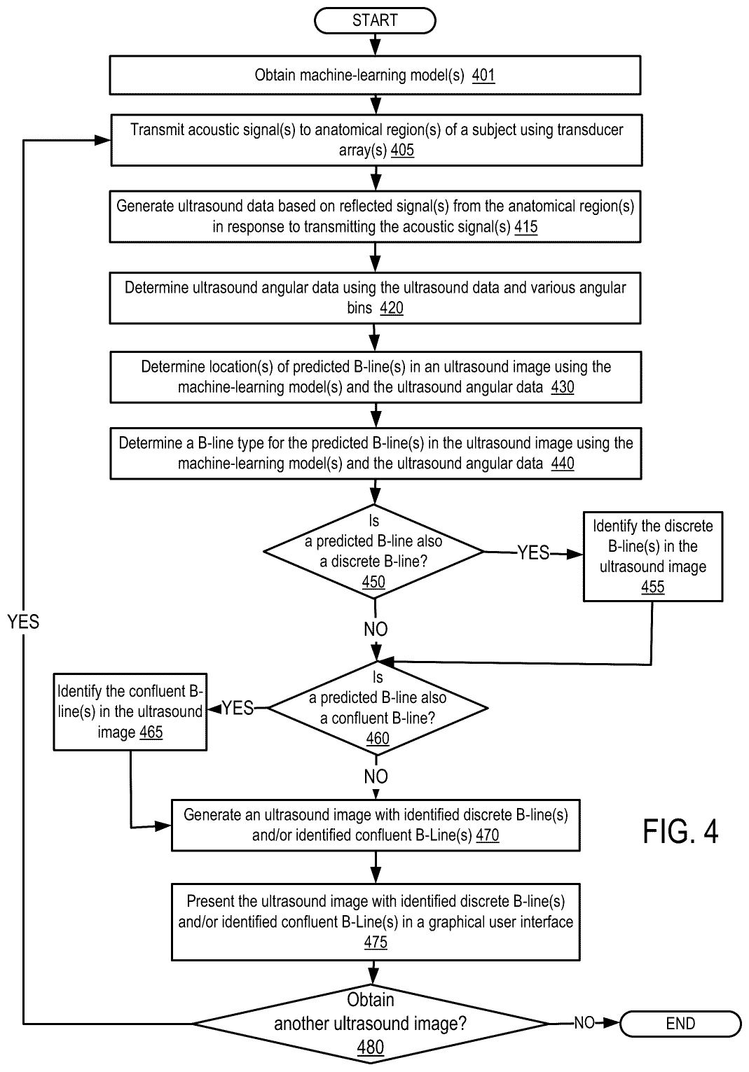

Turning to FIG. 4, FIG. 4 shows a flowchart in accordance with one or more embodiments. Specifically, FIG. 4 describes a general method for predicting B-line data, such as discrete B-lines and/or confluent B-lines, using a machine-learning model. One or more blocks in FIG. 4 may be performed by one or more components (e.g., processing device (704)) as described in FIGS. 1, 2A, 2B, 3A, 3B, and 7-11. While the various blocks in FIG. 4 are presented and described sequentially, one of ordinary skill in the art will appreciate that some or all of the blocks may be executed in different orders, may be combined or omitted, and some or all of the blocks may be executed in parallel. Furthermore, the blocks may be performed actively or passively.

In Block 401, one or more machine-learning models are obtained in accordance with one or more embodiments. In some embodiments, for example, one of the machine-learning models is a deep learning (DL) model with one or more sub-models. For example, sub-models may be similar to other machine-learning models, where the predicted output is used in a post-processing, heuristic method prior to use as an output of the output layer of the overall machine-learning model. In particular, a sub-model may determine a predicted location of one or more B-lines in an ultrasound image. The outputs of this sub-model may then be used in connection with outputs with other sub-models, such as an internal image quality parameter sub-models, for determining a B-line count for a specific cine. Moreover, a machine-learning model may include a global average pooling layer followed by a dense layer and a softmax operation.

In Block 405, one or more acoustic signals are transmitted to one or more anatomical regions of a subject using one or more transducer arrays in accordance with one or more embodiments.

In Block 415, ultrasound data are generated based on one or more reflected signals from one or more anatomical region(s) in response to transmitting one or more acoustic signals in accordance with one or more embodiments.

In Block 420, ultrasound angular data are determined using ultrasound data and various angular bins in accordance with one or more embodiments. In particular, a predetermined sector of an ultrasound beam may be divided into predetermined angular bins for predicting B-lines. Angular bins may identify various angular locations an ultrasound image for detecting B-lines. For example, a middle 30° sector of an ultrasound image may be region of interest undergoing analysis for B-lines. As such, an ultrasound image device divided into 100 bins may only use bins 29-70 (using zero-indexing) as input data for a machine-learning model. This specific range of bins may be indicated in a graphical user interface with a graphical bracket at the bottom of the image. As such, a machine-learning model may return an output only for this selected range of angular bins.

Turning to FIG. 3C, FIG. 3C shows an angular bin layout in accordance with one or more embodiments. In FIG. 3C, a 100-bin layout of for a model that predicts B-line segmentation is shown with one predicted discrete B-line on the left and one confluent B-line on the right. Only the central 30° of the ultrasound image is considered for an inference operation, which corresponds to angular bins 29-70. For the output of a machine-learning model, a respective angular bin may be labeled as part of a discrete B-line, part of a confluent B-line, or background.

Turning to FIG. 3D, FIG. 3D shows connected component filtering in accordance with one or more embodiments. More specifically, if two contiguous angular bins are labeled as confluent by a machine-learning model, the contiguous angular bins would be filtered and considered as background angular bins accordingly. For example, confluent connected components smaller than a predetermined number of bins (e.g., 7 bins) may be converted into background bins through the filtering process.

Returning to FIG. 4, in Block 430, one or more locations of one or more predicted B-line(s) are determined in an ultrasound image using one or more machine-learning models and ultrasound angular data in accordance with one or more embodiments. A machine-learning model may infer that various groups of consecutive angular bins are clustered together with a high probability of the presence B-lines. Thus, different clusters may be determined as including a discrete or a confluent B-line.

In Block 440, a B-line type for one or more predicted B-line(s) is determined in an ultrasound image using one or more machine-learning models and ultrasound angular data in accordance with one or more embodiments. A machine-learning model may determine predicted B-line data for one or more angular bins based on input ultrasound data. For example, different regions of an input image may be classified as being either part of a discrete B-line, a confluent B-line, or other data, such as background data.

In Block 450, a determination is made whether a predicted B-line is also a discrete B-line in accordance with one or more embodiments. Using angular bins and thresholds, for example, a particular number of adjacent bins may identify a discrete B-line, a confluent B-line, and/or background ultrasound data. More specifically, connected components may be processed in a merging and filtering process that smooths and filters angular segmentation data among various bins. For example, a smoothing operation may be used to reduce noise and group adjacent non-background bins. In particular, one or more discrete B-lines may be merged that “touch” confluent B-lines into a larger confluent B-line. Any discrete connected components may be filtered that are smaller than a particular discrete threshold (e.g., 3 bins). Any confluent connected components may be filtered that are smaller than a confluent threshold (e.g., 7 bins). Finally, any discrete connected components may be filtered that are larger than a maximum threshold (e.g., 20 bins) and change the predicted B-line data to identify as confluent B-lines. Some thresholds may be selected based on annotations among clinicians, such as for a training data set. If at least one predicted B-line corresponds to a discrete B-line, the process may proceed to Block 455. If no predicted B-lines correspond to discrete B-lines, the process may proceed to Block 460.

In Block 455, one or more discrete B-lines are identified in an ultrasound image in accordance with one or more embodiments. For example, discrete B-lines may be annotated by overlaying a discrete B-line label on an ultrasound image or cine. Moreover, ultrasound data (such as angular bin data) may be associated with a discrete B-line classification for further processing.

In Block 460, a determination is made whether a predicted B-line is also a confluent B-line in accordance with one or more embodiments. Similar to Block 450, ultrasound data may be predicted to be confluent B-line data. If at least one predicted B-line corresponds to a confluent B-line, the process may proceed to Block 465. If no predicted B-lines correspond to confluent B-lines, the process may proceed to Block 470.

In Block 465, one or more confluent B-lines are identified in an ultrasound image in accordance with one or more embodiments. For example, confluent B-lines may be identified in an ultrasound image in a similar manner as described for discrete B-lines in Block 455.

In Block 470, an ultrasound image is generated with one or more identified discrete B-lines and/or one or more identified confluent B-lines in accordance with one or more embodiments. The ultrasound image may be generated in a similar manner as described above in FIGS. 1 and 7-11 and the accompanying description.

In Block 475, an ultrasound image is presented in a graphical user interface with one or more identified discrete B-lines and/or one or more identified confluent B-lines in accordance with one or more embodiments.

In Block 480, a determination is made whether to obtain another ultrasound image in accordance with one or more embodiments. If another ultrasound image or cine is desired for an anatomical region, the process may proceed to Block 405. If no further ultrasound images are desired by a user, the process may end.

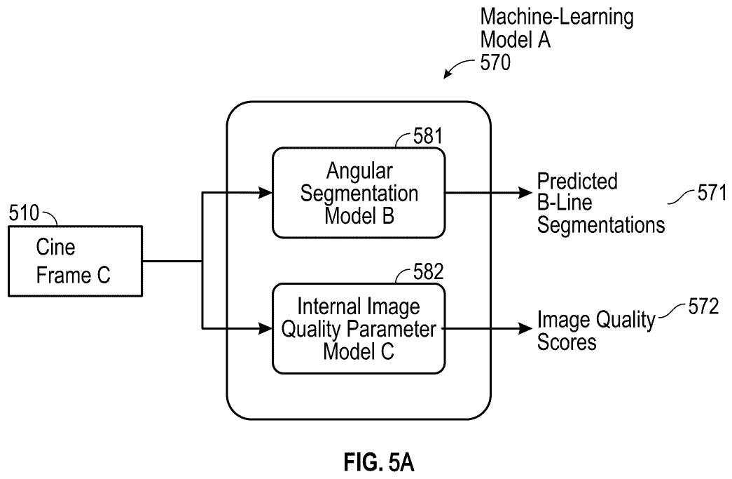

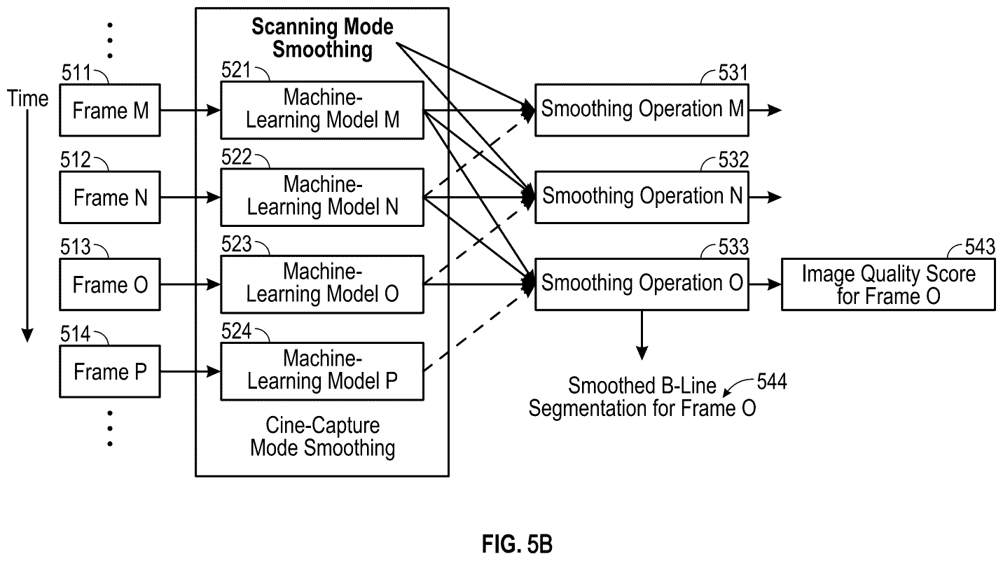

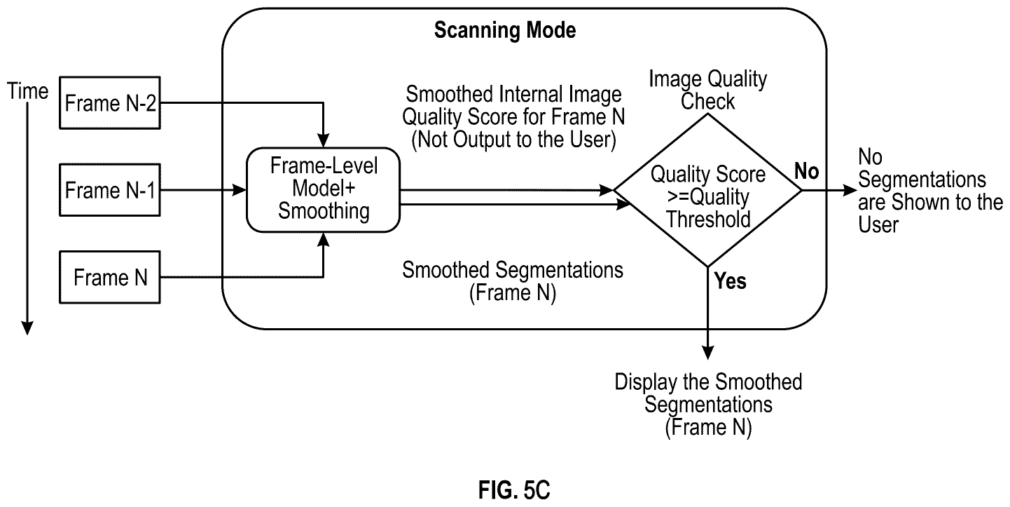

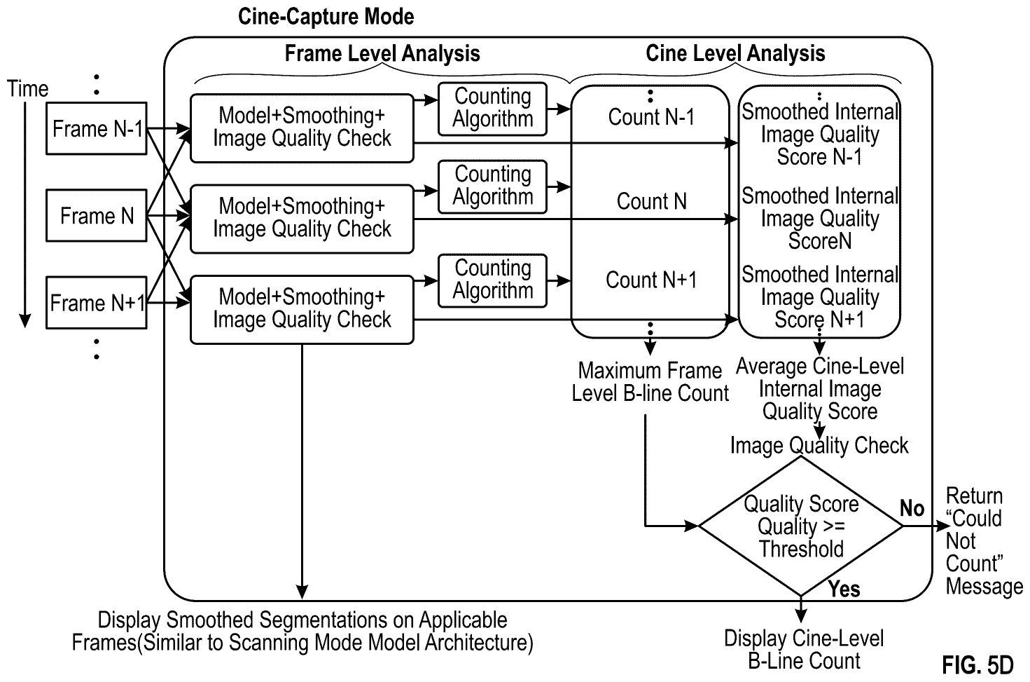

Turning to FIG. 5A, FIG. 5A shows an example of a machine-learning workflow in accordance with one or more embodiments. In FIG. 5A, a cine frame C 510 is input to the machine-learning model A 570, which includes an angular segmentation model B 581 and internal image quality parameter model C 582. The angular segmentation model B 581 determines predicted B-line segmentations 571. For example, the angular segmentation model B 581 may evaluate B-line segmentations performed at the frame level. The results of the segmentations over the span of a cine may be used to produce a B-line count displayed to the user. Additionally, the internal image quality parameter model C 582 determines image quality scores 572. The internal image quality parameter model C 582 may be a classification model that operates on cine frames and produces a value between 0 and 1 for each frame. The machine-learning model A 570 has a frame-level model architecture, where the machine-learning model A 570 may perform an analysis at the frame-level in both a scanning mode and a cine-capture mode. For each frame of a cine, the angular segmentation model B 581 and internal image quality parameter model C 582 are implemented as parallel branches of an artificial neural network. The two models may use the same architecture design. For example, the angular segmentation model B 581 may include an 8-layer convolutional neural network, where each 2-layer block is a convolutional operation followed by a factor of 2 subsampling operation. After the final layer, a global average pooling layer is implemented before providing a predicted output to an output layer.

Turning to FIG. 5B, FIG. 5B shows an example of a machine-learning workflow in accordance with one or more embodiments. In FIG. 5B, a scanning mode smoothing operation is performed for various cine frames, i.e., frame M 511, frame N 512, frame O 513, and frame P 514. These frames are input to respective machine learning models, i.e., machine-learning model M 521, machine-learning model N 522, machine-learning model O 523, and machine-learning model P 524. Predicted B-line segmentation data and quality parameter scores may be temporally smoothed across multiple frames to reduce noise. For example, smoothing operation M 531 may be applied to the output of machine-learning model M 521 as well as machine-learning model N 522 as various previous outputs. Moreover, smoothing operation N 532 may be applied to the output the machine-learning model M 521, machine-learning model N 522, and machine-learning model O 523. Likewise, smoothing operation O 533 may be applied to the output of each machine-learning model shown in FIG. 5B. Thus, an image quality score 543 and a smoothed B-line segmentation 544 may be produced for frame O 513. As shown, the smoothing operation may be performed in a scanning mode using trailing moving average. As such, the predicted output based on the current frame may be averaged together with the predicted outputs for the two preceding frames. In a cine-capture mode, the smoothing process may use symmetric moving average where the current frame is averaged together with the outputs from the prior frame and subsequent frame. Trailing moving average is shown with solid lines in FIG. 5B, while symmetric moving average is shown using segmented lines in FIG. 5B.

Turning to FIG. 5C, FIG. 5C shows a machine-learning workflow of scanning mode in accordance with one or more embodiments. In FIG. 5C, various ultrasound images input to one or more machine-learning models and temporally smoothed using trailing moving averages to produce a resulting smoothed quality score. The resulting smoothed quality score may be compared to a pre-defined image quality threshold set. In some embodiments, for example, a machine-learning model performs better when the input data is of high quality. To facilitate improved image quality, the machine-learning workflow may be used to discard ultrasound images that have a higher likelihood of producing an incorrect B-line count or predicted B-line data. Thus, image quality assessments may include an internal check that is not displayed to the end user. The quality check may be used to facilitate a go/no-go decision about whether to display segmentations and counts to the user. Accordingly, an internal image quality parameter may be used to tune the model performance.

Furthermore, the image quality parameter may include a quality threshold, which may be a fixed value between 0 and 1. A quality score may be a continuous value between 0 and 1 that is determined for various ultrasound images. Furthermore, B-line segmentation predictions may only be displayed to the user if the image quality score is greater than or equal to the image quality threshold. For example, a machine-learning model may review each frame (or cine) and gives it an image quality score between 0 and 1. If the score is greater than or equal to a threshold value, then that frame (or cine) may be deemed to have sufficient quality and predicted B-line data may be displayed to the user. If the quality score is below the threshold, then the system does not display B-line segmentations or B-line counts to the user.

Turning to FIG. 5D, FIG. 5D shows a machine-learning workflow of cine-capture mode in accordance with one or more embodiments. In FIG. 5D, a cine-capture mode performs a frame-level analysis as well as cine-level analysis on the input ultrasound data. The frame-level analysis may produce predicted B-line segmentations that are presented to the user and the cine-level. This frame-level analysis may also produce the B-line count displayed to the user. Similar to a scanning mode, B-line segmentation predictions for a frame may be displayed to the user if the image quality score is greater than or equal to the image quality in the cine-capture mode. In addition, for each captured frame, the B-line angular segmentations are passed to a counting algorithm that determines per-frame B-line counts, such as using an instant-percent method. Only frames with image quality scores greater than or equal to the threshold may be used for the overall B-line count prediction. At the cine-level, after each frame is processed, a counting algorithm may analyze each frame in an entire cine to determine the maximum B-line count from any single-frame (e.g., multiple frames within the cine may have the maximum B-line count). This maximum frame count may be logged as the B-line count for the cine. The average image quality score may also be determined across the entire cine. If the cine's average image quality score is above the predefined image quality threshold, then the determined B-line count may be presented to the user. Otherwise, no B-line count may be returned to the user and error message may be displayed. Thus, B-line count predictions may be filtered out using one or more image quality checks at the cine-level to improve model confidence and accuracy.

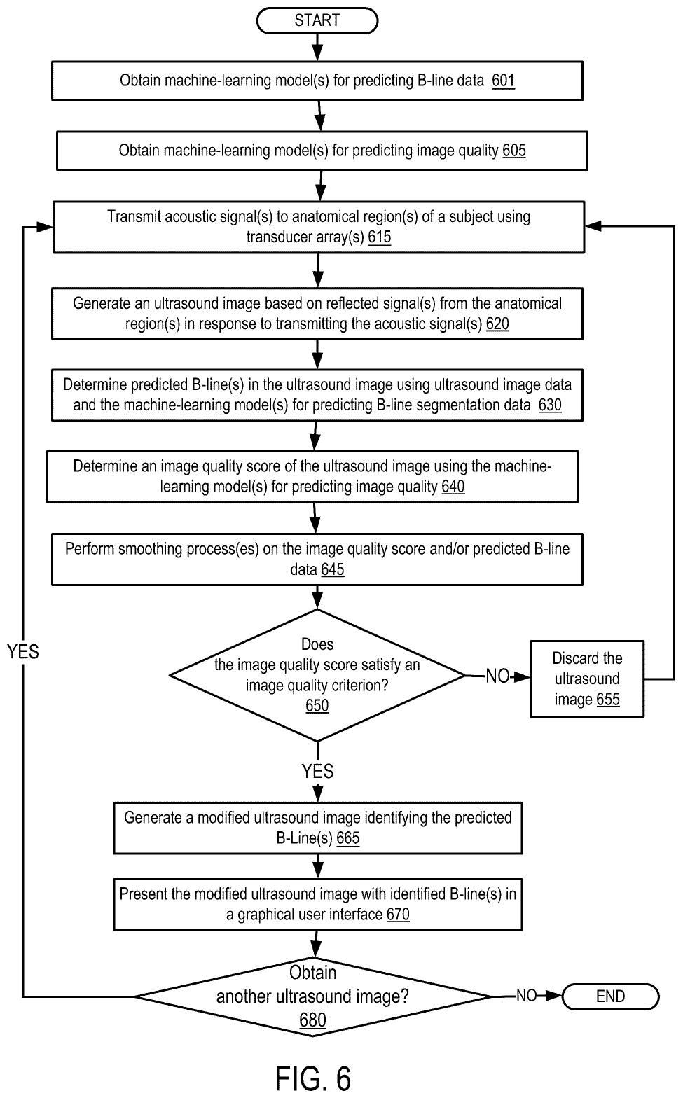

Turning to FIG. 6, FIG. 6 shows a flowchart in accordance with one or more embodiments. Specifically, FIG. 6 describes a general method for predicting B-line data using machine learning and quality control. One or more blocks in FIG. 6 may be performed by one or more components (e.g., processing device (704)) as described in FIGS. 1, 2A, 2B, 3A, 3B, 5A, 5B, 5C, 5D, and 7-11. While the various blocks in FIG. 6 are presented and described sequentially, one of ordinary skill in the art will appreciate that some or all of the blocks may be executed in different orders, may be combined or omitted, and some or all of the blocks may be executed in parallel. Furthermore, the blocks may be performed actively or passively.

In Block 601, one or more machine-learning models are obtained for predicting B-line data in accordance with one or more embodiments.

In Block 605, one or more machine-learning models are obtained for predicting image quality in accordance with one or more embodiments.

In Block 615, one or more acoustic signals are transmitted to one or more anatomical regions of a subject using one or more transducer arrays in accordance with one or more embodiments.

In Block 620, an ultrasound image is generated based on one or more reflected signals from anatomical regions in response to transmitting one or more acoustic signals in accordance with one or more embodiments.

In Block 630, one or more predicted B-lines are determined in an ultrasound image using ultrasound image data and one or more machine-learning models in accordance with one or more embodiments.

In Block 640, an image quality score of an ultrasound image is determined using one or more machine-learning models in accordance with one or more embodiments. For example, the image quality score may determine an accuracy of predicted results from a machine-learning model. In particular, image quality scores may be used to determine whether an ultrasound image or frames in a cine) are of sufficient quality to display B-lines counts and B-line angular segmentations to the user.

In Block 645, one or more smoothing processes are performed on an image quality score and/or predicted B-line data in accordance with one or more embodiments.

In Block 650, a determination is made whether an image quality score satisfies an image quality criterion in accordance with one or more embodiments. The image quality criterion may include one or more quality thresholds for determining whether an ultrasound image or cine has sufficient quality for detecting B-lines. For quality thresholds, quality threshold may be determined based on correlation coefficients between a machine-learning model's predicted B-line count and that of a “ground truth” estimate, which may be a median annotator count of B-lines. Because the choice of a quality threshold under a cine-capture mode may affect the performance of a machine-learning, an intraclass correlation (ICC) may be determined as a function of a specific quality threshold or quality operating point. Likewise, the lowest image quality threshold may be selected that is permissible as input data while also maintaining the required level of B-lines counting agreement with acquired data from clinicians. Likewise, other image quality criteria are contemplated based on analyzing ultrasound images, patient data, and other input features. If a determination is made that an image quality score fails to satisfy the image quality criterion, the process may proceed to Block 655. If a determination is made that the image quality score satisfies the image quality criterion, the process may proceed to Block 665.

In Block 655, an ultrasound image is discarded in accordance with one or more embodiments. An ultrasound image or frame may be ignored for use in a machine-learning workflow. Likewise, the ultrasound image or frame may be deleted from memory in a processing device accordingly.

In Block 665, a modified ultrasound image is generated that identifies one or more predicted B-lines in accordance with one or more embodiments. For example, the modified ultrasound image may be the original image obtained from an ultrasound device with one or more B-line overlays on the original image along with other superimposed information, such as B-line count data.

In Block 670, a modified ultrasound image is presented in a graphical user interface with one or more identified B-lines in accordance with one or more embodiments.

In Block 680, a determination is made whether to obtain another ultrasound image in accordance with one or more embodiments. If another ultrasound image or cine is desired for an anatomical region, the process may proceed to Block 615. If no further ultrasound images are desired by a user, the process may end.

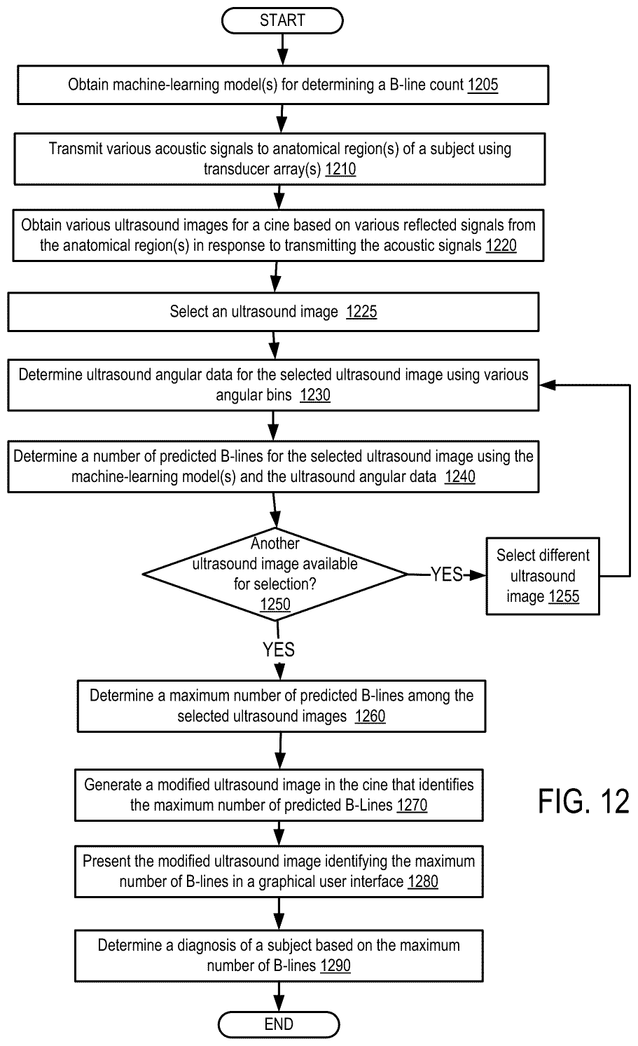

Turning to FIG. 12, FIG. 12 shows a flowchart in accordance with one or more embodiments. Specifically, FIG. 12 describes a general method for counting a number of B-lines in an ultrasound image or cine using machine learning. One or more blocks in FIG. 12 may be performed by one or more components (e.g., processing device (704)) as described in FIGS. 1, 2A, 2B, 3A, 3B, 5A, 5B, 5C, 5D, and 7-11. While the various blocks in FIG. 12 are presented and described sequentially, one of ordinary skill in the art will appreciate that some or all of the blocks may be executed in different orders, may be combined or omitted, and some or all of the blocks may be executed in parallel. Furthermore, the blocks may be performed actively or passively.

In Block 1205, one or more machine-learning models are obtained for determining a B-line count in accordance with one or more embodiments. For example, a B-line count may be determined using a rule-based process that obtains predicted B-line data from one or more machine-learning models. In particular, a number of distinct B-line segmentations may be converted into a particular B-lines count (e.g., a total number of discrete and/or confluent B-lines in a cine). Using a connected components approach, contiguous bins with predictions of a certain class (e.g., discrete or confluent) may be determined to be candidate B-lines. Within a counting algorithm, the B-line segmentation predictions are used to determine a B-lines count prediction from each frame. Thus, a counting algorithm may analyze multiple frames in a cine to determine the maximum count of B-lines among the analyzed frames in a cine loop. This maximum frame count may be presented to a user in a graphical user interface as the B-line count for the cine. In some embodiments, the B-line count may only be presented to the user if the majority of the frames in the cine are determined to be measurable. Otherwise, a user may receiver a message indicating that the predicted B-line counts cannot be determined.

In Block 1210, various acoustic signals are transmitted to one or more anatomical regions of a subject using one or more transducer arrays in accordance with one or more embodiments.

In Block 1220, various ultrasound images are obtained for a cine based on various reflected signals from one or anatomical regions in response to transmitting various acoustic signals in accordance with one or more embodiments.

In Block 1225, an ultrasound image is selected in accordance with one or more embodiments. For example, one frame within a recorded cine may be selected for a B-line analysis.

In Block 1230, ultrasound angular data are determined for a selected ultrasound image using various angular bins in accordance with one or more embodiments.

In Block 1240, a number of predicted B-lines are determined for a selected ultrasound image using one or more machine-learning models and ultrasound angular data in accordance with one or more embodiments. Likewise, the selected ultrasound image may be ignored if the image fails to satisfy an image quality criterion.

In Block 1250, a determination is made whether another ultrasound image is available for selection in accordance with one or more embodiments. For example, frames in a cine may be iteratively selected until every frame is analyzed for predicted B-lines. If another image is available (e.g., not all frames have been selected in a cine), the process may proceed to Block 1255. If no more images are available for selection, the process may proceed to Block 1260.

In Block 1255, a different ultrasound image is selected in accordance with one or more embodiments.

In Block 1260, a maximum number of predicted B-lines are determined among various selected ultrasound images in accordance with one or more embodiments. Based on analyzing the selected images, a maximum number of predicted B-lines may be determined accordingly.

In Block 1270, a modified ultrasound image in a cine is generated that identifies a maximum number of predicted B-lines in accordance with one or more embodiments.

In Block 1280, a modified ultrasound image is presented in a graphical user interface that identifies the maximum number of B-lines in accordance with one or more embodiments.

In Block 1290, a diagnosis of a subject is determined based on a maximum number of B-lines in accordance with one or more embodiments.

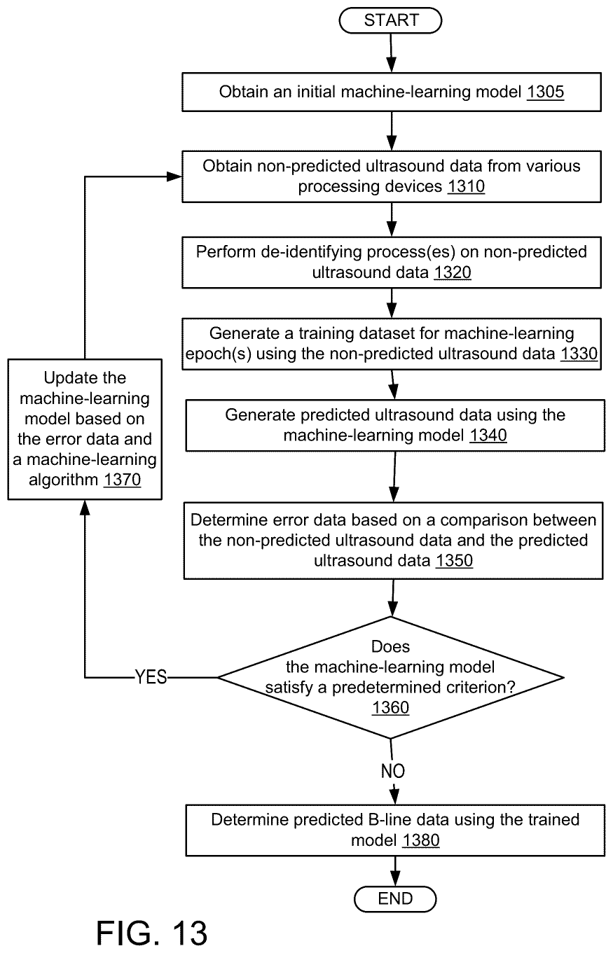

Turning to FIG. 13, FIG. 13 shows a flowchart in accordance with one or more embodiments. Specifically, FIG. 13 describes a general method for training a machine-learning model to predict ultrasound data. One or more blocks in FIG. 13 may be performed by one or more components (e.g., processing device (704)) as described in FIGS. 1, 2A, 2B, 3A, 3B, 5A, 5B, 5C, 5D, and 7-11. While the various blocks in FIG. 13 are presented and described sequentially, one of ordinary skill in the art will appreciate that some or all of the blocks may be executed in different orders, may be combined or omitted, and some or all of the blocks may be executed in parallel. Furthermore, the blocks may be performed actively or passively.

In Block 1305, an initial machine-learning model is obtained in accordance with one or more embodiments. The machine-learning model may be similar to the machine-learning models described above.

In Block 1310, non-predicted ultrasound data are obtained from various processing devices in accordance with one or more embodiments. In some embodiments, non-predicted ultrasound data are acquired using a cloud-based approach. For example, a cloud server may be a remote server (i.e., remote from a site of an ultrasound operation that collected original ultrasound data from living subjects) that acquires ultrasound data from patients at multiple clinical sites geographical separated. The collected images for the non-predicted ultrasound data may represent the actual user base of clinicians and their patients. In other words, the non-predicted ultrasound data may be obtained as part of real clinical scans. Because non-predicted data is being sampled from examinations performed in the field, the cloud server may not have access to information such as gender and age associated with the collected ultrasound data. Likewise, clinicians may upload ultrasound scans and patient metadata over a network for use in a training dataset.

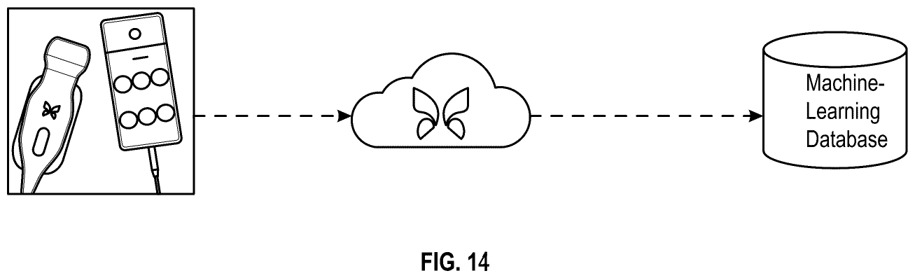

Furthermore, some patient studies may be exported to a cloud server in addition to samples of individual images. For example, if multiple patient studies are transmitted to a machine-learning database on a particular day, some patient studies may be used for development purposes and for evaluations. Likewise, various filters may be applied to ultrasound data obtained at a cloud server to select for training operations. In some embodiments, a machine-learning model for predicting B-line data may only use ultrasound images acquired with a Lung preset. Likewise, another filter may only include ultrasound data for recorded cines of 8 cm or greater depth. A particular depth filter may be used, such as due to the reliability of shallow images for evaluating lungs for B-lines. Likewise, ultrasound images with pleural effusion may be excluded from a training dataset, because they may be inappropriate for B-lines. In particular, parameters that influence the detection, number, size, and shape of B-lines may result in the presence of a pleural effusion. FIG. 14 shows an example of a data ingestion process for collecting non-predicted data for a machine-learning database.

In Block 1320, one or more de-identifying process on non-predicted ultrasound data in accordance with one or more embodiments. Once ultrasound data is uploaded to one or more cloud servers, ultrasound data may be processed before being transmitted to a machine-learning database for use in the development of machine-learning tools. For example, a machine-learning model may be trained using ultrasound scans along with limited, anonymized information about the source and patient demographics. After an ultrasound image and patient data are uploaded to a cloud server, a de-identifying process may be performed to anonymize the data before the uploaded data is accessible for machine learning. A de-identifying process may remove personal health information (PHI) and personal identifiable information (PII) from image, such as according to a HIPAA safe harbor method. Once this anonymizing is performed, the image data may be copied to a machine-learning database for use in constructing datasets for training and evaluation.

In some embodiments, an anonymized patient identifier is not available for developing and evaluating a machine-learning model. Consequently, a study identifier may be used as a proxy for a patient identifier. As such, a study identifier may indicate that a set of images that were acquired during one examination on a particular day. The consequence of not having any PII is that if a patient had, for example, two exams a day apart, an image from the first study could be in one dataset and an image from the second study could be in another dataset. By using probe positioning, ultrasound images that result in the same scan of the same patient would not be similar. Likewise, geographical diversity of training data may result in the same patient not being in the same dataset multiple times.

In Block 1330, a training dataset is generating for one or more machine-learning epochs using non-predicted ultrasound data in accordance with one or more embodiments. The training data may be used in one or more training operations to train and evaluate one or more machine-learning models. For example, the volume of data made available to a cloud server for training may be orders of magnitude larger than the amounts of data typically used for clinical studies. Using this volume of data, natural variations of ultrasound exams may be approximated for actual performance in clinical settings. Training data may include data for actual training, validation, and/or final testing of a trained model. Additionally, training data may be sampled randomly from cloud data over a diverse geographical population. Likewise, training data may include annotations from human experts that are collected based on specific instructions for performing the annotation. For example, an ultrasound image may be annotated to identify the number of B-lines in the image as well as tracing a width of observed B-lines for use in segmenting the B-lines in each frame.

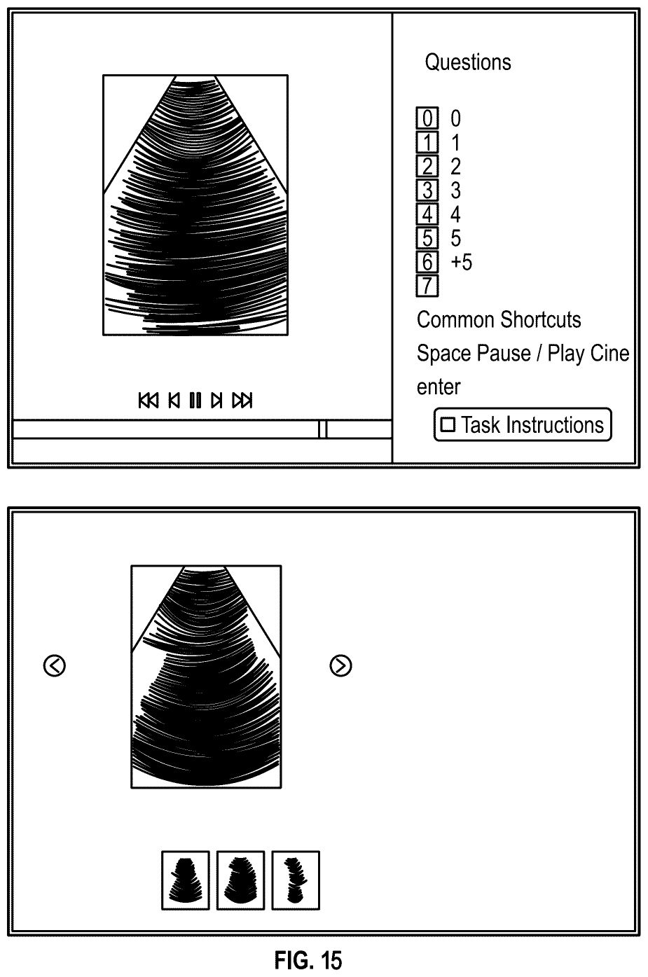

Turning to FIG. 15, FIG. 15 shows a user interface tool for labeling non-predicted ultrasound images to produce training data with annotations. In FIG. 15, the upper image includes an annotation tool interface as presented to clinicians and other users for the lung-b-line-count task. The lower image of FIG. 15 shows a set of sample interpretations with descriptions for the task to be performed. The section on the right of FIG. 15 shows the user instructions for this task.

In some embodiments, an initial model is trained using ultrasound images produced as part of the lung-measurability task. For example, individual frames of a lung cine may be annotated as either measurable or not measurable for assessing the presence of B-lines. For each frame, a model may be trained by being presented with the frame image and each annotator's separate binary label (e.g., background or B-line) for that image. Some training operations may be implemented as a logistic regression problem, with its ideal output being analogous to a fraction of annotators who determine a B-line for the image presented. A supervised learning algorithm may be subsequently used as the machine-learning algorithm.

In some embodiments, for example, a training dataset for predicting B-lines is based on lung-b-line-count data annotations. To perform a random sampling for B-line training data, a query for lung ultrasound cines may be performed against one or more machine-learning databases. For example, the instructions for an annotator may include the following: “You are presented with a lung cine. Please annotate whether the cine contains a pleural effusion and it is therefore inappropriate to use it to count B-lines.” For cines, cines that include B-lines may also be identified by annotators via the lung-b-line-presence task. During this task, annotators may classify cines according to one or more labels: (1) having B-lines, (2) maybe having B-lines, (3) being appropriate images for assessing B-lines but not containing B-lines, or (4) being inappropriate for assessing the presence of B-lines.

For illustrative purposes, an annotator may be presented with a short 11-frame cine for identifying lung-b-line-segmentation. In this task, a middle frame is the frame of interest to be labeled. The annotator may label the middle frame using a drawing tool to trace the width of the observed B-lines and indicate whether they believed those B-lines to be discrete or confluent. The middle frame of the cine may be annotated to ensure parity among the annotators and establish agreement or disagreement on the presence of B-line(s) in that frame. For context, the annotators may also be provided with the frames before and the frames after the middle frame.

In Block 1340, predicted ultrasound data are generated using a machine-learning model in accordance with one or more embodiments.

In Block 1350, error data are determined based on a comparison between non-predicted ultrasound data and predicted ultrasound data in accordance with one or more embodiments. For example, error data may be determined using a loss function with various components. In some embodiments, for example, the discrete, confluent, and background labels are used to calculate cross-entropy loss for an image, e.g., in a similar manner as used to train various segmentation deep learning models such as U-nets. Another component is a counting-error loss for an image. By applying the connected components filtering and counting method, to both the model's B-line segmentation output and an annotator's segmentation labels, error for predicted B-line segmentation data may be determined based on the image's overall B-lines predicted count.

In Block 1360, a determination is made whether a machine-learning model satisfies a predetermined criterion in accordance with one or more embodiments. If the machine-learning model satisfies the predetermined criterion (e.g., a predetermined degree of accuracy or training over a specific number of iterations), the process may proceed to Block 1380. If the machine-learning model fails to satisfy the predetermined criterion, the process may proceed to Block 1370.

In Block 1370, a machine-learning model is updated based on error data and a machine-learning algorithm in accordance with one or more embodiments. For example, the machine-learning model may be a backpropagation method that updates the machine-learning model using gradients. Likewise, other machine-learning models are contemplated, such as ones using synthetic gradients. After obtaining an updated model, the updated model may be used to determined predicted data again with the previous workflow.

In Block 1380, predicted B-line data are determined using a trained model in accordance with one or more embodiments.

### Non-GUI Interaction Features and Simplified Workflows Using Artificial Intelligence

Some embodiments provide systems and methods for managing ultrasound exams. Ultrasound exams may include use of an ultrasound imaging device in operative communication with a processing device, such as a phone, tablet, or laptop. The phone, tablet, or laptop may allow for control of the ultrasound imaging device and for viewing and analyzing ultrasound images. Some embodiments include reducing graphical user interface (GUI) interactions with such a processing device using voice commands, automation, and/or artificial intelligence. For example, various non-GUI inputs and non-GUI outputs may provide one or more substitutes for typical GUI interactions, such as the following: (1) starting up the ultrasound app; (2) logging into a user account or organization's account; (3) selecting an exam type; (4) selecting an ultrasound mode (e.g., B-mode, M-mode, Color Doppler mode, etc.); (5) selecting a specific preset and/or other set of parameters (e.g., gain, depth, time gain compensation (TGC)); (6) being guided to the correct probe location for imaging a desired anatomical region of interest; (7) capturing an image or cine; (8) inputting patient info; (9) completing worksheets; (10) signing the ultrasound study; and (11) uploading the ultrasound study. Non-GUI inputs may also include inputs from artificial intelligence functions and techniques, where an input is automatically selected without a user interacting with an input device or user interface.

Some embodiments provide systems and methods for simplifying workflows during ultrasound examinations. For example, a particular ultrasound imaging protocol may include the capturing of ultrasound images or cines from multiple anatomical regions. A simplified workflow may involve some or all of the following features:
1. Automatically selecting imaging mode, preset, gain, depth, and/or TGC optimized for collecting clinically relevant images for the anatomy scanned in one scan in the protocol;
2. Presenting a probe placement guide prior to and/or during ultrasound scanning for correctly placing the ultrasound imaging device in order to capture clinically relevant images for the scan in the protocol;
3. Presenting guidance, such as a quality indicator and/or anatomical labels, during ultrasound scanning for correctly placing the ultrasound imaging device;
4. Automatically capturing ultrasound images for the scan when the quality of the collected images exceeds a threshold;
5. Automatically proceeding to repeat the above process for the next scan in the protocol; and
6. When the protocol is complete, provide a summary of the exam and provide an option for the user to review the captured images.

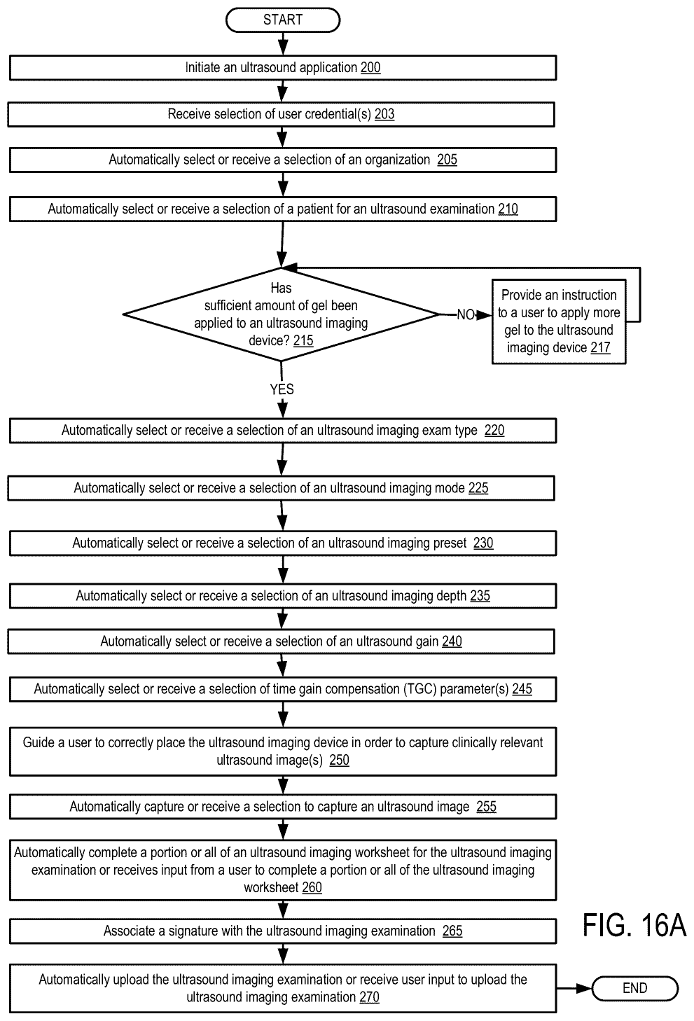

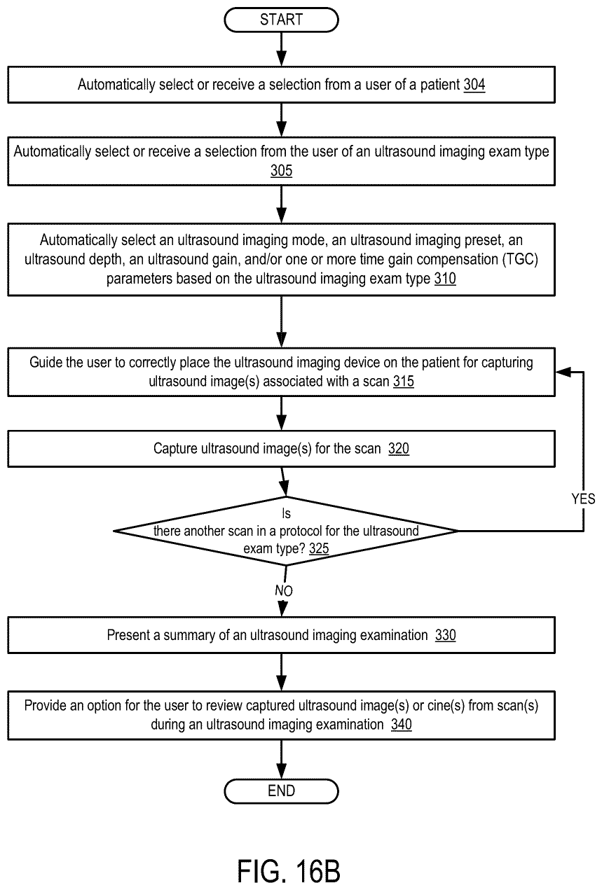

Turning to FIG. 16A, FIG. 16A shows a flowchart in accordance with one or more embodiments. Specifically, FIG. 16A describes a method for performing one or more ultrasound scans using non-GUI inputs to a processing device. The blocks in FIG. 16A may be performed by a processing device (e.g., the processing device 104) in communication with an ultrasound device (e.g., the ultrasound device 102). While the various blocks in FIG. 16A are presented and described sequentially, one of ordinary skill in the art will appreciate that some or all of the blocks may be executed in different orders, may be combined or omitted, and some or all of the blocks may be executed in parallel. Furthermore, the blocks may be performed actively or passively.

In Block 200, the processing device initiates an ultrasound application in accordance with one or more embodiments. In some embodiments, an ultrasound application may automatically start up when the processing device is connected to or plugged into an ultrasound imaging device, such as using an automatic wireless connection or wired connection. In some embodiments, an ultrasound application may be initiated using voice control, such as by a user providing a voice command. For example, the user may state “start scanning” and/or the processing device may state over a voice message “would you like to start scanning,” and a user may respond to the voice message with a voice command that includes “start scanning.” It should be appreciated that for any phrases described herein as spoken by the user or the processing (e.g., “start scanning”), the exact phrase is not limiting, and other language that conveys a similar meaning may be used instead.

In some embodiments, an ultrasound application is automatically initiated in response to triggering an input device on an ultrasound imaging device. For example, the ultrasound application may start after a user presses a button on an ultrasound probe. In some embodiments, the processing device detecting an ultrasound imaging device within a predetermined proximity may also automatically initiate the ultrasound application.

In Block 203, the processing device receives a selection of one or more user credentials in accordance with one or more embodiments. For example, the processing device may receive a voice-inputted password, perform facial recognition of a user, perform fingerprint recognition of the user, or perform voice recognition of a user in order to allow the user to continue to access the ultrasound application.

In Block 205, the processing device automatically selects or receives a selection of an organization in accordance with one or more embodiments. The organization may be, for example, a specific healthcare provider (e.g., a hospital, clinic, doctor's office, etc.) In some embodiments, the selected organization may correspond to a default organization for a particular user of the ultrasound application. In some embodiments, the selected organization may correspond to a predetermined default organization associated with the specific ultrasound imaging device. In such embodiments, the processing device may access a database that associates various organizations with probe serial numbers and/or other device information. In some embodiments, a user selects an organization using voice commands or other voice control. For example, the processing device may output using an audio device a request for an organization and the user may respond with identification information for the desired organization (e.g., a user may audibly request for the ultrasound application to use “St. Elizabeth's organization”). In some embodiments, the processing device automatically selects an organization based on location data, such as global positioning system (GPS) coordinates acquired from a processing device. For example, if a doctor is located at St. Elizabeth's medical center, the ultrasound application may automatically use St. Elizabeth's medical center as the organization.

In Block 210, the processing device automatically selects or receives a selection of a patient for the ultrasound examination in accordance with one or more embodiments. In some embodiments, the processing device may automatically identify the patient using machine-readable scanning of a label associated with the patient. The label scanning may include, for example, barcode scanning, quick response (QR) code scanning, or radio frequency identification (RFID) scanning. In some embodiments, a processing device performs facial recognition of a patient to determine which patient is being examined. However, other types of automated recognition processes are also contemplated, such as fingerprint recognition of a patient or voice recognition of the patient. In some embodiments, patient data is extracted from a medical chart or other medical documents. In such embodiments, a doctor may show the chart to a processing device's camera. In some embodiments, the processing device may automatically obtain the patient's data may from a personal calendar. For example, the processing device may access a current event on a doctor's calendar (stored on the processing device or accessed by the processing device from a server) that says “ultrasound for John Smith DOB 1/8/42.” In some embodiments, a user may select a patient using a voice command. In such embodiments, a user may identify a patient being given the examination (e.g., the user announces, “John Smith birthday 1/8/42,” and/or the processing device says “What is the patient's name and date of birth?” and the user responds). In some embodiments, a processing device may request patient information at a later time by email or text message.

Applying sufficient ultrasound coupling medium (referred to herein as “gel”) to the ultrasound device may be necessary to collect clinically usable ultrasound images. In Block 215, the processing device automatically determines whether a sufficient amount of gel has been applied to an ultrasound imaging device in accordance with one or more embodiments. The processing device may automatically detect whether sufficient gel is disposed on an ultrasound imaging device based on one or more collected ultrasound images (e.g., the most recently collected ultrasound image, or a certain number of the most recently collected ultrasound images). In some embodiments, the processing device may use a statistical model to determine whether sufficient gel is disposed on an ultrasound device. The statistical model may be stored on the processing device, or may be stored on another device (e.g., a server) and the processing device may access the statistical model on that other device. The statistical model may be trained on ultrasound images labeled with whether they were captured when the ultrasound imaging device had sufficient or insufficient gel on it. Further description may be found in U.S. patent application Ser. No. 17/841,525, the content of which is incorporated by reference herein in its entirety.

Based on determining in Block 215 that a sufficient amount of gel has not been applied to the ultrasound device, the processing device proceeds to Block 217. In Block 217, the processing device provides an instruction to the user to apply more gel to the ultrasound imaging device in accordance with one or more embodiments. For example, the processing device may provide voice guidance to a user, e.g., the processing device may say “put more gel on the probe.” The processing device then returns to Block 215 to determine whether sufficient gel is now on the ultrasound imaging device.

Based on determining in Block 215 that a sufficient amount of gel has been applied to the ultrasound device, the processing device proceeds to Block 220. In Block 220, the processing device automatically selects or receives a selection of an ultrasound imaging exam type in accordance with one or more embodiments. In some embodiments, a user may select a particular exam type using voice control or voice commands (e.g., user says “eFast exam” and/or the processing device says “What is the exam type?” and the user responds with a particular exam type). In some embodiments, a processing device may automatically pull an exam type from a calendar. For example, the current event on a doctor's calendar (stored on the processing device or accessed by the processing device from a server) may identify an eFAST exam for John Smith DOB 1/8/42.

In Block 225, the processing device automatically selects or receives a selection of an ultrasound imaging mode in accordance with one or more embodiments. In some embodiments, a processing device may automatically determine a mode for a particular exam type (selected in Block 220). For example, if the exam type is an ultrasound imaging protocol that includes capturing B-mode images, the processing device may select B-mode. In some embodiments, the processing device may automatically select a default mode (e.g., B-mode). In some embodiments, a user may select a particular mode using voice control. For example, a user may provide a voice command identifying “B-mode” and/or the processing device may use a voice message to request which mode is selected by a user (such as the processing device stating “what mode would you like” and the user responding).

In Block 230, the processing device automatically selects or receives a selection of an ultrasound imaging preset in accordance with one or more embodiments. In some embodiments, the processing device may automatically select the preset based on the exam type. For example, if the exam type is an ultrasound imaging protocol that includes capturing images of the lungs, the processing device may select a lung preset. In some embodiments, a user may select a preset using voice control or a voice command (e.g., a processing device may request a user to identify which preset to use for an examination and/or the user may simply say “cardiac preset”). In some embodiments, a default preset may be selected for a particular user of an ultrasound imaging device, a particular patient, or a particular organization.

In some embodiments, a processing device retrieves an electronic medical record (EMR) of a subject and selects the ultrasound imaging preset based on the EMR. For example, after pulling data from a patient's record, a processing device may automatically determine that the patient has breathing problems and select a lung preset accordingly. In some embodiments, the processing device may retrieve a calendar of the user and select the ultrasound imaging preset based on the calendar. For example, the processing device may pull data from a doctor's calendar (e.g., stored on the processing device or accessed by the processing device from a server) to determine which preset to use for a patient (e.g., the current event on the doctor's calendar says lung ultrasound for John Smith DOB 1/8/42 and the processing device automatically selects a lung preset).

In some embodiments, a processing device automatically determines an anatomical feature being imaged and automatically selects, based on the anatomical feature being imaged, an ultrasound imaging preset corresponding to the anatomical feature. In some embodiments, artificial intelligence (AI)-assisted imaging is used to determine anatomical locations being imaged (e.g., using statistical models and/or deep learning techniques) and the identified anatomical location may be used to automatically select an ultrasound imaging preset corresponding to the anatomical location. Further description of automatic selection of presets may be found in U.S. patent application Ser. Nos. 16/192,620, 16/379,498, and 17/031,786, the contents of which are incorporated by reference herein in their entireties.

In Block 235, the processing device automatically selects or receives a selection of an ultrasound imaging depth in accordance with one or more embodiments. In some embodiments, a processing device automatically sets the ultrasound imaging depth for a particular scan, such as based on a particular preset or a statistical model trained to determine an optimal depth for an inputted image. In some embodiments, a user may use voice control or a voice command to adjust the imaging depth (e.g., a user may say “increase depth” and/or the processing device may request using audio output whether to adjust the depth and the user may respond).

In Block 240, the processing device automatically selects or receives a selection of an ultrasound gain in accordance with one or more embodiments. In some embodiments, a processing device automatically sets the gain for a particular scan, such as based on a particular preset or a statistical model trained to determine an optimal gain for an inputted image. In some embodiments, a user may use voice control or voice commands to adjust the gain (e.g., a user may say “increase gain” and/or the processing device may request using audio output whether to adjust the gain and the user responds).

In Block 245, the processing device automatically selects or receives a selection of one or more time gain compensation (TGC) parameters in accordance with one or more embodiments. In some embodiments, for example, a user uses voice control and/or voice commands to adjust the TGC parameters for an ultrasound scan. In some embodiments, a processing device automatically sets the TGC such as based on a particular preset or using a statistical model trained to determine an optimal TGC for a given inputted image.

In Block 250, the processing device guides a user to correctly place the ultrasound imaging device in order to capture one or more clinically relevant ultrasound images in accordance with one or more embodiments. In some embodiments, a processing device may provide a series of instructions or steps using a display device and/or an audio device to assist a user in obtaining a desired ultrasound image. For example, the processing device may use images, videos, audio, and/or text to instruct the user where to initially place the ultrasound imaging device. As another example, the processing device may use images, videos, audio, and/or text to instruct the user to translate, rotate, and/or tilt the ultrasound imaging device. Such instructions may include, for example, “TURN CLOCKWISE,” “TURN COUNTER-CLOCKWISE,” “MOVE UP,” “MOVE DOWN,” “MOVE LEFT,” and “MOVE RIGHT.”

In some embodiments, a processing device provides a description of a path that does not explicitly mention the target location, but which includes the target location, as well as other non-target locations. For example, non-target locations may include locations where ultrasound data is collected that is not capable of being transformed into an ultrasound image of the target anatomical view. Such a path of target and non-target locations may be predetermined in that the path may be generated based on the target ultrasound data to be collected prior to the operator beginning to collect ultrasound data. Moving the ultrasound device along the predetermined path should, if done correctly, result in collection of the target ultrasound data. The predetermined path may include a sweep over an area (e.g. a serpentine or spiral path, etc.). The processing device may output audio instructions for moving the ultrasound imaging device along the predetermined path. For example, the instruction may be “move the ultrasound probe in a spiral path over the patient's torso.” The processing device may additionally or alternatively output graphical instructions for moving the ultrasound imaging device along the predetermined path.

In some embodiments, the processing device may provide an interface whereby a user is guided by one or more remote experts that provide instructions in real-time based on viewing the user or collected ultrasound images. Remote experts may provide voice instructions and/or graphical instructions that are output by the processing device.

In some embodiments, the processing device may determine a quality of ultrasound images collected by the ultrasound imaging device and output the quality. For example, the outputted quality may be through audio (e.g., “the ultrasound images are low quality” or “the ultrasound images have a quality score of 25%”) and/or through a graphical quality indicator.

In some embodiments, the processing device may determine anatomical features present and/or absent in ultrasound images collected by the ultrasound imaging device and output information about the anatomical features. For example, the outputted information may be through audio (e.g., “the ultrasound images contain all necessary anatomical landmarks” or “the ultrasound images do not show the pleural line”) and/or through a graphical anatomical labels overlaid on the ultrasound images.

In some embodiments, a processing device guides a user based on a protocol (e.g., FAST, eFAST, RUSH) that requires collecting ultrasound images of multiple anatomical views. In such embodiments, the processing device may first instruct a user (e.g., using audio output) to collect ultrasound images for a first anatomical view (e.g., in a FAST exam, a cardiac view). The user may then provide a voice command identifying that the ultrasound images of the first view are collected (e.g., the user says “done”). The processing device may then instruct the user to collect ultrasound images for a second anatomical view (e.g., in a FAST exam, a RUQ view), etc. In some embodiments, a processing device may automatically determine which anatomical views are collected (e.g., using deep learning) and whether a view was missed. If an anatomical view was missed, a processing device may automatically inform the user, for example using audio (e.g., “the RUQ view was not collected”). When an anatomical view has been captured, the processing device may automatically inform the user, for example using audio (e.g., “the RUQ view has been collected”). As such, a processing device may provide feedback about what views have been and have not been collected during an ultrasound operation.

Examples of these and other methods of assisting a user to correctly place an ultrasound image device may be found in U.S. Pat. Nos. 10,702,242 and 10,628,932 and U.S. patent application Ser. Nos. 17/000,227, 16/118,256, 63/220,954, 17/031,283, 16/285,573, 16/735,019, 16/553,693, 63/278,981, 13/544,058, 63/143,699, and 16/880,272, the contents of which are incorporated by reference herein in their entireties.

In Block 255, the processing device automatically captures or receives a selection to capture one or more ultrasound images (i.e., saves to memory on the processing device or another device, such as a server) in accordance with one or more embodiments. In some embodiments, capturing ultrasound images may be performing using voice control (e.g., a user may say “Capture image” or “Capture cine for 2 seconds” or “Capture cine” and then “End capture”). In some embodiments, the processing device may automatically capture one or more ultrasound images. For example, when the quality of the ultrasound images collected by the ultrasound imaging device exceeds or meets a threshold quality, the processing device may automatically perform a capture. In some embodiments, when the quality threshold is met or exceeded, some or all of those ultrasound images for which the quality was calculated are captured. In some embodiments, when the quality threshold is met or exceeded, subsequent ultrasound images (e.g., a certain number of images, or images for a certain time span) are captured.

In Block 260, the processing device automatically completes a portion or all of an ultrasound imaging worksheet for the ultrasound imaging examination, or receives input (e.g., voice commands) from the user to complete a portion or all of the ultrasound imaging worksheet in accordance with one or more embodiments. In some embodiments, the processing device may retrieve an electronic medical record (EMR) of a patient and complete a portion or all of the ultrasound imaging worksheet based on the EMR. In some embodiments, inputs may be provided to a worksheet using voice control. For example, a user may say “indication is chest pain.” In some embodiments, the processing device may provide an audio prompt or a display prompt to a user in order to complete a portion of a worksheet. For example, the processing device may say “What are the indications?” If a user does not provide needed information through a voice interface, the processing device may provide an audio or display prompt. The processing device may transform the user's input data into a structured prose report, such as a radiology report.

In some embodiments, selections of organizations, patients, ultrasound imaging examination types, ultrasound imaging modes, ultrasound imaging presets, ultrasound imaging depths, ultrasound gain parameters, and TGC parameters are automatically populated in an ultrasound imaging worksheet. For example, after selecting a patient automatically in Block 210, patient data may be extracted and input into a worksheet accordingly. In a similar manner, the ultrasound imaging worksheet may obtain data acquired using one or more of the techniques described above in Blocks 205-220. On the other hand, a processing device may use a different technique to complete one or more portions of a worksheet. For example, in some embodiments, a deep learning technique may be used to automatically determine exam type based on ultrasound images/cines captured by a user. In some embodiments, a processing device sends a worksheet to a doctor by email or text to fill out later if a user doesn't do it at the time of the examination.

In Block 265, the processing device associates a signature with the ultrasound imaging examination in accordance with one or more embodiments. In some embodiments, a user may provide a signature using a voice command or other non-graphical interface input. For example, using voice control, a user may say “Sign the study” or the processing device may ask the user “Do you want to sign the study?” and the user may respond. In some embodiments, a user may direct a request to another user for providing attestation, such as by saying “Send to Dr. Powers for attestation.” In some embodiments, a signature is automatically provided based on a user's facial recognition, a user's fingerprint recognition, and/or a user's voice recognition. In some embodiments, a request for a signature may be transmitted to a user device later by email or text.

In Block 270, the processing device automatically uploads the ultrasound imaging examination or receives user input (e.g., voice commands) to upload the ultrasound imaging examination in accordance with one or more embodiments. For example, a processing device may upload worksheets, captured ultrasound images, and other examination data to a server in a network cloud. The upload may be performed automatically after completion of an examination workflow, such as after a user completes an attestation. The examination data may also be uploaded using voice control or one or more voice commands (e.g., a user may say “Upload study” and/or the processing device may say “Would you like to upload the study” and the user responds).

In some embodiments, examination data is stored in an archive. Archives are like folders for ultrasound examinations, where a particular archive may appear as upload destinations when saving studies on a processing device. Archives may be organized based on a selected organization, selected patient, medical specialty, or a selected ultrasound imaging device. For example, clinical scans and educational scans may be stored in separate archives. In some embodiments, a default storage location may be used for each user or each ultrasound imaging device. In some embodiments, a user may select a particular archive location using voice commands (e.g., a user may say “Use Clinical archive” and/or the processing device may say “Would you like to use the Clinical archive?” and the user may respond).

As described above, for example with reference to Table 1, the ultrasound imaging devices described herein may be universal ultrasound devices capable of imaging the whole body. The universal ultrasound device may be used together with simplified workflows specifically designed and optimized for assisting a user who may not be an expert in ultrasound imaging to perform specific ultrasound examinations. These ultrasound examinations may be for imaging, for example, the heart, lungs (e.g., to detect B-lines as an indication of congestive heart failure), liver, aorta, prostate (e.g., to calculate benign prostatic hyperplasia (BPH) volume), radius bone (e.g., to diagnose osteoporosis), deltoid, and femoral artery.

Turning to FIG. 16B, FIG. 16B shows a flowchart in accordance with one or more embodiments. Specifically, FIG. 16B describes a method for performing one or more ultrasound scans using simplified workflows. In particular, the workflow may be for an ultrasound imaging protocol that includes multiple ultrasound images or cines of different anatomies (each generally referred to herein as a scan). The blocks in FIG. 16B may be performed by a processing device (e.g., the processing device 104) in communication with an ultrasound device (e.g., the ultrasound device 102). While the various blocks in FIG. 16B are presented and described sequentially, one of ordinary skill in the art will appreciate that some or all of the blocks may be executed in different orders, may be combined or omitted, and some or all of the blocks may be executed in parallel. Furthermore, the blocks may be performed actively or passively. As will be described below, the process of FIG. 16B may be performed in conjunction with the process of FIG. 16A.

In Block 304, the processing device automatically selects a patient or receives a selection of the patient from a user in accordance with one or more embodiments. Block 304 may be the same as Block 210.

In Block 305, the processing device automatically selects an ultrasound imaging exam type or receives a selection from the user of the ultrasound imaging exam type in accordance with one or more embodiments. Block 305 may be the same to Block 220. As an example, the ultrasound imaging exam type may be a basic assessment of heart and lung function protocol (referred to herein as a PACE examination) that includes capturing multiple ultrasound images or cines of the heart and lungs. In some embodiments, a processing device may automatically select the PACE examination for all patients. As another example, the ultrasound imaging exam type may be a congestive heart failure (CHF) examination. In other words, an examination may be for a patient diagnosed with congestive heart failure (CHF) with the goal of monitoring the patient for pulmonary edema. A count of B-lines, which are artifacts in lung ultrasound images, may indicate whether there is pulmonary edema.

In Block 310, the processing device automatically selects an ultrasound imaging mode, an ultrasound imaging preset, an ultrasound depth, an ultrasound gain, and/or time gain compensation (TGC) parameters corresponding to the ultrasound imaging exam type. For example, if the PACE exam is selected and the first scan of the PACE exam is a B-mode scan of the right lung, the imaging mode may be automatically selected to be B-mode and the preset may be automatically selected to be a lung preset. As another example, if a CHF exam is selected, the imaging mode may be automatically selected to be B-mode and the preset may be automatically selected to be a lung preset. Depth, gain, and TGC optimized for imaging this particular anatomy may also be automatically selected. This automatic selection may be the same as Blocks 225, 230, 235, 240, and 245.

In Block 315, the processing device guides the user to correctly place the ultrasound imaging device on the patient for capturing one or more ultrasound images (e.g., a cine) associated with a particular scan in accordance with one or more embodiments. For example, the scan may be part of the protocol selected in Block 305. Block 315 may be the same as Block 250. The guidance may be of one or more types. In some embodiments, the guidance may include a probe placement guide. The probe placement guide may include one or more images, videos, audio, and/or text that indicate how to place an ultrasound imaging device on a patient in order to collect a clinically relevant scan. The probe placement guide may be presented before and/or during ultrasound scanning.

In some embodiments, the guidance may include a scan walkthrough during ultrasound imaging. In some embodiments, the scan walkthrough may include a real-time quality indicator that is presented based on ultrasound data in accordance with one or more embodiments. The real-time quality indicator may be automatically presented to a user using an audio device and/or a display device based on analyzing one or more captured ultrasound images. In particular, in real-time as ultrasound images are being collected, a quality indicator may indicate a quality of recent ultrasound images (e.g., the previous N ultrasound images or ultrasound images collected during the previous T seconds). A quality indicator may indicate quality based on a status bar that changes length based on changes in quality. Quality indicators may also indicate a level of quality using predetermined colors (e.g., different colors are associated with different quality levels). For example, a processing device may present a slider that moves along a colored status bar to indicate quality. In some embodiments, quality may be indicated through audio (e.g., “the ultrasound images are low quality” or “the ultrasound images have a quality score of 25%”).

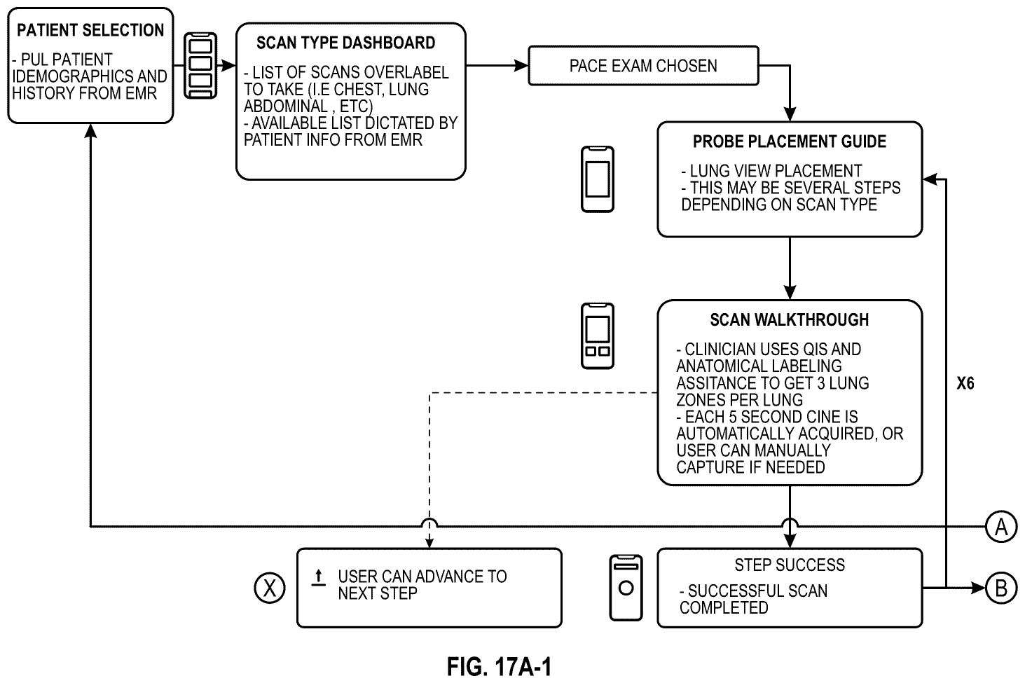

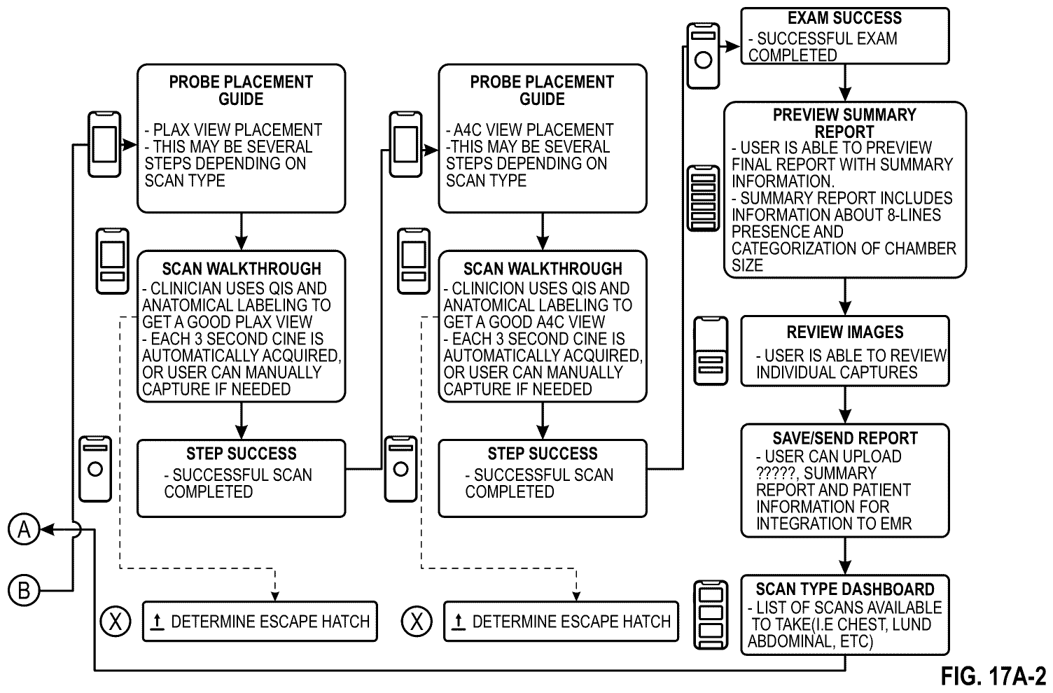

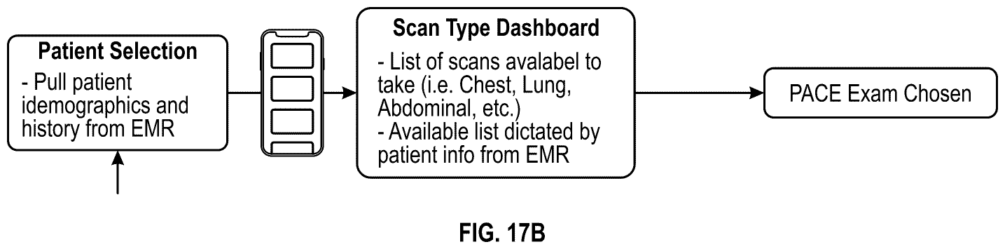

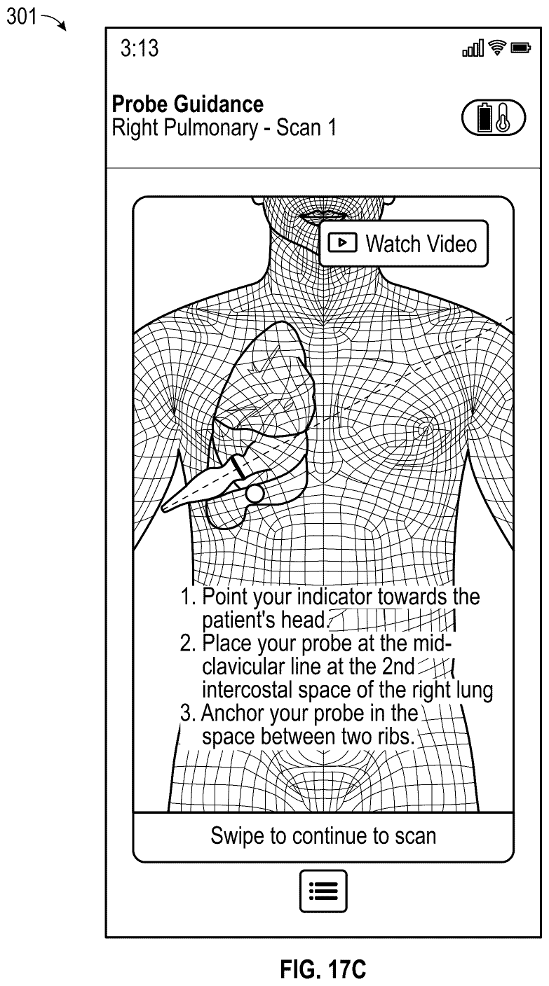

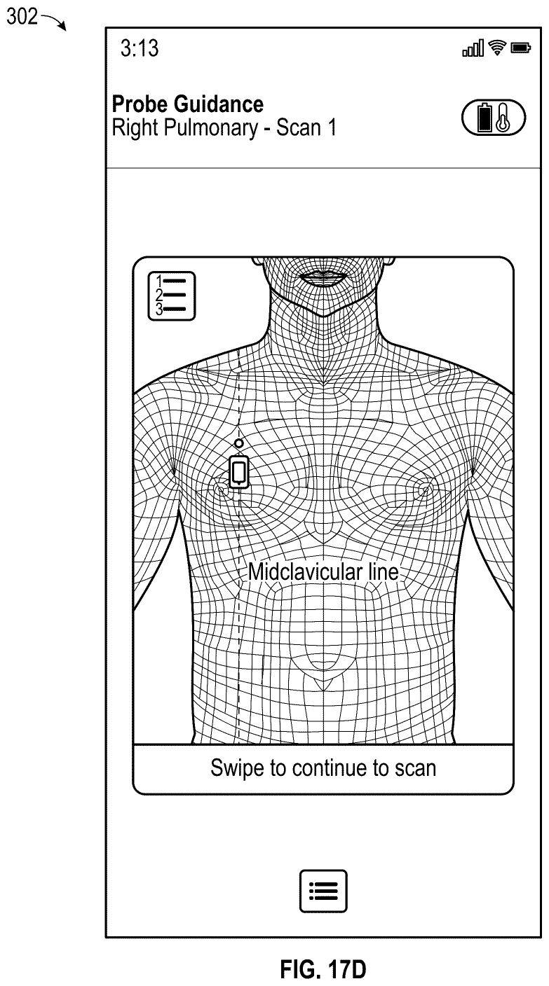

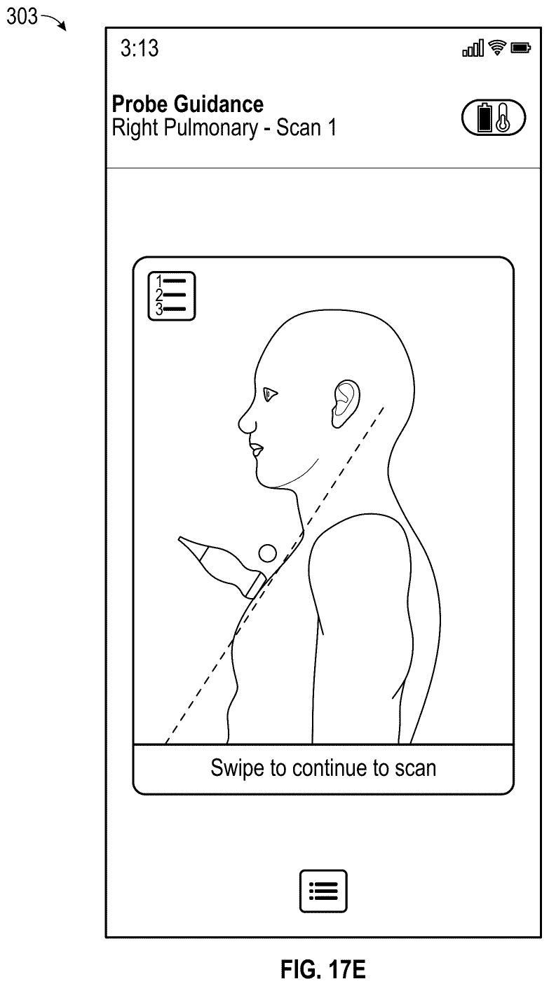

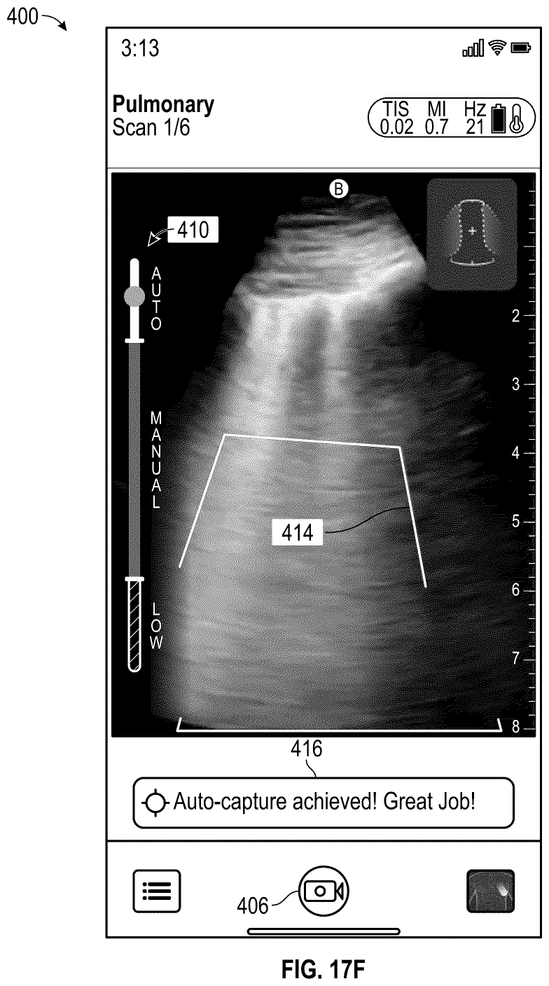

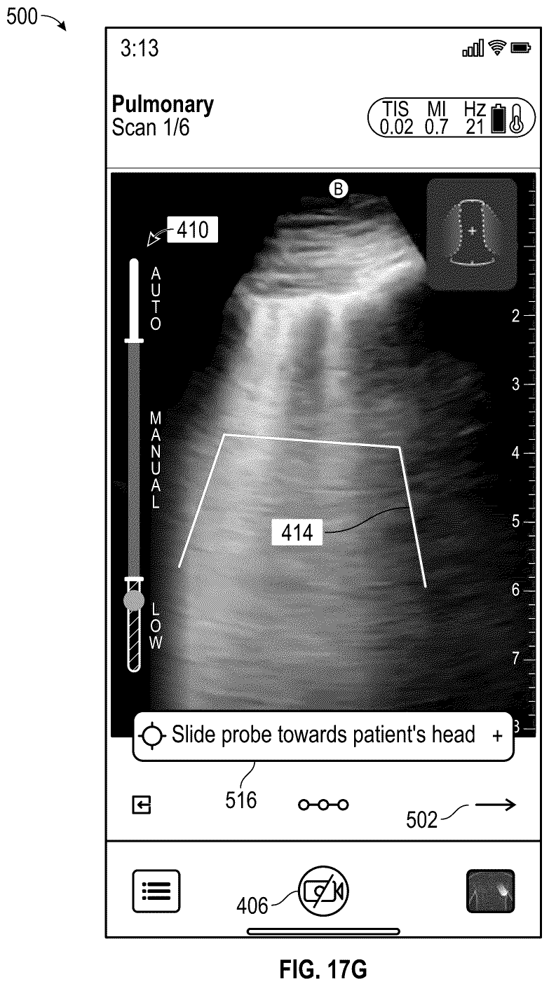

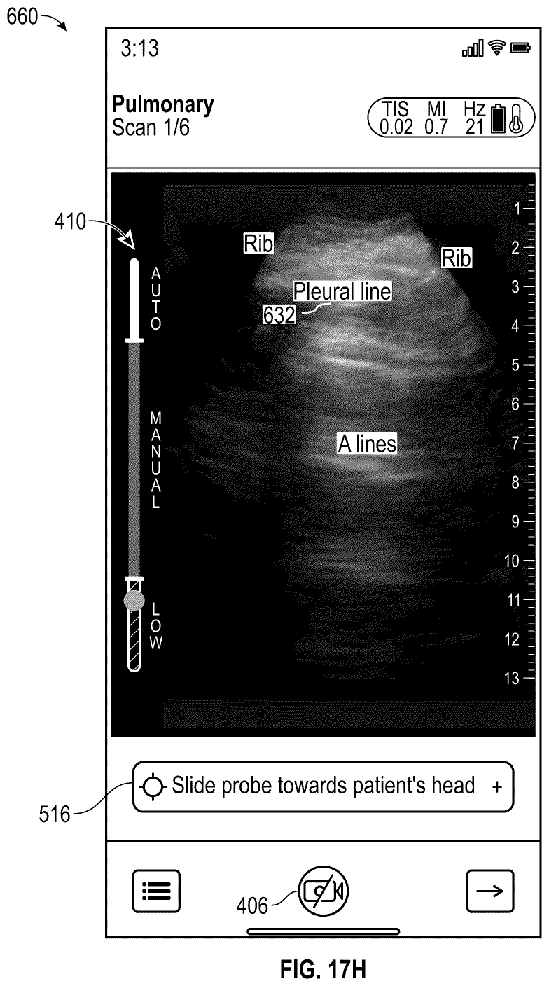

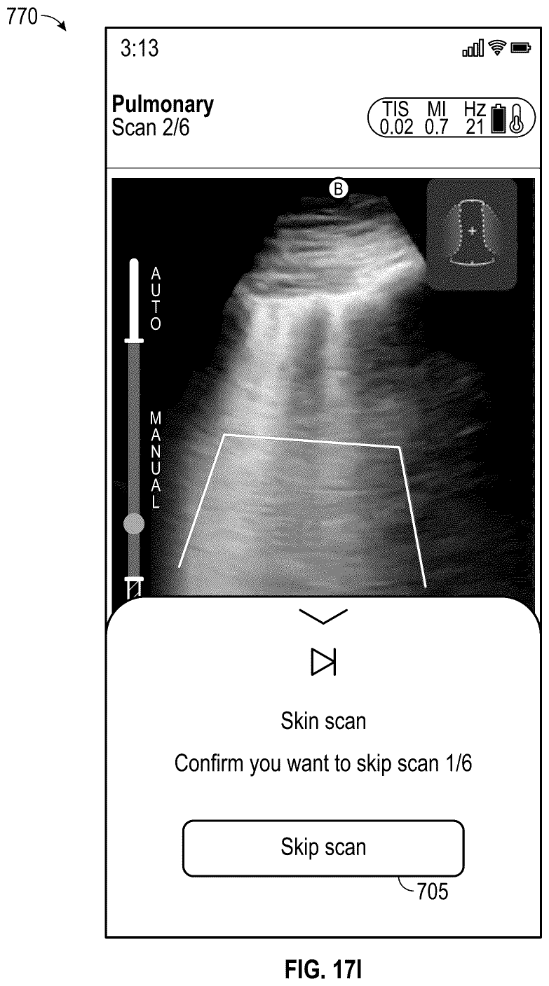

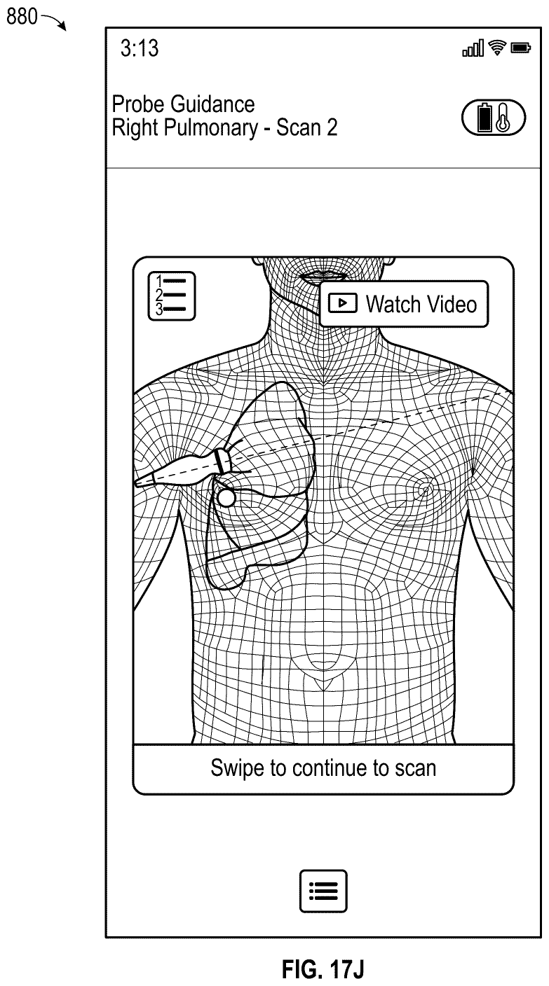

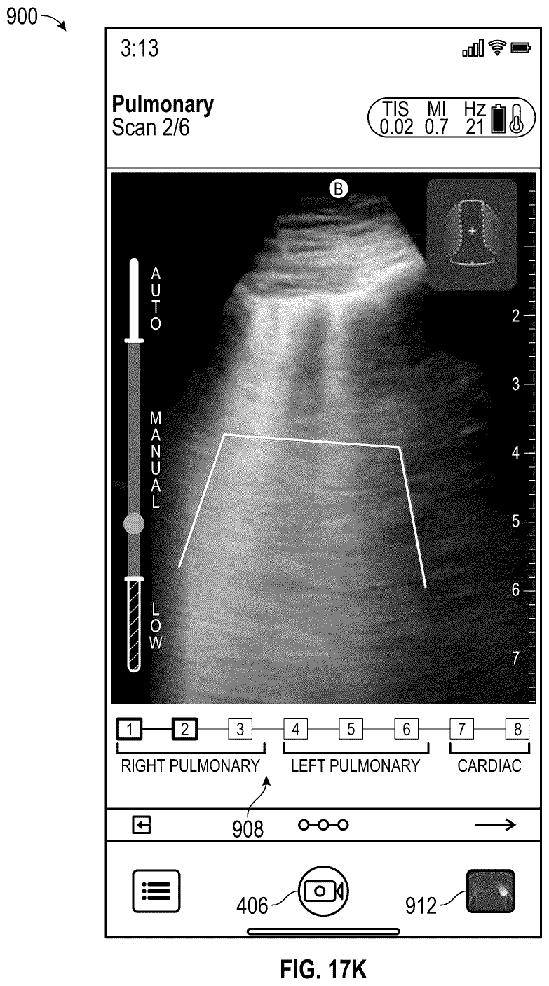

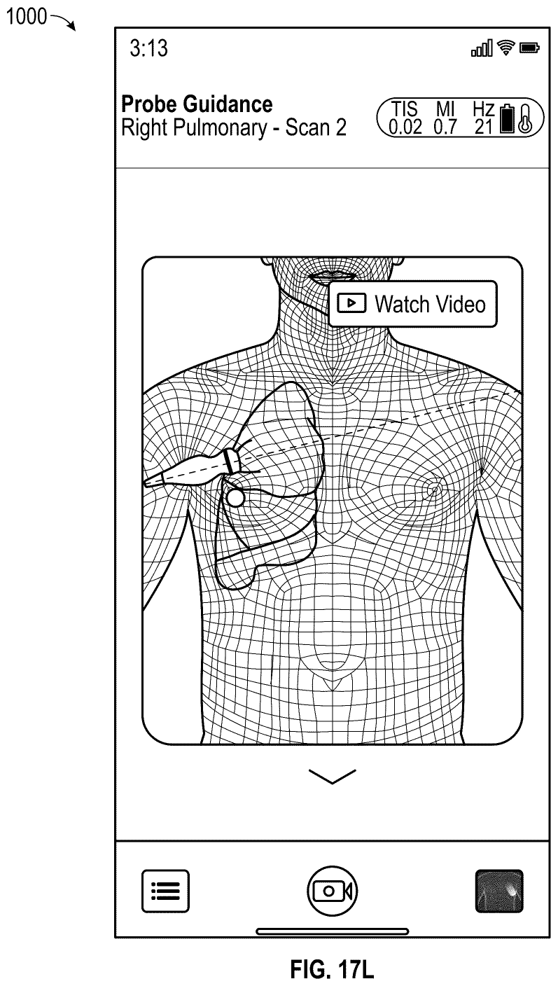

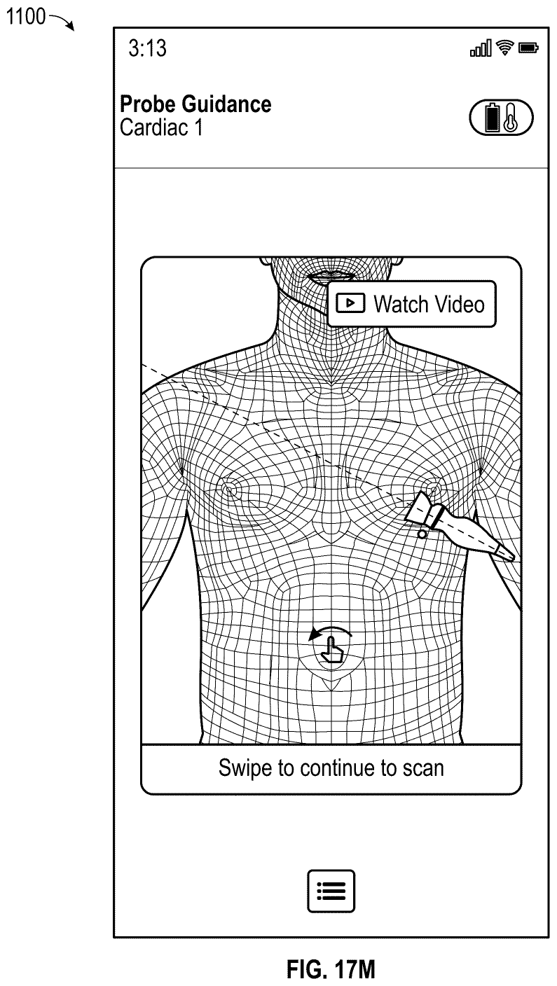

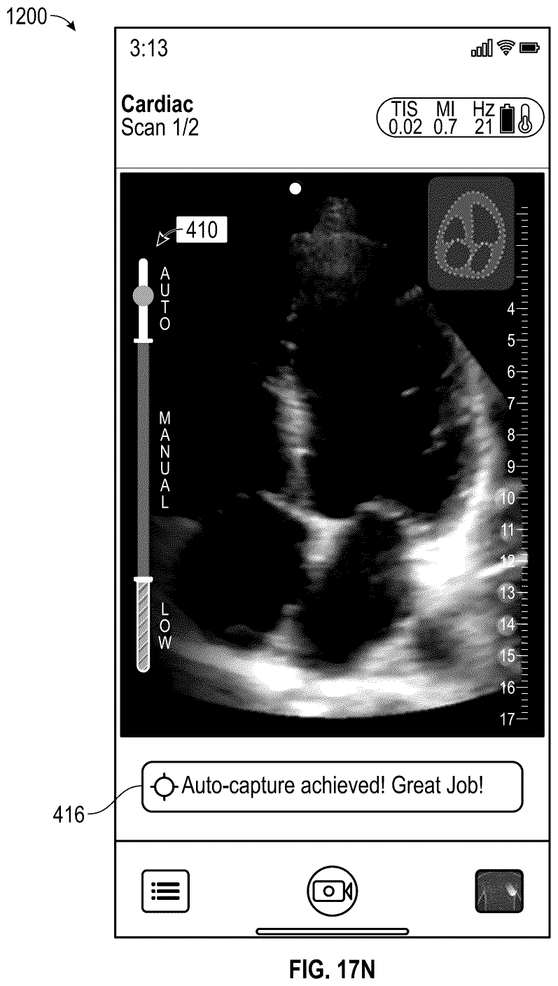

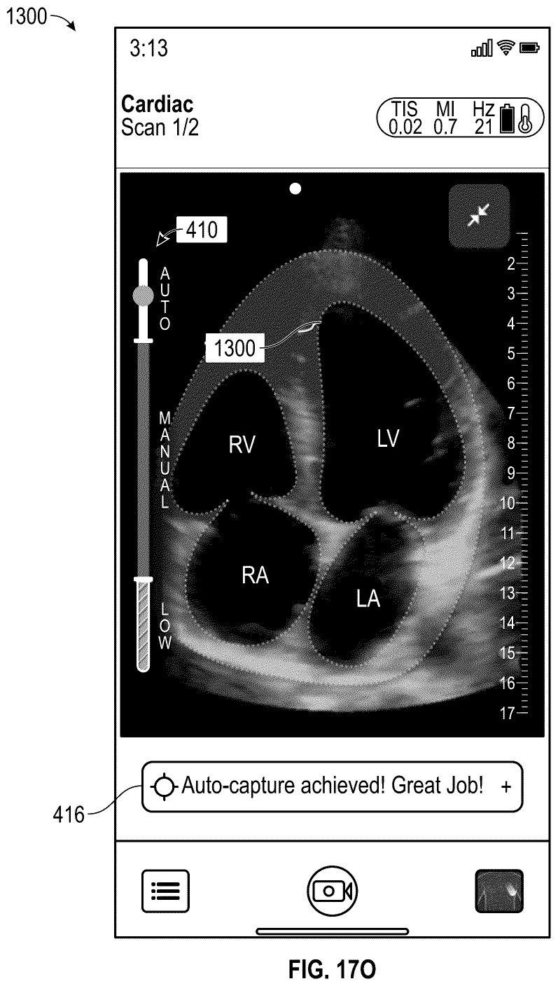

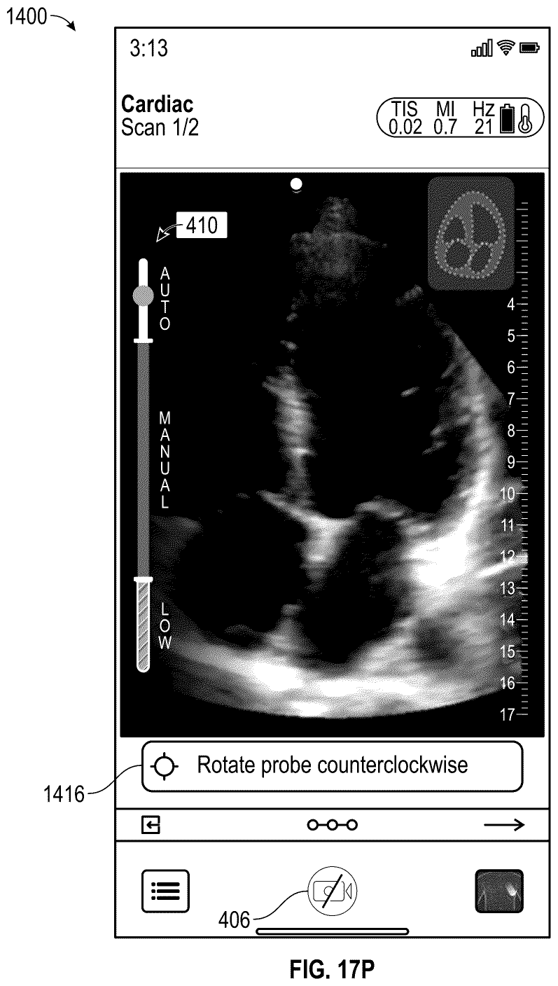

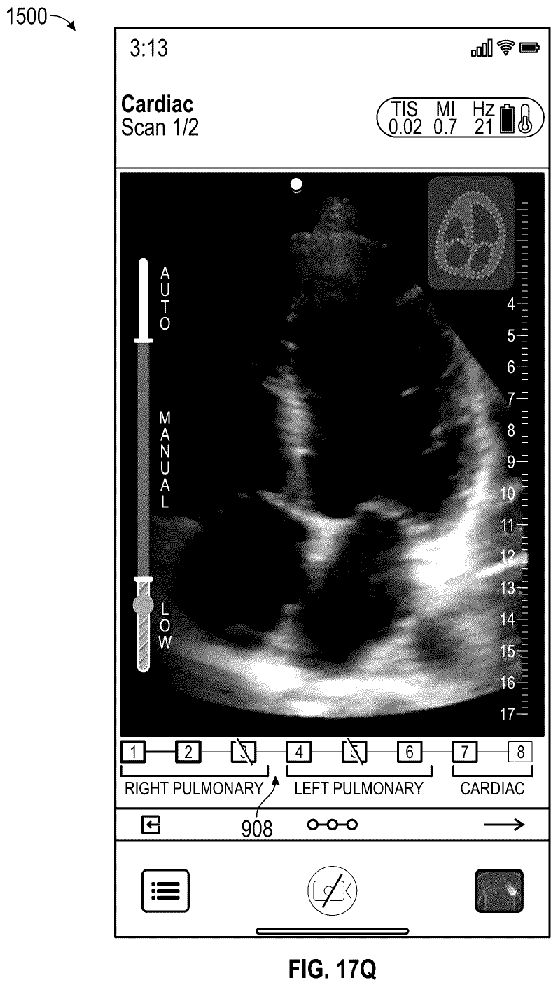

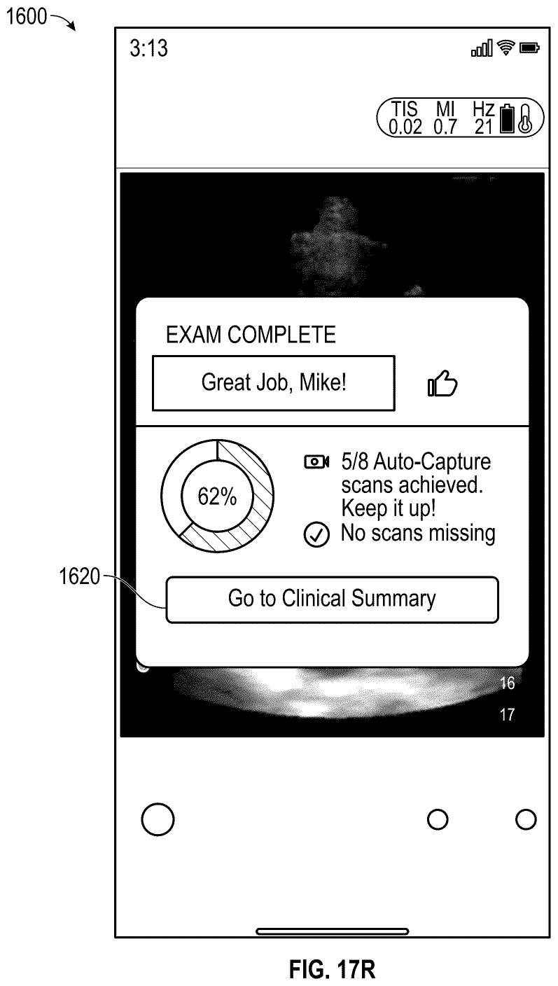

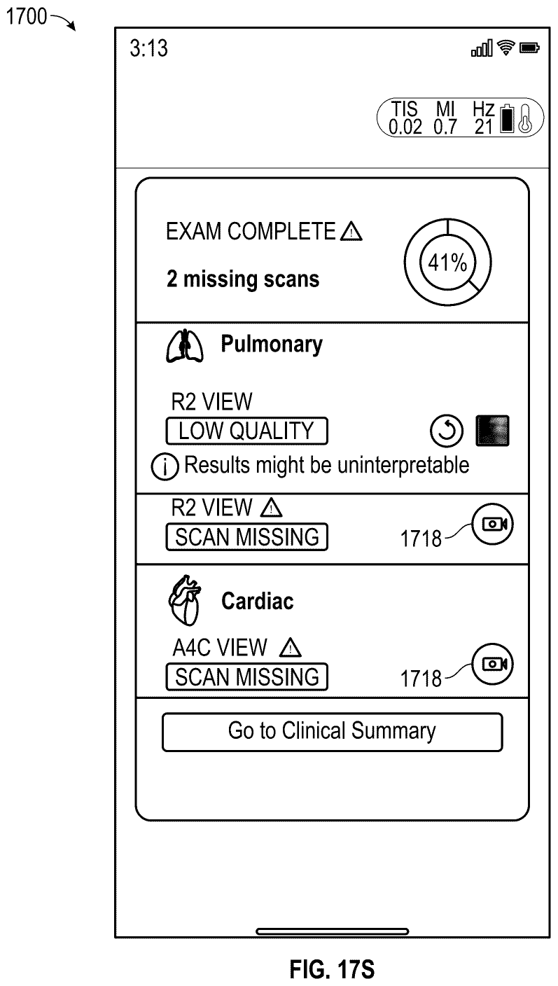

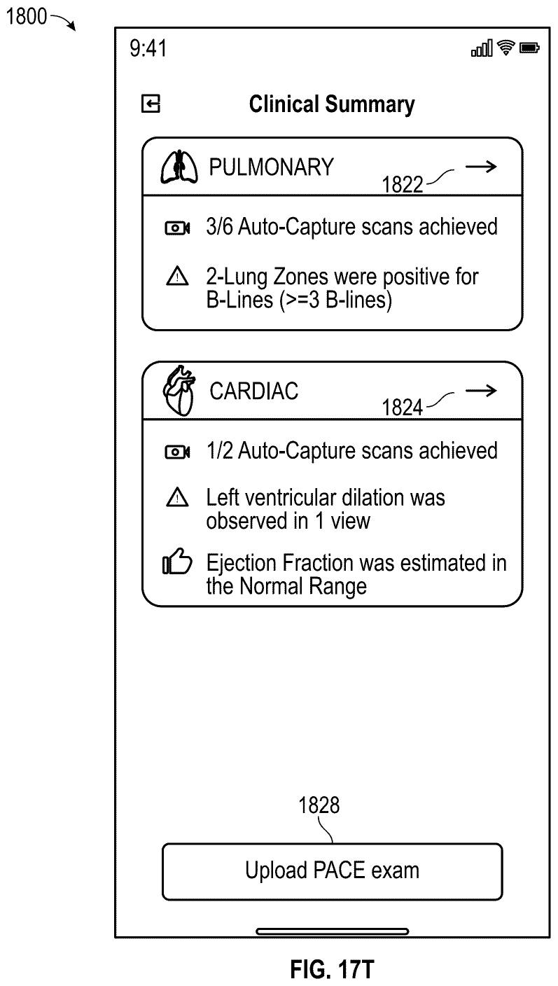

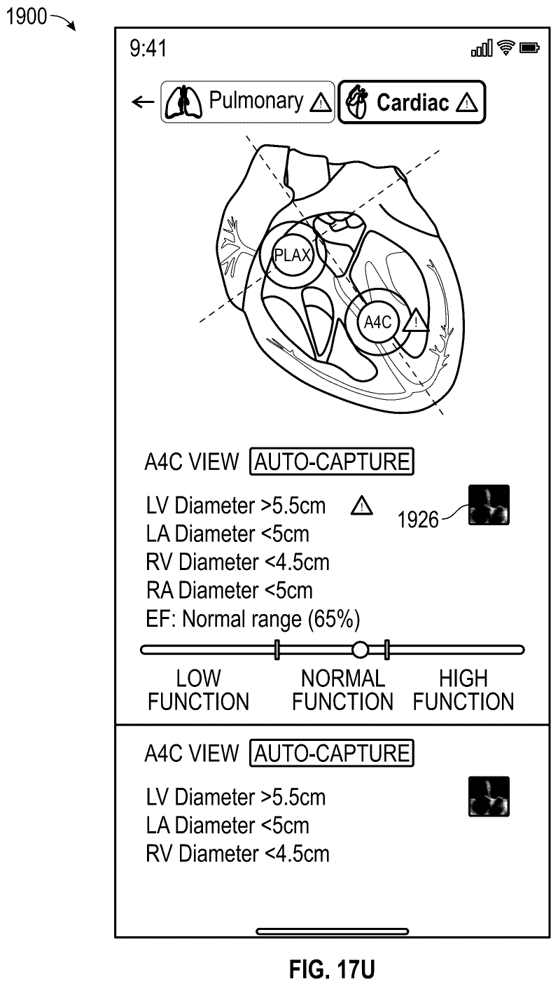

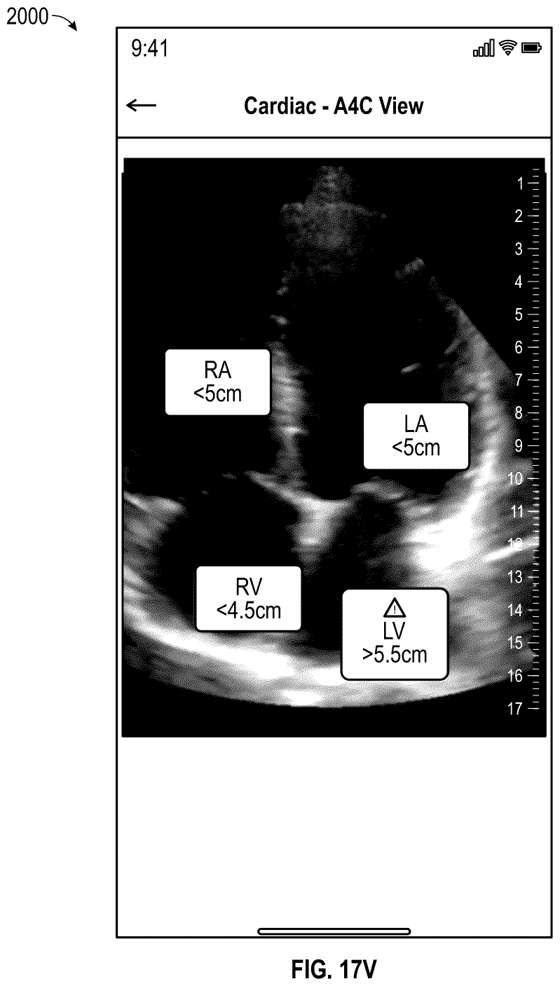

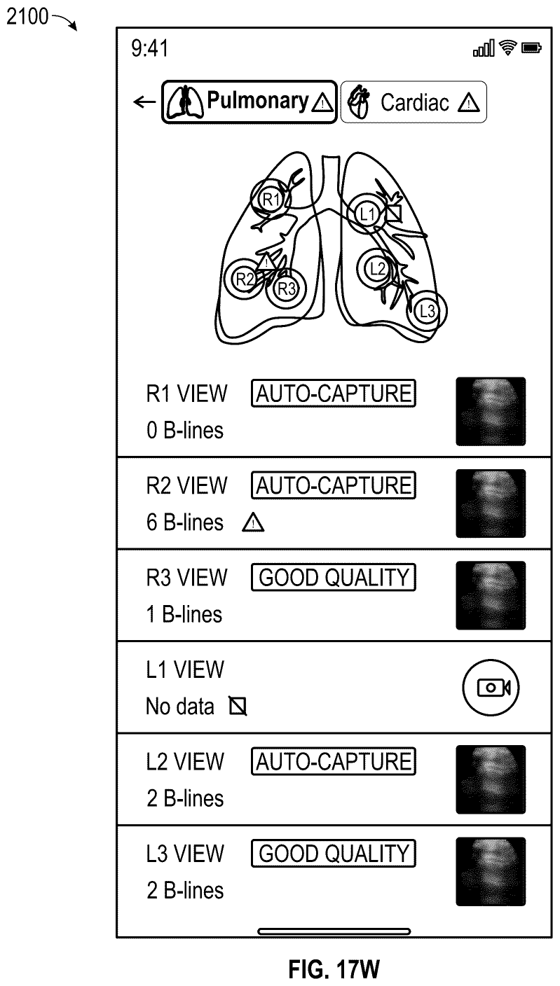

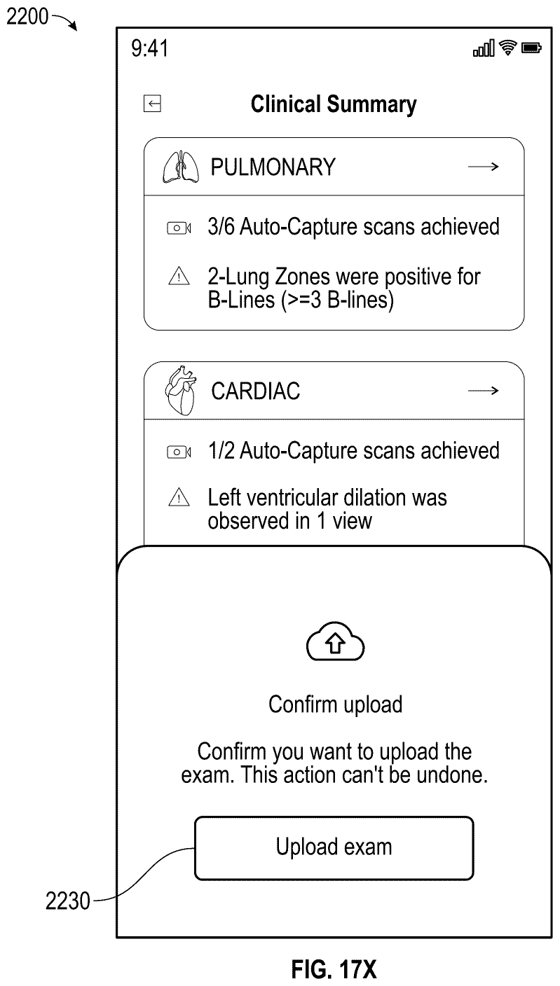

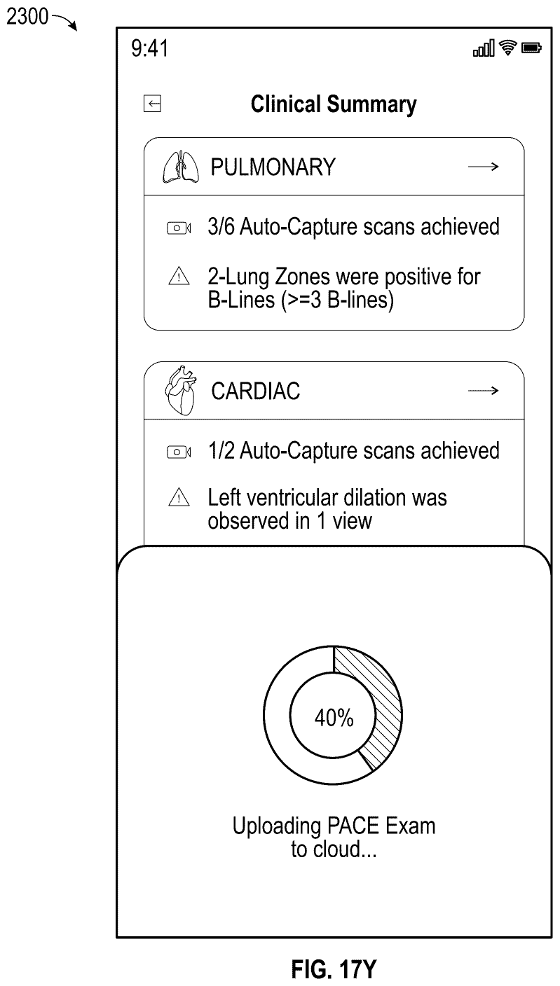

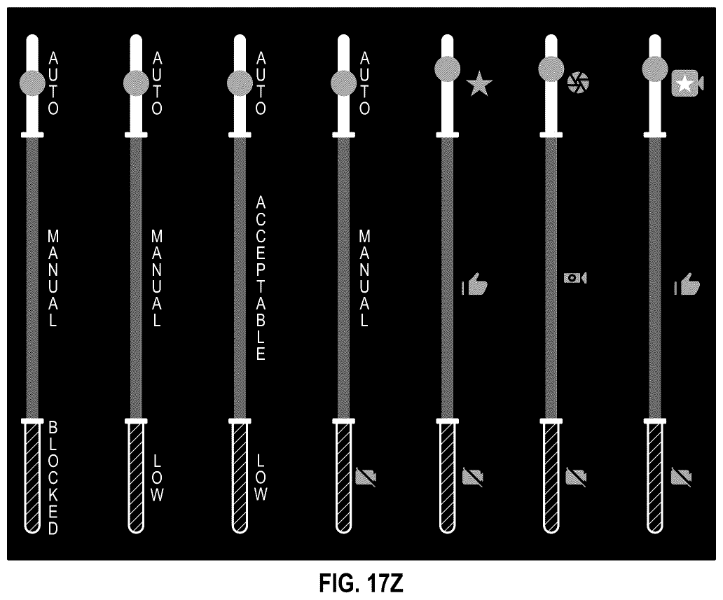

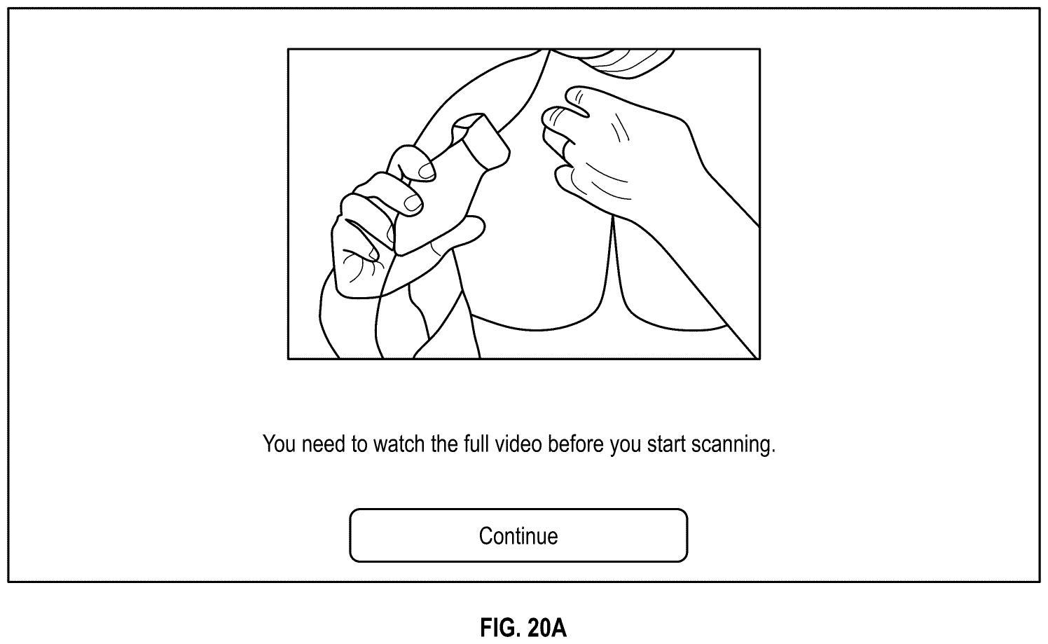

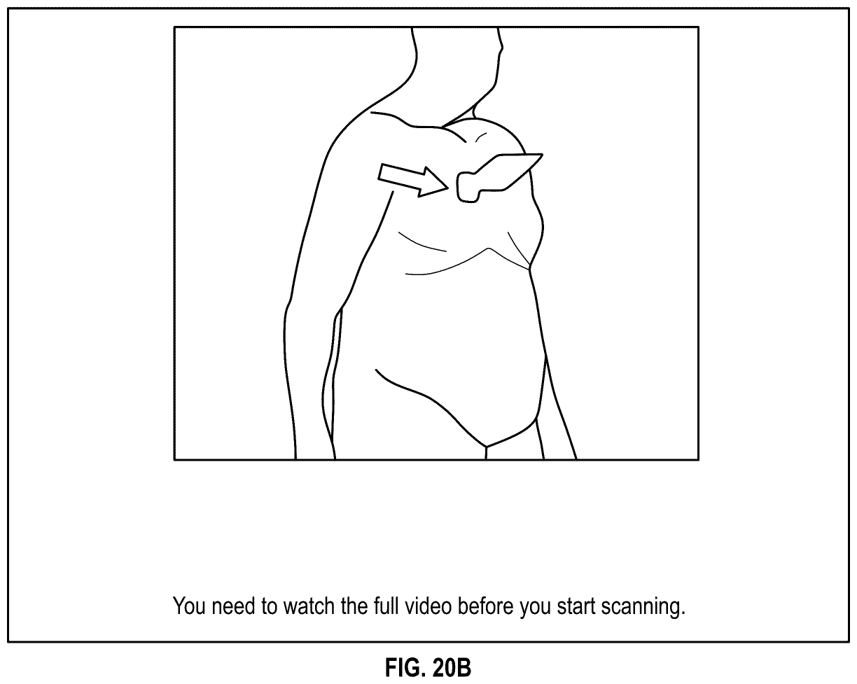

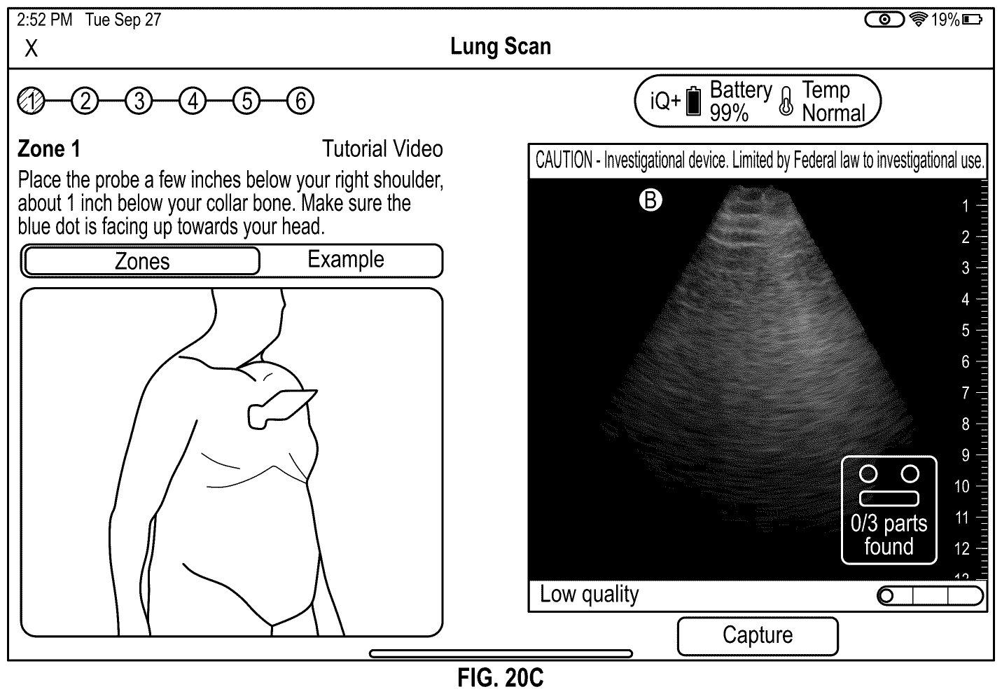

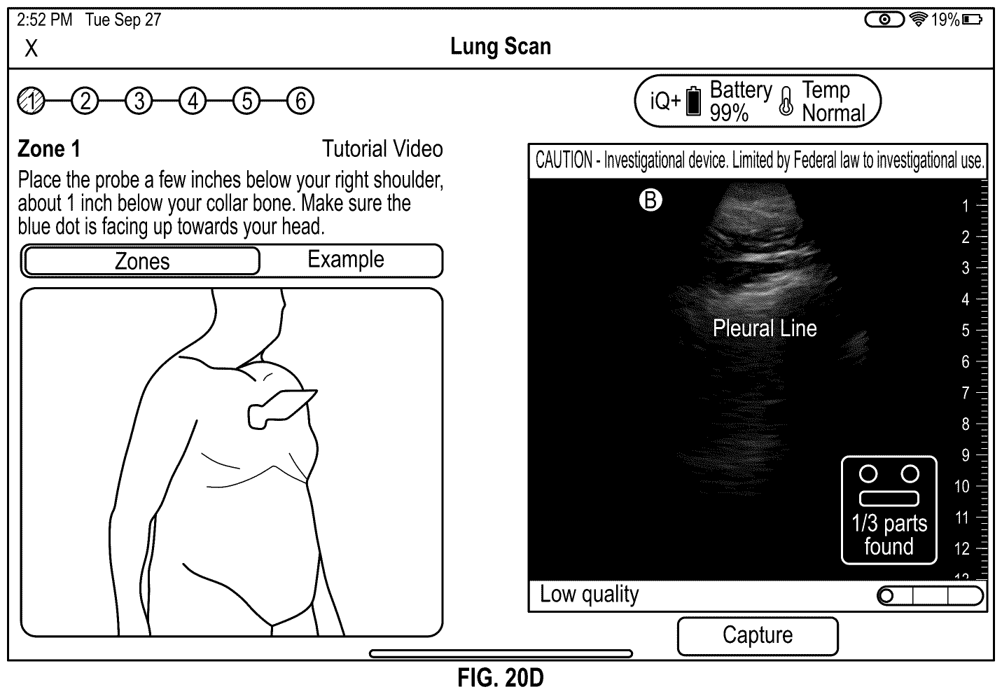

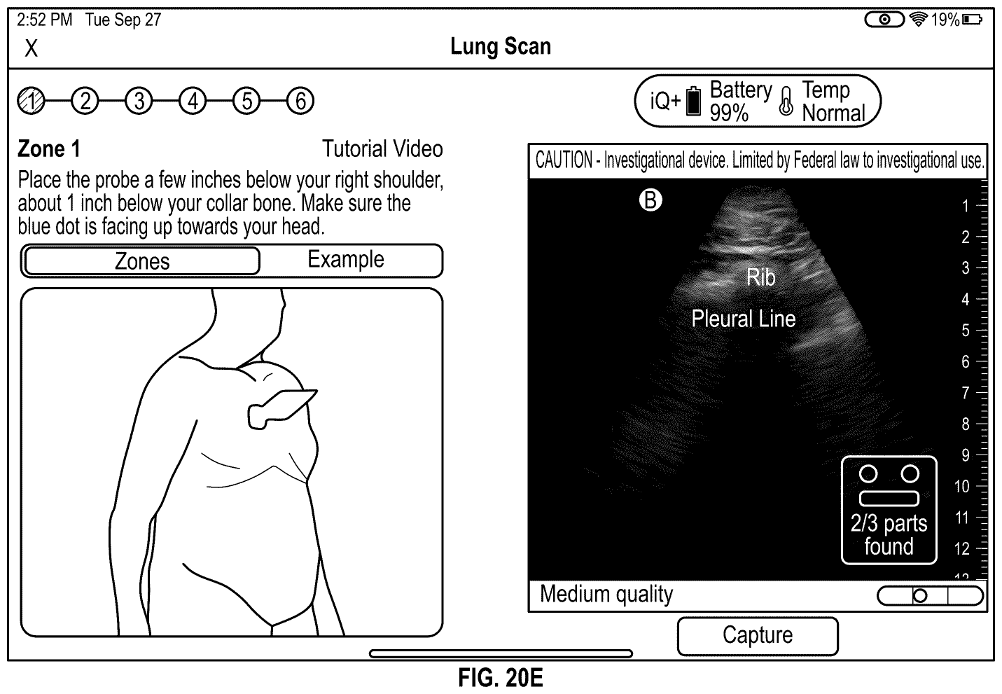

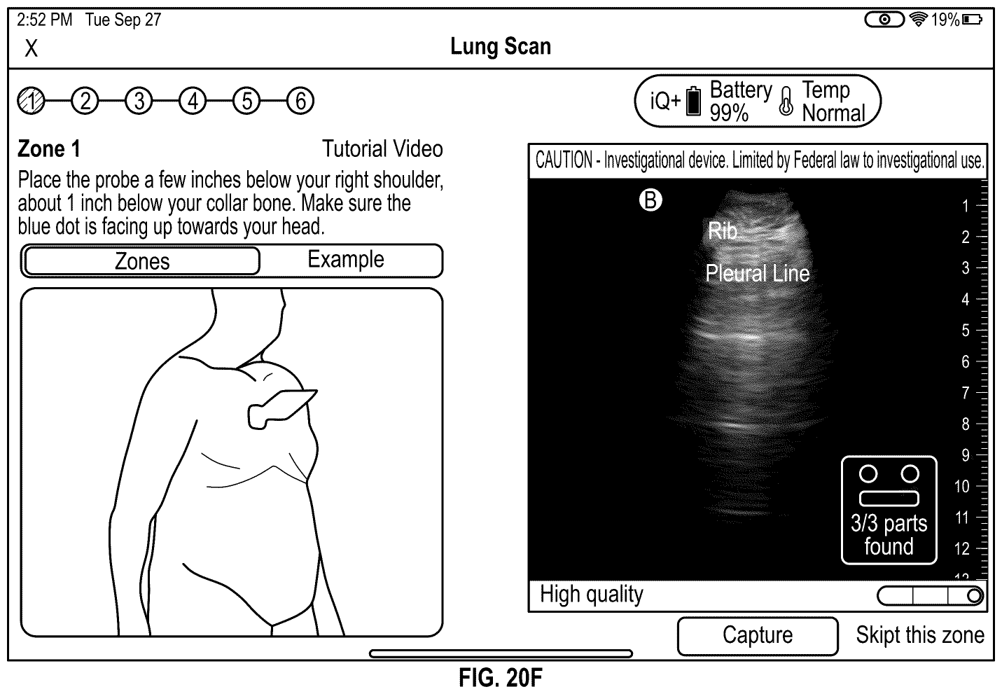

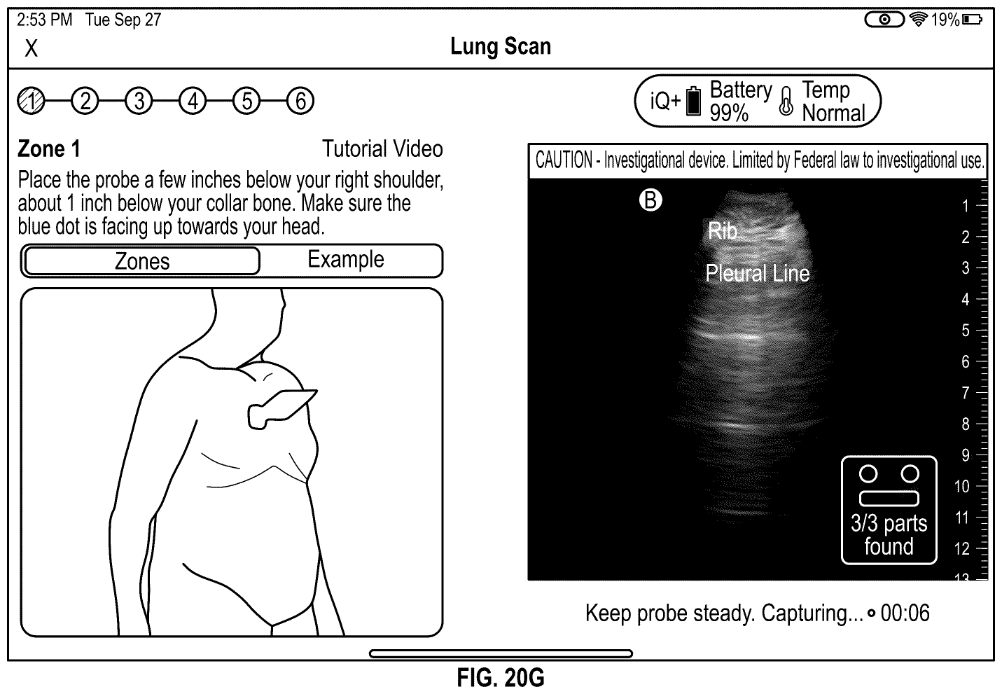

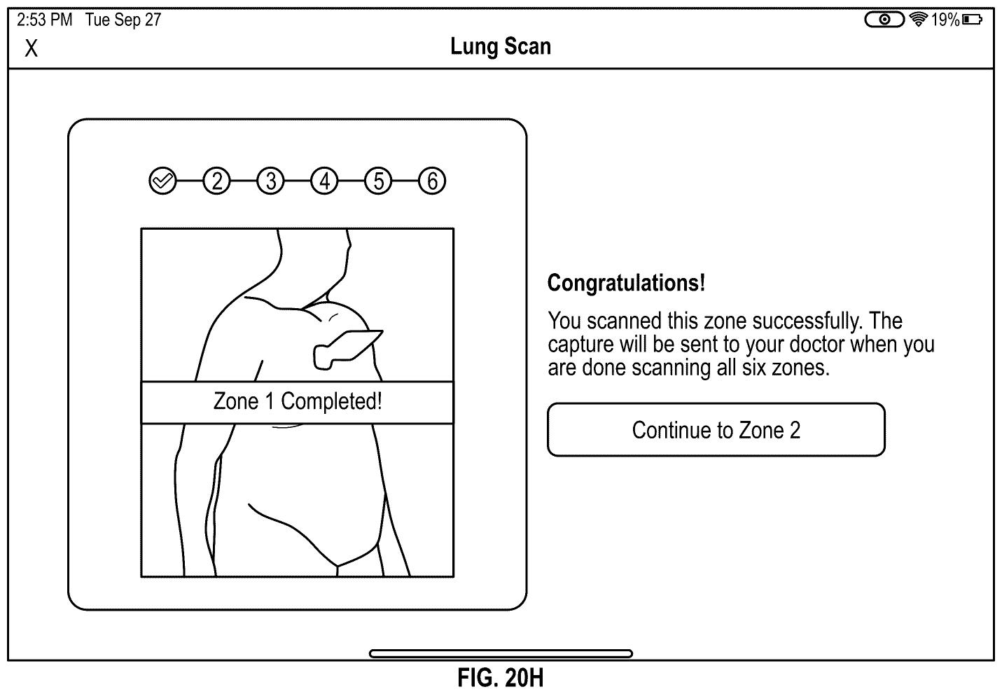

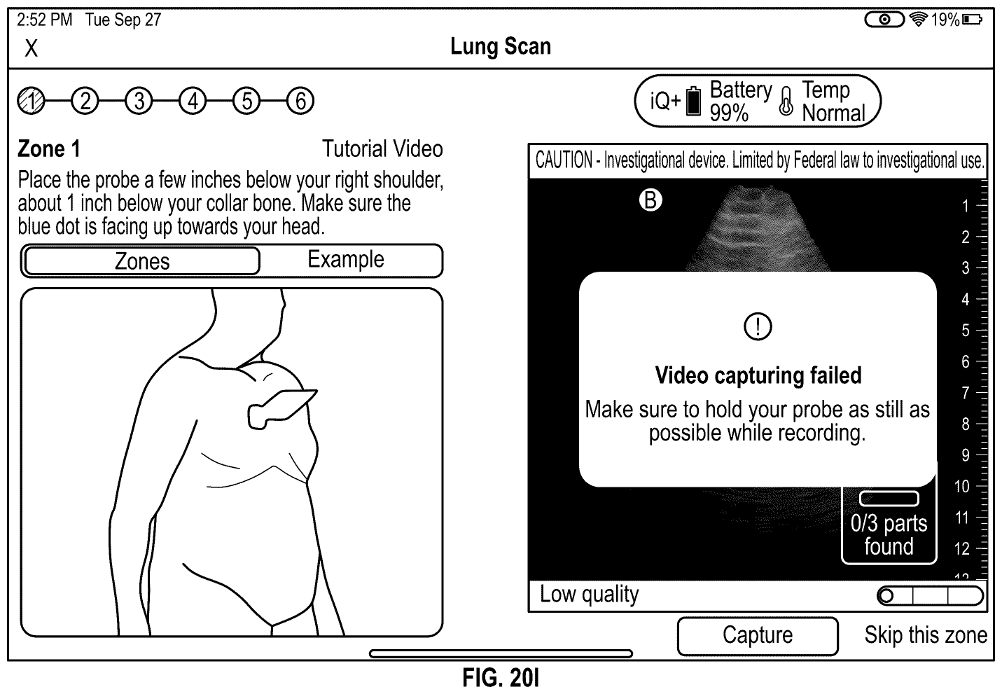

In some embodiments, the scan walkthrough may include one or more anatomical labels and/or pathological labels that are presented on one or more ultrasound images in accordance with one or more embodiments. For example, anatomical and/or pathological labeling may be performed on an ultrasound image shown on a display device. Examples of anatomical and/or pathological labeling may include identifying A lines, B lines, a pleural line, a right ventricle, a left ventricle, a right atrium, and/or a left atrium in an ultrasound image. Anatomical information may be outputted through audio (e.g., “the ultrasound images contain all necessary anatomical landmarks” or “the ultrasound images do not show the pleural line”). In some embodiments, one or more artificial intelligence techniques are used to generate the anatomical labels. Further description may also be found in U.S. patent application Ser. No. 17/586,508, the content of which is incorporated by reference herein in its entirety. FIGS. 17C-17H, 17J-17Q, and 20A-20F illustrate example GUIs that may be used in conjunction with Block 310. Other types of guidance are described further with reference to Block 250.

In Block 320, the processing device captures one or more ultrasound images (e.g., an ultrasound image or a cine of ultrasound images) associated with the particular scan in accordance with one or more embodiments. Block 320 may be the same as Block 255. A cine may be a multi-second video or series of ultrasound images. The processing device may automatically capture a cine during one or more scans during an examination based on the quality exceeding a threshold (e.g., as illustrated in FIG. 17F and FIG. 20G). In some embodiments, a cine is captured in response to voice control, such as a user saying “Capture image” or “Capture cine for 2 seconds” or “Capture cine” and then “End capture.” In some embodiments, the processing device may capture based on receiving a command from the user. For example, the user may cause a cine to be captured manually by contacting a physical button on the imaging device or an option on a GUI (e.g., the capture button 406 in the figures below). In some embodiments, a processing device may capture a six-second cine of ultrasound images of a lung, and a three-second cine of ultrasound images of a heart. In some embodiments, the processing device disables the ability to perform manual capture of an ultrasound image when a quality of recent ultrasound data does not exceed a particular threshold quality (e.g., as illustrated in FIGS. 17G and 17H). In some embodiments, during a capturing operation, the processing device may continue to monitor quality of the ultrasound images being captured. If the quality drops below a certain threshold, then the processing device may stop the capture, and may instruct the user to maintain the probe steady during capture. In some embodiments, a user may select an option to skip capturing an ultrasound image for a particular scan (e.g., as illustrated in FIG. 17I).

Upon automatic capture of an ultrasound image for a particular scan, manual capture of an ultrasound image for the particular scan, or a selection to skip capture of a particular scan, the processing device may proceed to Block 325, in which the processing device determines whether there is a next scan that is part of the protocol. For example, if in the current iteration through the workflow, the goal was to capture a scan of a first zone of the right lung, the next scan may be a second zone of the right lung. If there is a next scan, the processing device may automatically advance to guide the user to correctly place the ultrasound imaging device on the patient for capturing one or more ultrasound images associated with the next scan of the ultrasound imaging exam. In other words, the processing device proceeds back to Block 315, in which the user is guided to correctly place the ultrasound imaging device on the patient for capturing an ultrasound image or cine associated with the next scan. This is illustrated in the example automatic transition from the GUI 400 of FIG. 17F to the GUI 880 of FIG. 17J. It should be appreciated that automatically advancing to guide the user to capture the next scan may include automatically advancing to prompt the user to determine whether to proceed to capture the next scan (e.g., as illustrated in FIG. 20H).

If there is not a next scan in the protocol, the processing device proceeds to Block 330. In Block 330, the processing device presents a summary of an ultrasound imaging examination in accordance with one or more embodiments. For example, the summary may describe the exam type, subject data, user data, and other examination data, such as the date and time of an ultrasound scan. In some embodiments, a summary of the ultrasound imaging examination provides one or more scores (e.g., based on quality or other ultrasound metrics), a number of scans completed, whether or not the scans were auto-captured or manually captured, an average quality score for the scans, and which automatic calculations were calculated. FIGS. 17R-17T illustrates example GUIs 1600, 1700, and 1800 for displaying a summary. A summary may also be shown at periodic intervals during an examination, such as to display progress through various scans of an examination.

In Block 340, the processing device provides an option (e.g., the options 1822 and 1828 in FIG. 17T) for a user to review one or more captured ultrasound images or cines from one or more scans during an ultrasound imaging examination in accordance with one or more embodiments. FIGS. 17U-17W illustrate example GUIs 1900, 2000, 2100 for providing review of ultrasound images or cines.

Turning to FIG. 17A, FIG. 17A shows a flowchart in accordance with one or more embodiments. Specifically, FIG. 17A describes a method for performing a PACE exam using simplified workflows. The method of FIG. 17A may be an implementation of the method of FIG. 16B specifically for a PACE exam. The blocks in FIG. 17A may be performed by a processing device (e.g., the processing device 104) in communication with an ultrasound device (e.g., the ultrasound device 102). While the various blocks in FIG. 17A are presented and described sequentially, one of ordinary skill in the art will appreciate that some or all of the blocks may be executed in different orders, may be combined or omitted, and some or all of the blocks may be executed in parallel. Furthermore, the blocks may be performed actively or passively.

A PACE exam may include lung and heart scans. The lung scans may include 6 scans, 1 scan for each of 3 zones of each of the 2 lungs. The heart scans may include 2 scans, one for parasternal long axis (PLAX) view and one for apical four-chamber (A4C) view.

The method of FIG. 17A begins with patient selection, which may be the same as Block 300 as is highlighted in FIG. 17B. The method proceeds to selection of scan type (in this method, a PACE exam), which may be the same as Block 305. The method then proceeds to presentation of a probe placement guide for the first lung scan and then a scan walkthrough for this scan, including a presentation of a quality indicator and anatomical labels. The probe placement guide, quality indicators, and anatomical labels may be part of Block 315. A six-second long cine is captured for each lung scan, and capture may occur automatically or manually. The capturing step be the same as Block 320. Once the first lung scan has been successfully captured, the method automatically advances to the next lung scan, or in other words, the method goes back to present a probe placement guide for the second lung scan, a scan walkthrough for this scan, and capture of this lung scan. These steps are repeated until all six lung scans have been captured (or skipped), after which the method proceeds to heart scans.

The method then proceeds to presentation of a probe placement guide for the first heart scan (in the example of FIG. 17A, a PLAX view) and then a scan walkthrough for this scan, including a presentation of a quality indicator and anatomical labels. The probe placement guide, quality indicators, and anatomical labels may be part of Block 315. A two-second long cine is captured for each heart scan, and capture may occur automatically or manually. The capturing step be the same as Block 320. Once the first heart scan has been successfully captured, the method automatically advances to the next heart scan, or in other words, the method goes back to present a probe placement guide for the second heart scan (in the example of FIG. 17A, an A4C view), a scan walkthrough for this scan, and capture of this heart scan.

Once all scans of the PACE exam have been successfully captured, the method automatically advances to provide a summary report, which may include information about B-line presence and categorization of chamber size. The user may also be able to review images from individual images. Then, the user can upload the captures, summary report, and other information such as patient information.

FIGS. 17C-17Z provide some examples of graphical user interfaces (GUIs) associated with a PACE examination workflow for some embodiments. Any details or features shown in a GUI in the context of one scan may be included in the GUIs for any of the scans.

FIG. 17C illustrates a GUI 301 including a probe placement guide. In the example of FIG. 17C, GUI 300 is the start of the PACE exam workflow, the start of the pulmonary workflow, and the start of the workflow for collecting scan 1 for the right lung. FIGS. 17D and 17E illustrate alternative example probe placement guides in GUIs 302 and 303, respectively. The GUIs of FIGS. 17C-17E may be shown before ultrasound imaging begins. Upon swiping, or after expiration of a timer, the processing device proceeds to exam GUIs 400, 500, or 660 of FIG. 17F, 17G, or 17H, respectively.

FIG. 17F illustrates a GUI 400 that may be shown during ultrasound imaging. Lung ultrasound images are shown in real time. Quality indicator 410 shows three ranges. When quality reaches the highest range, the system auto-captures a 6-second cine and provides a text indication 416 of the auto-capture. B-lines 414 are optionally identified and highlighted in real-time (representing an example of real-time pathology detection). Upon auto-capture or manual capture (i.e., the user selecting capture button 406), the processing device automatically advances to GUI 880 of FIG. 17J to repeat the capture process for the next scan in the pulmonary portion of the PACE protocol, namely scan 1 of the right lung.

FIG. 17G illustrates a GUI 500 that may be shown during ultrasound imaging. Lung ultrasound images are shown in real time. If, unlike in FIG. 17F, quality is in the lowest range, the capture button 406 is deactivated so that manual capture cannot be performed. Text instruction 516 for moving the probe to capture a higher quality image is provided. When option 502 is selected, the processing device proceeds to GUI 770 of FIG. 17I.

FIG. 17H illustrates a GUI 660 that is an alternative to the GUI 500 of FIG. 17G. may be shown during ultrasound imaging. When quality is in the lowest range, the capture button 406 is struck through to indicate that manual capture should not be performed, but user still can perform manual capture. Real-time anatomical labeling (e.g., pleural line labeling 632) in the ultrasound image may assist user with probe placement.

FIG. 17I illustrates a GUI 770 allows a user to skip a scan. When option 705 is selected, proceed to GUI 880.

FIG. 17J illustrates a GUI 880 that is the start of the workflow for collecting scan 2 for the right lung. GUI 880 depicts guidance for collecting scan 2 for the right lung. Upon swiping, or after expiration of a timer, proceed to GUI 900 of FIG. 17K.

FIG. 17K illustrates a GUI 900 that may be shown during ultrasound imaging. Lung ultrasound images are shown in real time. When quality is in the middle range, the user may manually capture a 6-second cine by selecting the capture button 406. GUI 900 depicts an optional progress bar 908 (which may be present in any of the above GUIs) indicating progress through the PACE workflow. Upon selection of guidance indicator 912, proceed to GUI 1000 of FIG. 17L.

FIG. 17L illustrates a GUI 1000 that depicts guidance for collecting scan 2 for the right lung.

Upon completion of the pulmonary workflow, the processing device depict GUI 1100 of FIG. 17M. FIG. 17M illustrates the GUI 1100 that is the start of the cardiac workflow and the start of the workflow for collecting scan 1 for the heart. GUI 1100 depicts guidance for collecting scan 1 for the heart. Upon swiping, or after expiration of a timer, proceed to exam GUIs 1200, 1300, 1400, or 1500.

FIG. 17N illustrates the GUI 1200 in which cardiac ultrasound images are shown in real time. When quality reaches the highest range, the system auto-captures a 3-second cine and provides a text indication 416 of the auto-capture. Upon auto-capture or manual capture, the system automatically advances to the next GUI to repeat the capture process for the next scan in the cardiac portion of the PACE protocol.

FIG. 17O illustrates the GUI 1300 that is an alternative to GUI 1200. Real-time anatomical labeling (e.g., left ventricle labeling 1332) in the ultrasound image may assist user with probe placement.

FIG. 17P illustrates the GUI 1400. When quality is in the lowest range, the capture button 406 is deactivated so that manual capture cannot be performed. Text instruction 1416 for moving the probe are provided.

FIG. 17Q illustrates the GUI 1500 which depicts the optional progress bar 908 indicating progress through the PACE workflow, which scans were completed, and which scans were not.

At the completion of the PACE exam workflow, if all the scans were achieved, the processing device shows GUI 1600 of FIG. 17R. GUI 1600 depicts user feedback. Inputs to the score may include number of scans completed; whether or not they were auto-captured; if manual capture, what was the average quality score; and which of the automatic interpretations were able to be provided. Upon selection of clinical summary option 1620, the processing device proceeds to GUI 1800 of FIG. 17T.

At the completion of the PACE exam workflow, if some the scans were not achieved, GUI 1700 of FIG. 17S is shown. GUI 1700 depicts information about missing scans and low quality scans. Upon selection of the rescan options 1718, return to exam GUIs to complete these scans.

FIG. 17T illustrates GUI 1800 which depicts a clinical summary. Upon selection of pulmonary option 1822, proceed to GUI 2100 of FIG. 17W. Upon selection of cardiac option 1824, proceed to GUI 1900 of FIG. 17U. Upon selection of the upload option 1828, proceed to GUI 2200 of FIG. 17X.

FIG. 17U illustrates GUI 1900 which depicts an illustration of a heart and accompanying details for the cardiac scans, such as left ventricle (LV) diameter, left atrium (LA) diameter, right ventricle (RV) diameter, right atrium (RA) diameter, and ejection fraction (EF). Upon selection of the scan detail option 1926, proceed to GUI 2000 of FIG. 17V.

FIG. 17V illustrates GUI 2000 which depicts an illustration of lungs, and accompanying details for a particular scan.

FIG. 17W illustrates GUI 2100 which depicts details for the pulmonary scans, including B-line counts. Scan detail options may be selected to show details for particular scan in a similar manner as described above.

FIG. 17X illustrates GUI 2200 which asks for user confirmation to upload the exam. Upon selection of the upload option 2230, the processing device proceeds to GUI 2300 of FIG. 17Y.

FIG. 17Y illustrates GUI 2300 which depicts progress of the PACE exam upload.

FIG. 17Z illustrates alternatives for the quality indicator 410.

FIGS. 20A-20I provide some examples of graphical user interfaces (GUIs) associated with a CHF examination workflow for some embodiments.

FIG. 20A illustrates a GUI including a probe placement guide for a first scan in the CHF examination workflow. In the example of FIG. 20A, the probe placement guide includes a video.

FIG. 20B illustrates a GUI including another probe placement guide for the first scan in the CHF examination workflow. In the example of FIG. 20B, the probe placement guide includes an animation.

FIG. 20C illustrates a GUI that may be shown during ultrasound imaging. Lung ultrasound images are shown in real time. A quality indicator indicates graphically and textually three quality ranges. In the example of FIG. 20C, the quality indicator indicates low quality. An anatomical landmark indicator indicates how many landmarks may be necessary or suggested for a high-quality image are present. In the example of the CHF examination workflow, the three landmarks are the pleural line and two ribs. The anatomical landmark indicator also schematically illustrates the relative locations of the three landmarks in lung ultrasound images. In particular, the ribs are generally at the top of an ultrasound image on the right and left sides, and the pleural line is below the ribs in the middle of the ultrasound image. When a landmark is present, the anatomical landmark indicator may fill in the corresponding landmark in the schematic. The GUI also includes a progress bar indicating progress through the CHF examination workflow. In the example of FIG. 20C, the first scan of six scans is in progress. The GUI also includes a probe placement guide. In the example of FIG. 20C, the probe placement guide is an animation.

FIG. 20D illustrates a GUI that may be shown during ultrasound imaging. The GUI of FIG. 20D is the same as the GUI of FIG. 20C, except that the current ultrasound image includes one landmark, the pleural line. The pleural line is highlighted in the ultrasound image. Further description of highlighting anatomical landmarks in ultrasound images may be found in U.S. patent application Ser. No. 17/586,508, the content of which is incorporated herein by reference. The anatomical landmark indicator indicates that one landmark is present in the current ultrasound image.

FIG. 20E illustrates a GUI that may be shown during ultrasound imaging. The GUI of FIG. 20E is the same as the GUI of FIG. 20D, except that the current ultrasound image includes two landmarks, the pleural line and one rib, and the quality indicator indicates that the quality is medium. The pleural line and the rib are highlighted in the ultrasound image. The anatomical landmark indicator indicates that two landmarks are present in the current ultrasound image.

FIG. 20F illustrates a GUI that may be shown during ultrasound imaging. The GUI of FIG. 20F is the same as the GUI of FIG. 20E, except that the current ultrasound image includes three landmarks, the pleural line and two ribs, and the quality indicator indicates that the quality is high. The pleural line and the ribs are highlighted in the ultrasound image. The anatomical landmark indicator indicates that three landmarks are present in the current ultrasound image.

FIG. 20G illustrates a cine lasting six seconds being automatically captured. The capture may be automatically triggered once the quality reaches high (e.g., as in FIG. 20F). Capture may also be triggered manually using the capture button that is illustrated in the GUIs of FIGS. 20C-20F.

Once the cine for the first scan has been captured, the workflow may automatically proceed to the next scan in the workflow, as illustrates in the GUI of FIG. 20H. The GUI of FIG. 20H requires a user to select to continue to the next scan. Once the option to continue has been selected, the workflow may continue to probe placement guides like the ones of FIGS. 20A-20B, but for the second scan in the workflow. In other embodiments, user selection to continue to the next scan may not be required, and the workflow may automatically progress to the next scan. The GUI of FIG. 20H also has a progress bar indicating that the first scan in the workflow has been completed.

In some embodiments, during capture (e.g., as in FIG. 20G), the system may continue to monitor quality of the ultrasound images being captured. If the quality drops below a certain threshold (e.g., into the low quality range or the medium quality range), then the capture may stop as illustrated in the GUI of FIG. 20I. In this GUI, the user is instructed to maintain the probe steady during capture.

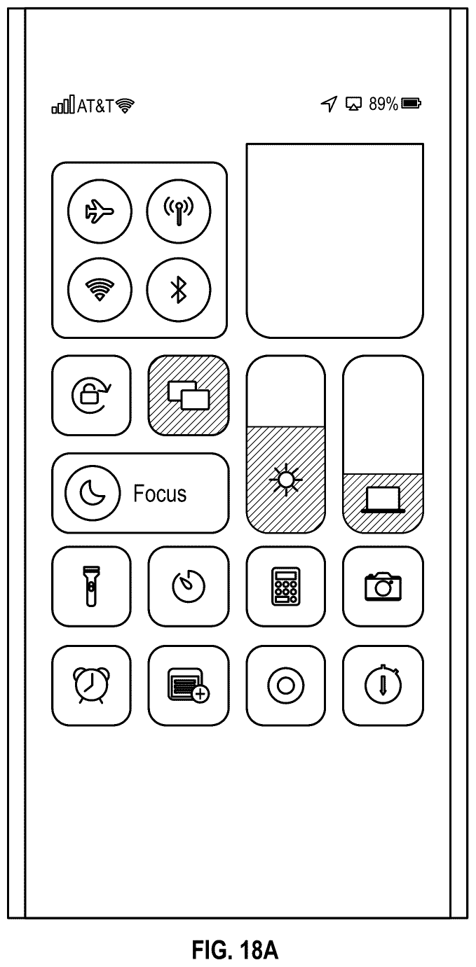

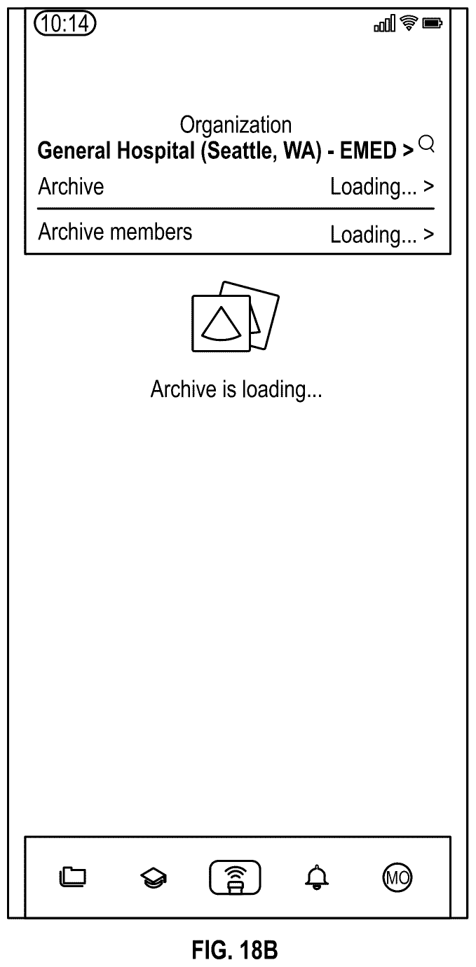

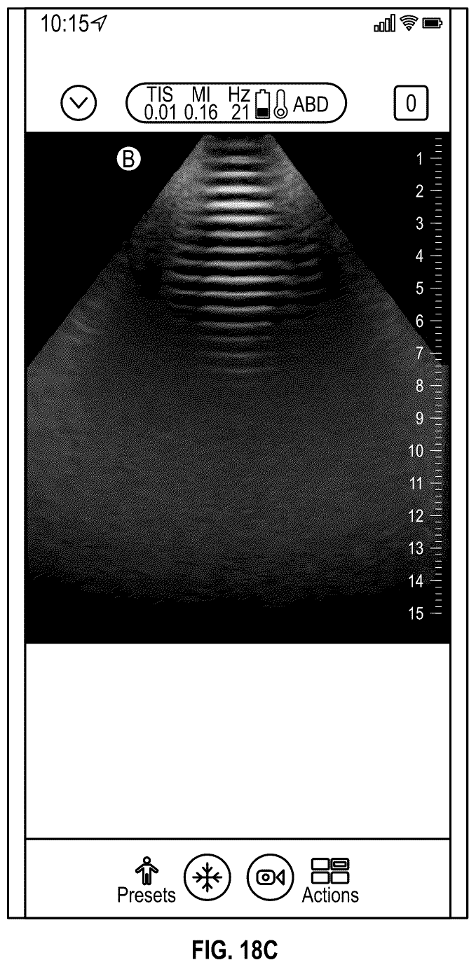

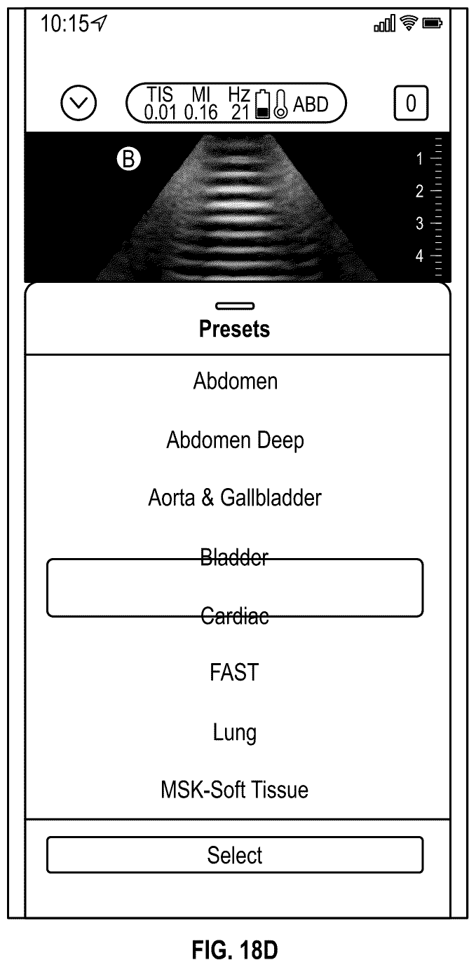

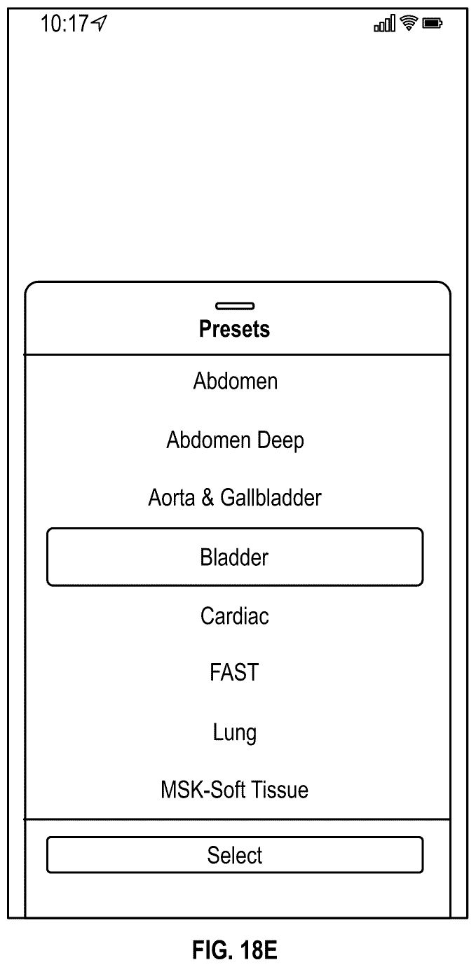

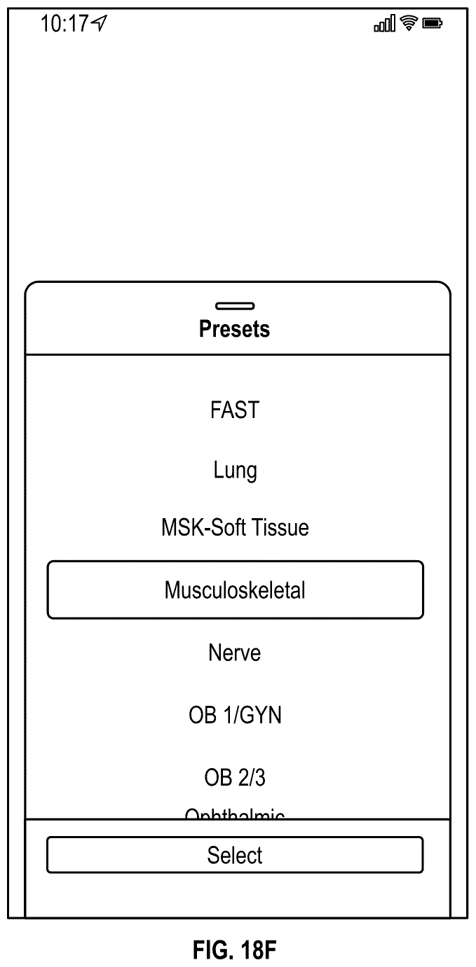

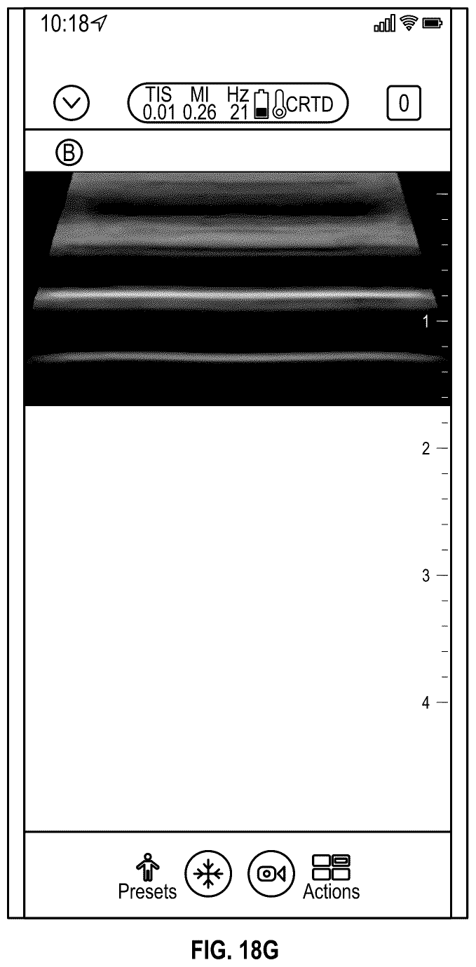

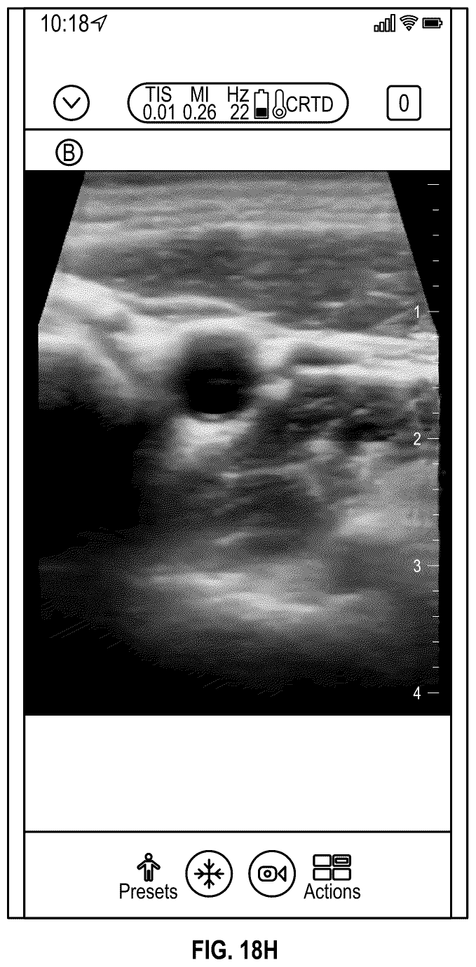

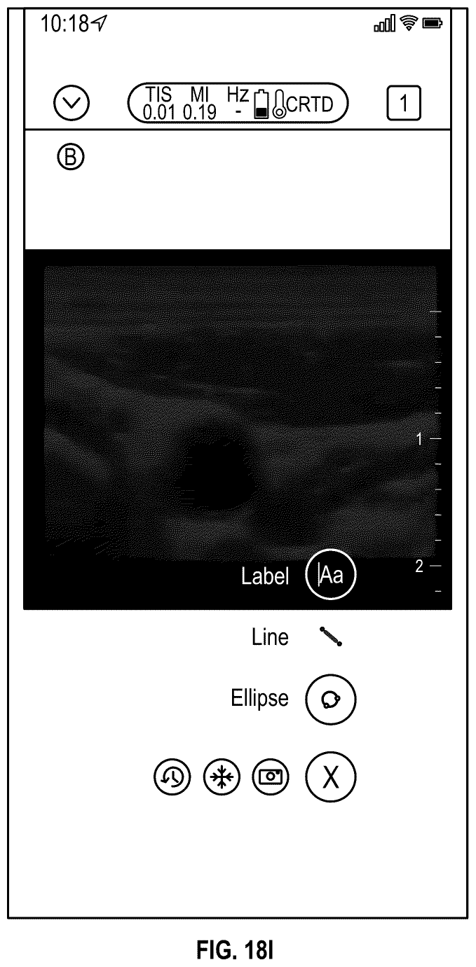

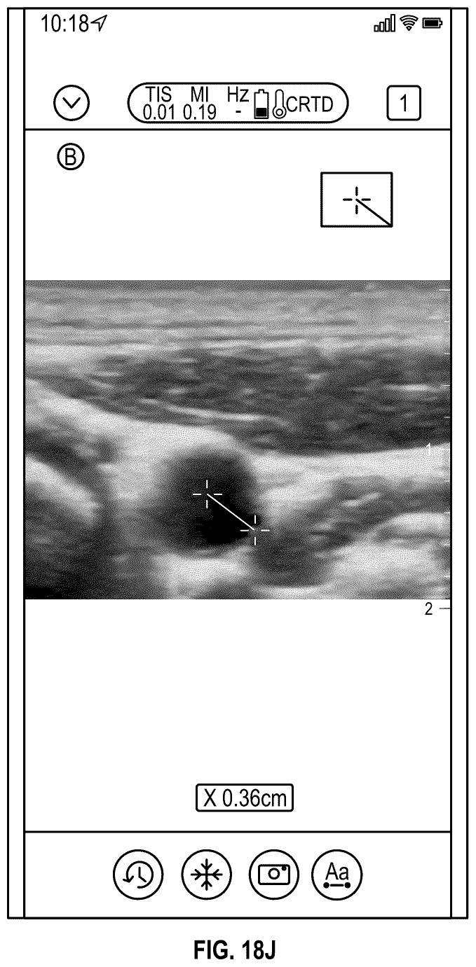

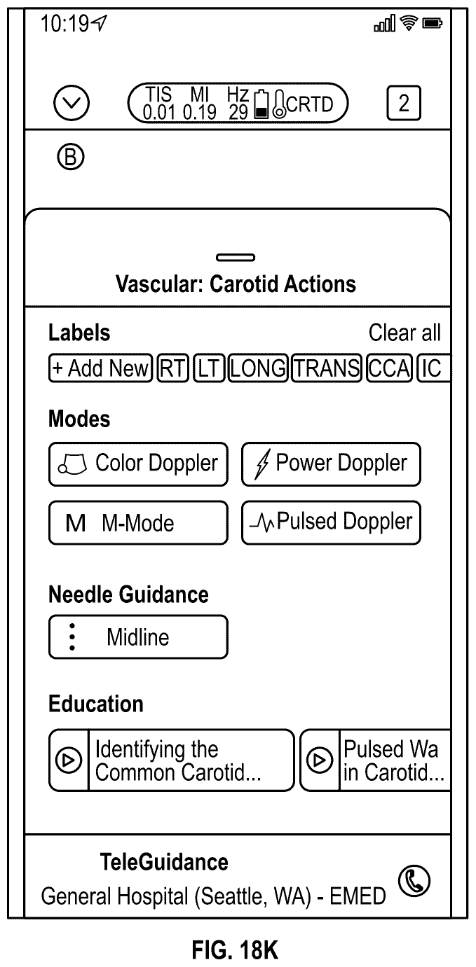

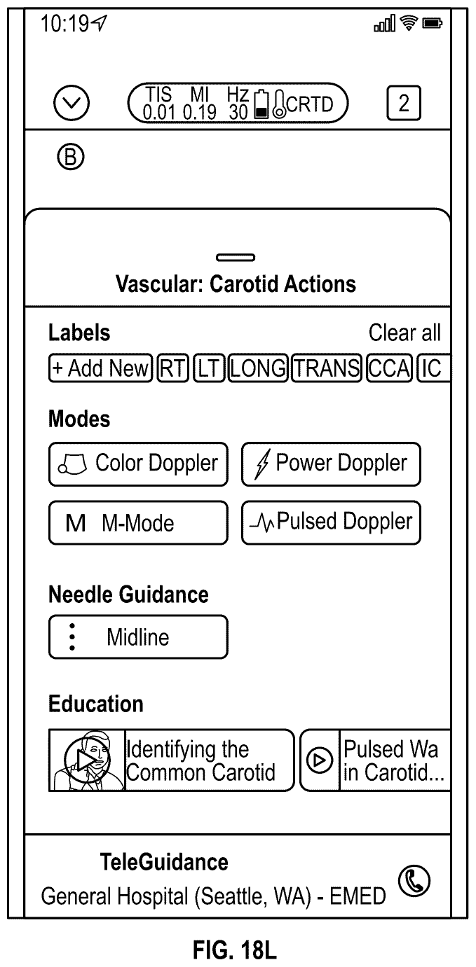

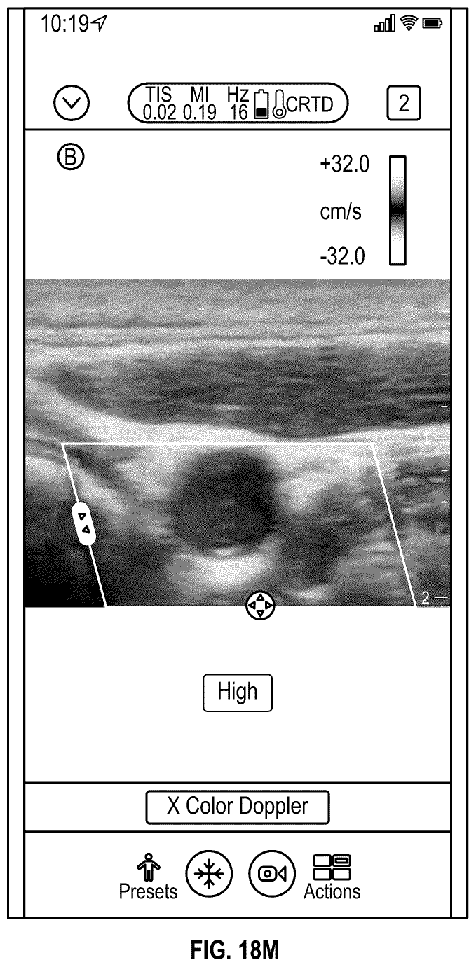

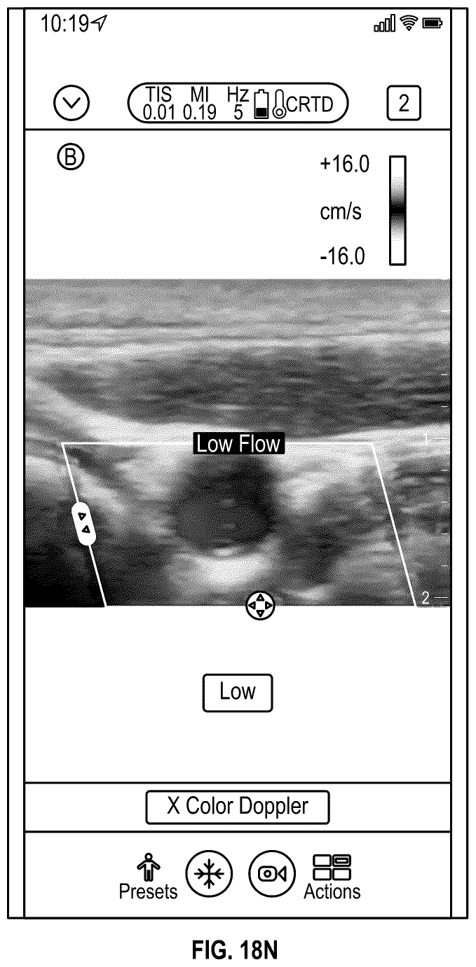

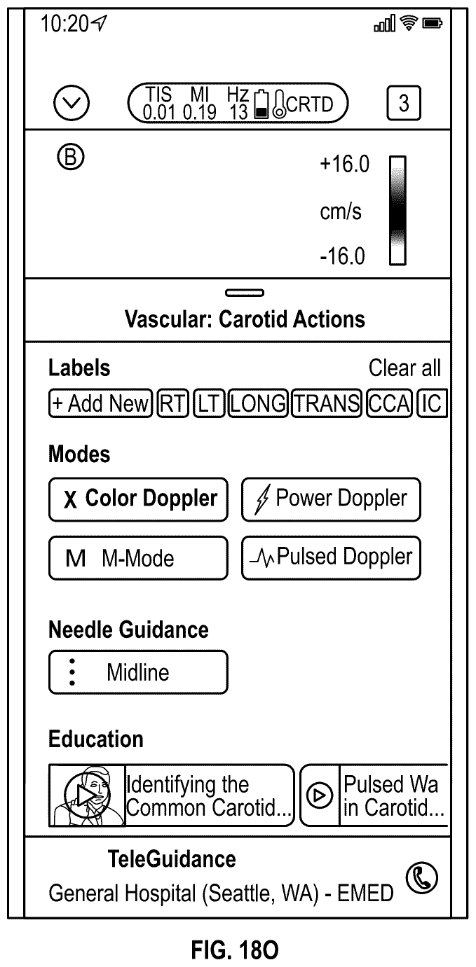

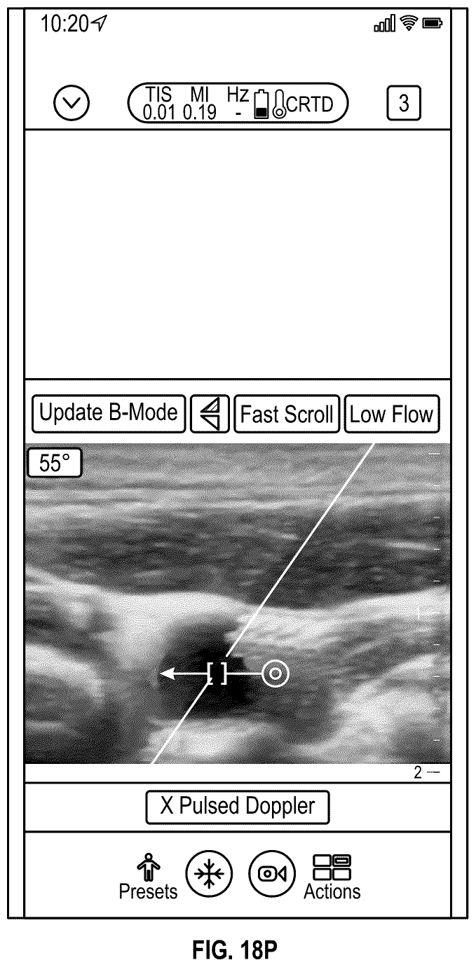

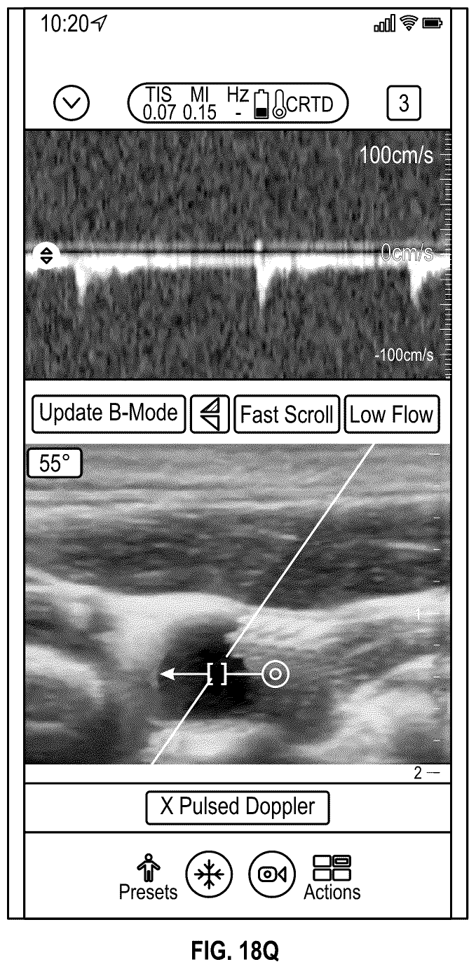

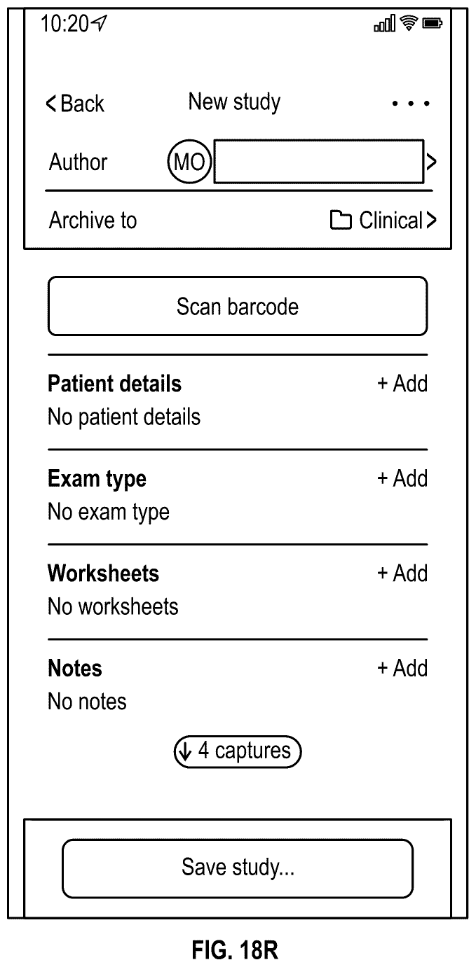

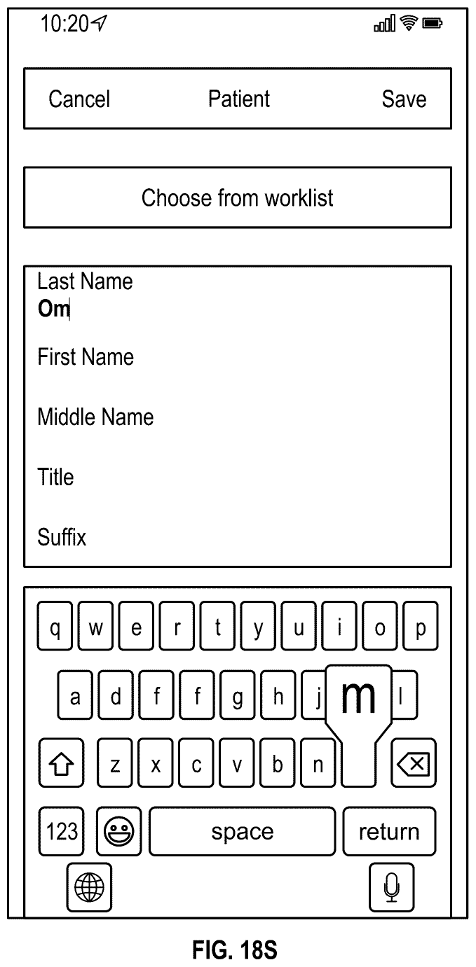

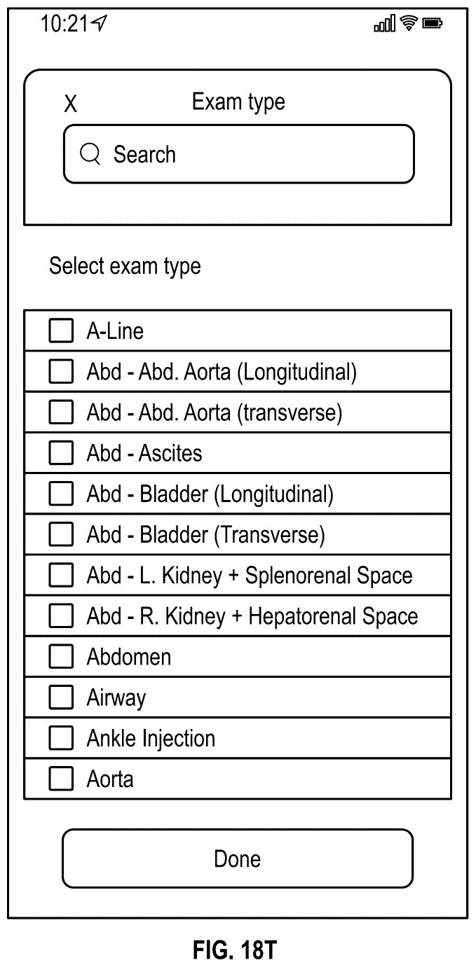

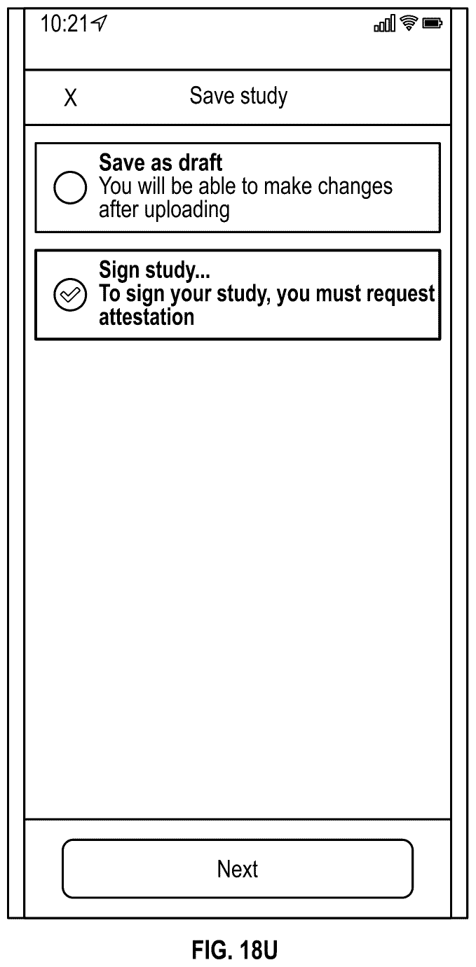

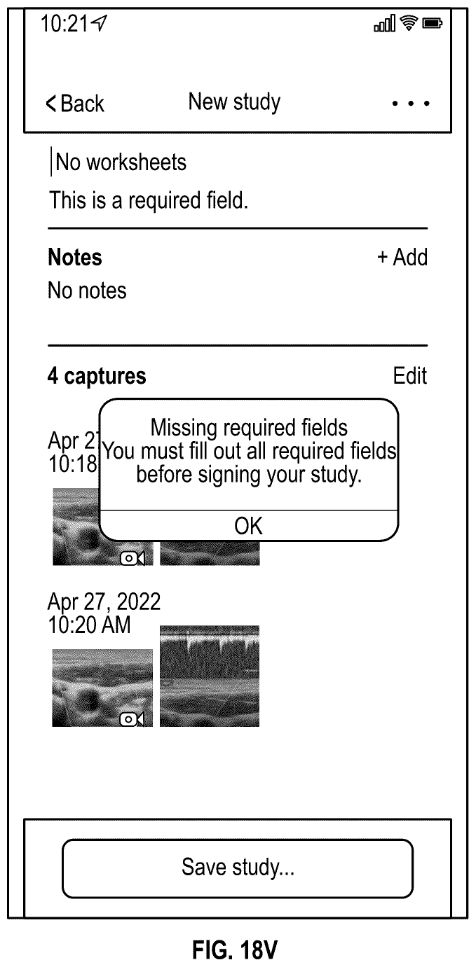

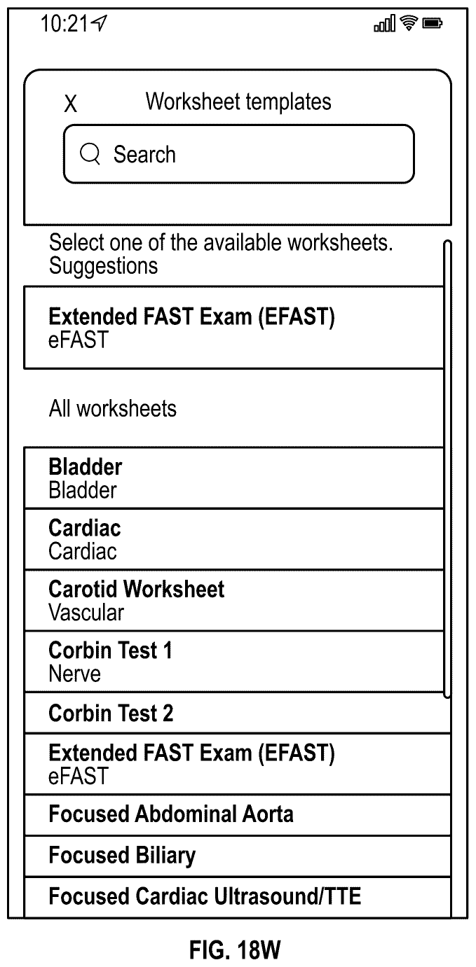

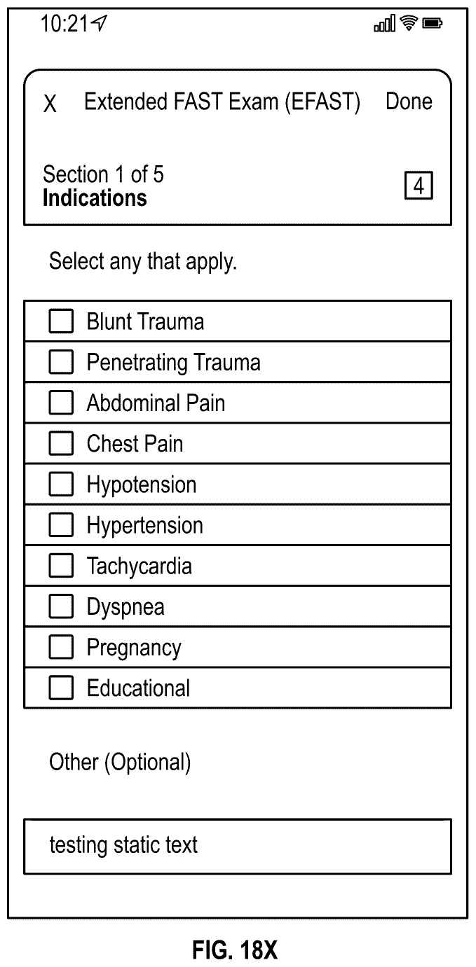

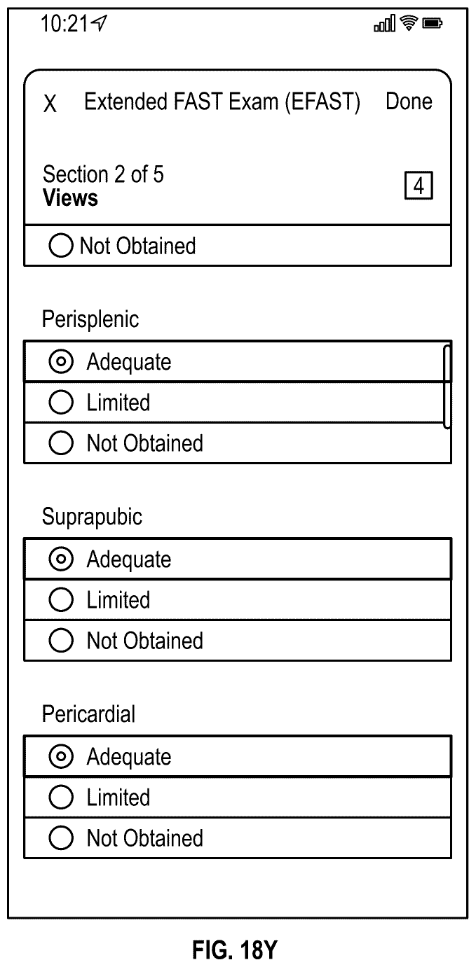

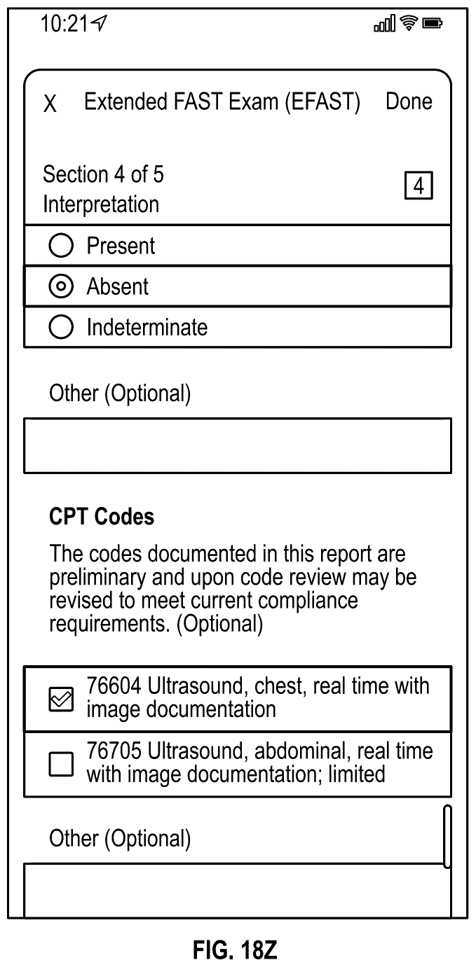

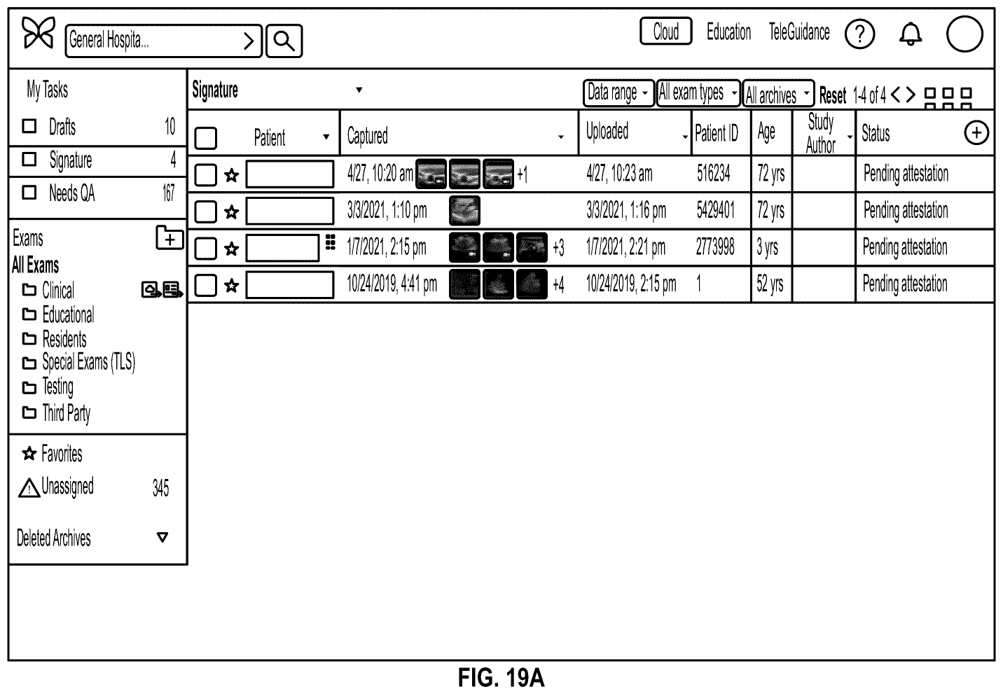

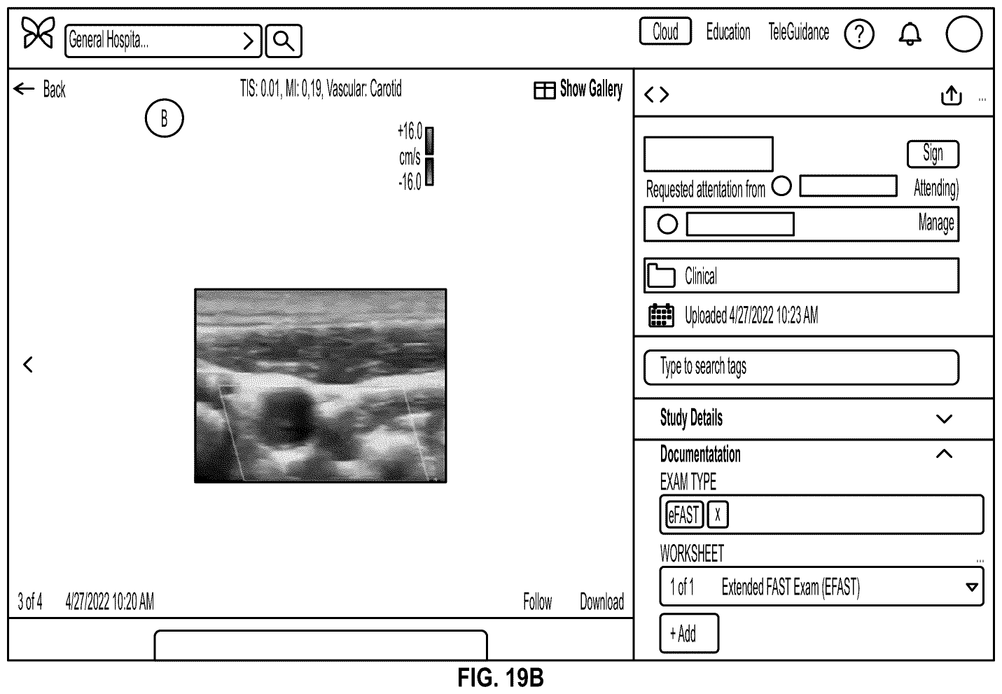

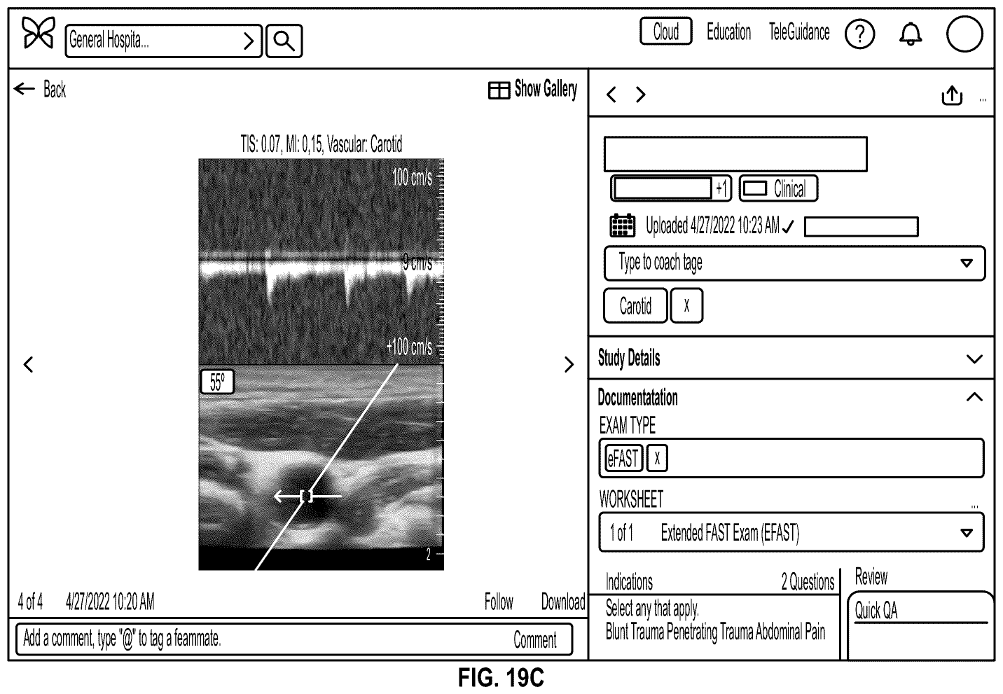

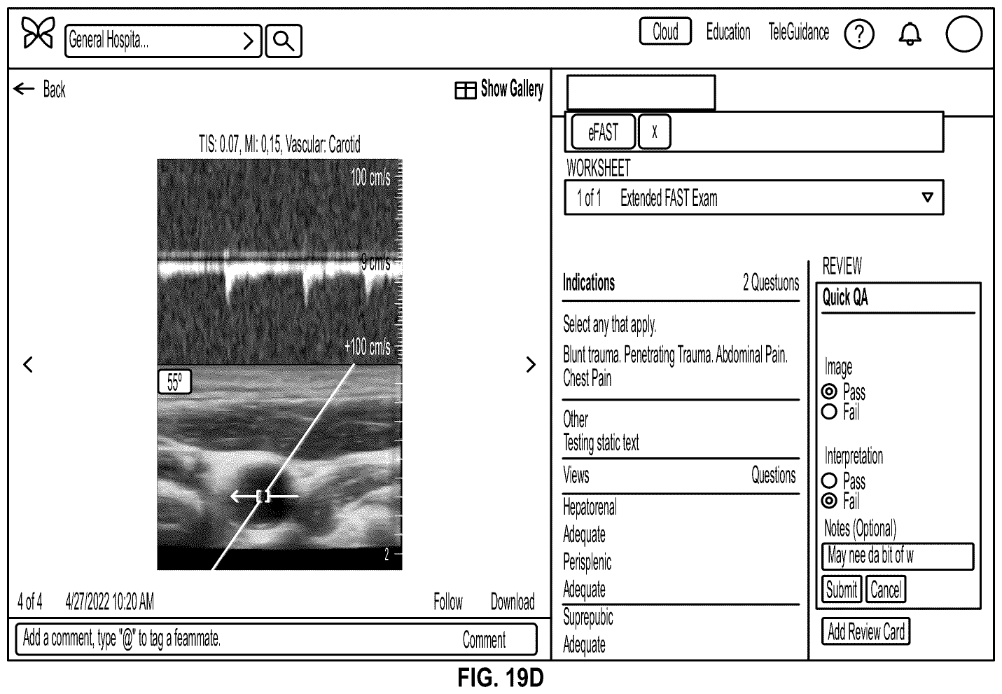

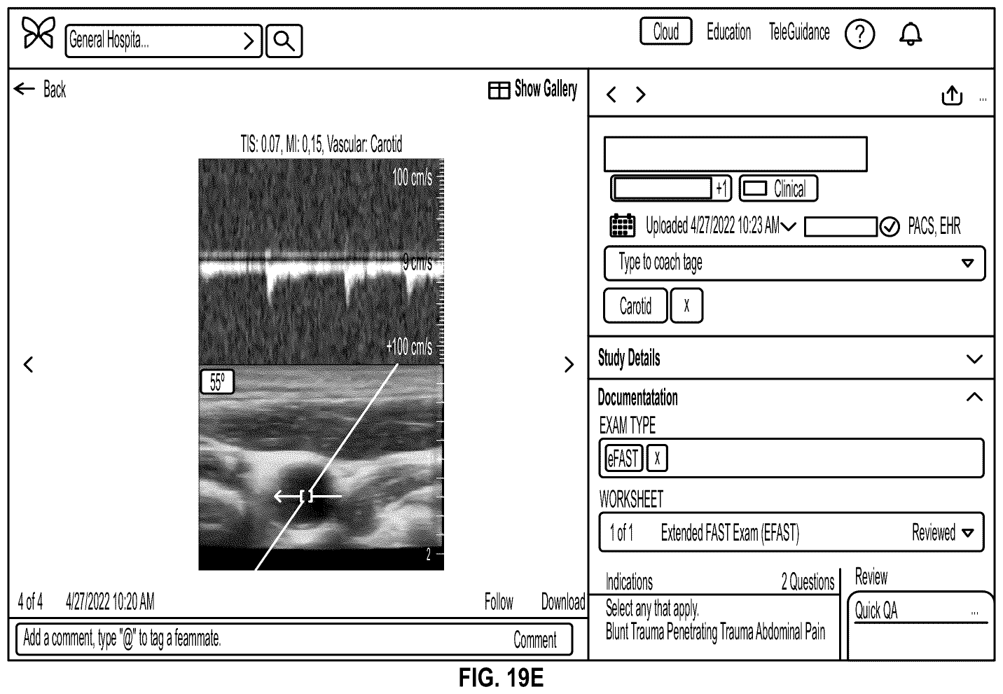

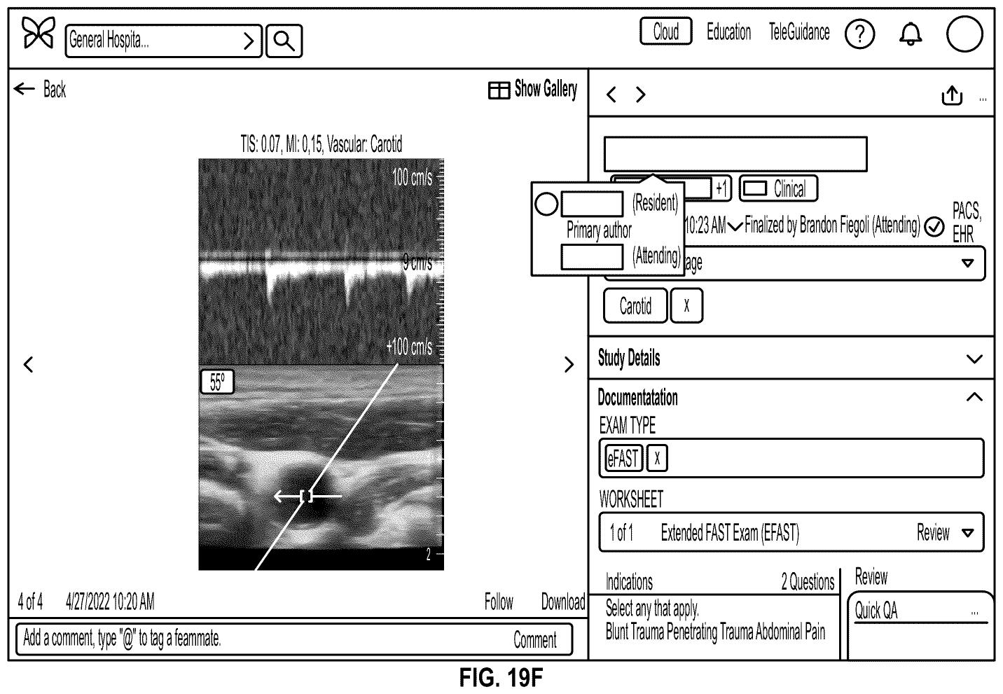

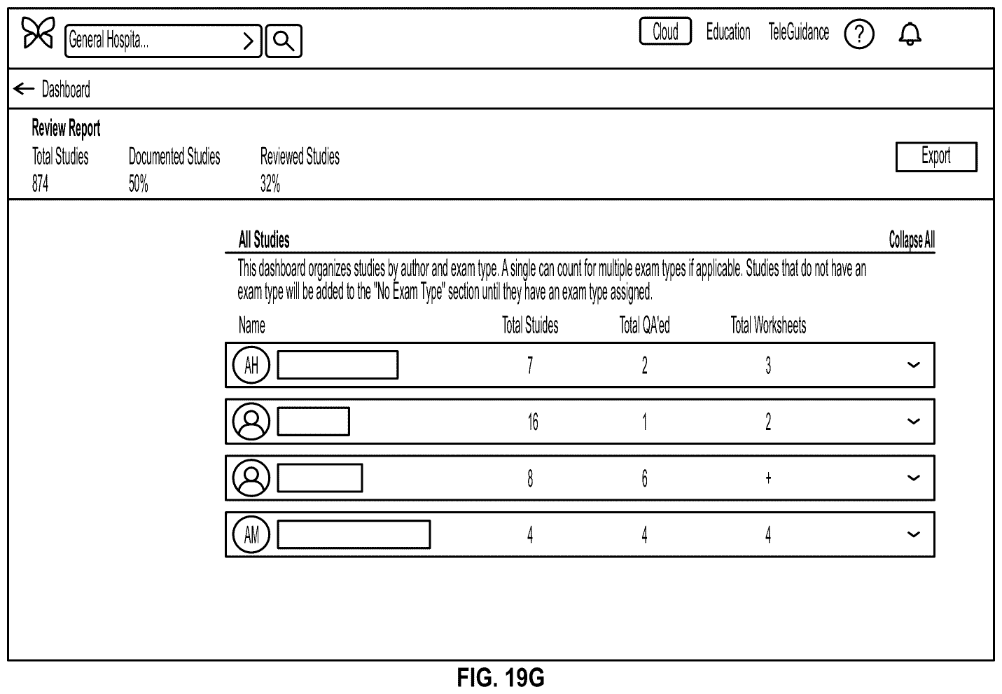

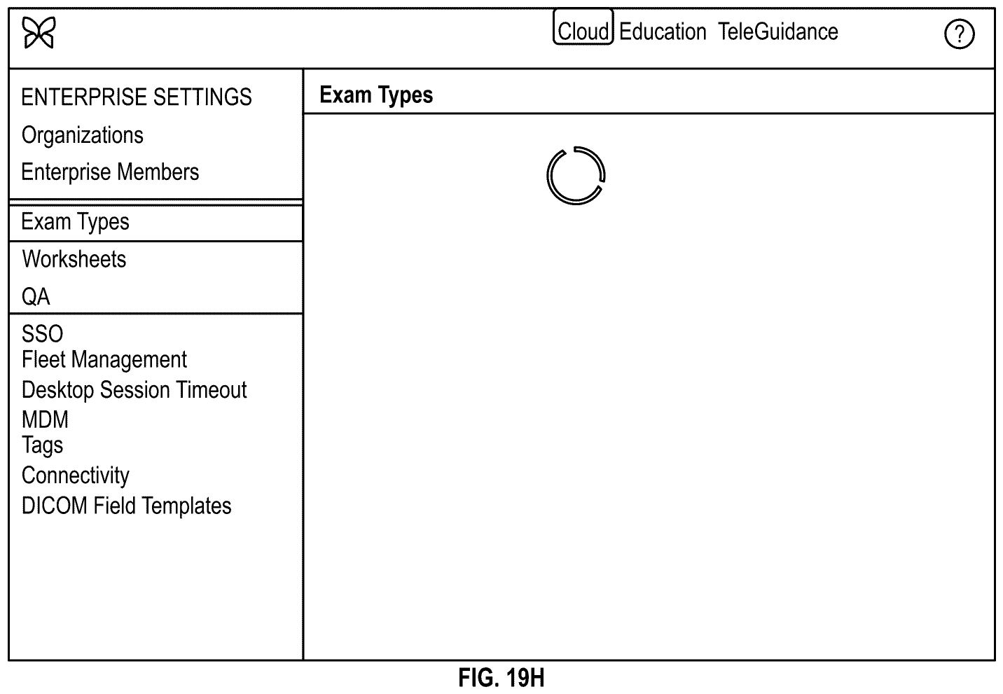

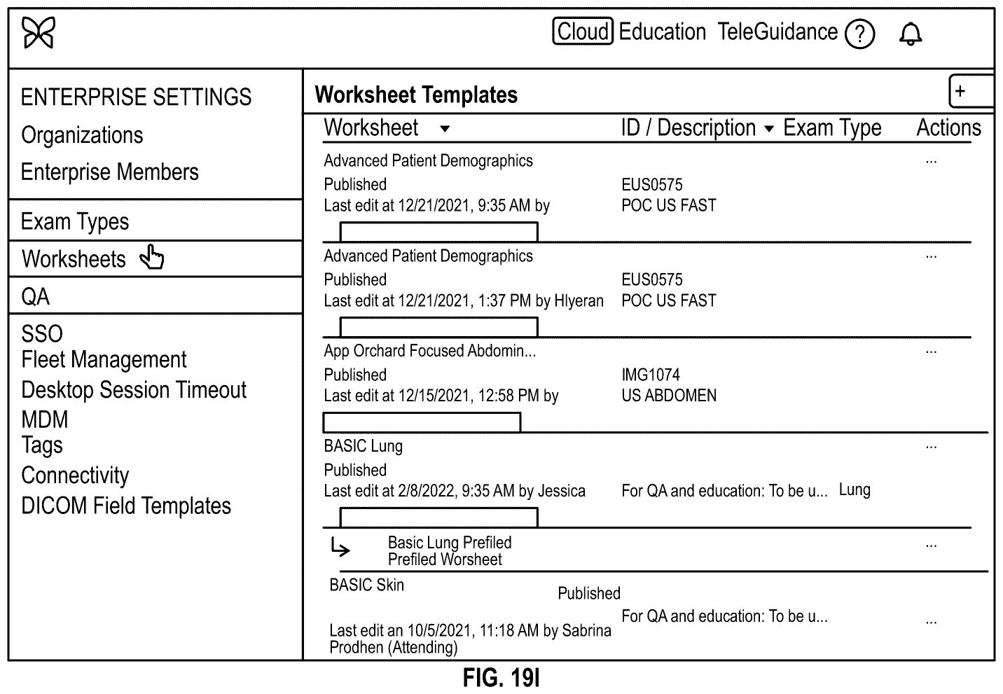

Turning to FIGS. 18A-18Z and 19A-19I, 18A-18Z and 19A-19I show examples of graphical user interfaces in accordance with one or more embodiments. For example, a user may interact with a graphical user interface (GUI) on a processing device at one or more steps in an ultrasound imaging examination (e.g., automatically initiating an ultrasound application on the processing device, automatically determining a patient, organization, a mode, a preset, a TGC parameter, an imaging depth, etc.). Accordingly, one or more non-GUI inputs (e.g., voice commands, voice responses, inputs from artificial intelligence processes, etc.) may be provide during operation of the processing device at one or more GUI screens shown in FIGS. 18A-18Z and 19A-19I, 18A-18Z and 19A-19I.

In some embodiments, an ultrasound system for performing an ultrasound imaging exam includes an ultrasound imaging device; and a processing device in operative communication with the ultrasound imaging device and configured to perform a method. The method may include initiating an ultrasound imaging application. The method may include receiving a selection of one or more user credentials. The method may include automatically selecting an organization or receive a voice command from a user to select the organization. The method may include automatically selecting a patient or receive a voice command from the user to select the patient. The method may include automatically determining whether a sufficient amount of gel has been applied to the ultrasound imaging device, and upon determining that the sufficient amount of gel has not been applied to the ultrasound imaging device, provide an instruction to the user to apply more gel to the ultrasound imaging device. The method may include automatically selecting or receives a selection of an ultrasound imaging exam type. The method may include automatically select an ultrasound imaging mode or receive a voice command from the user to select the ultrasound imaging mode. The method may include automatically selecting an ultrasound imaging preset or receive a voice command from the user to select the ultrasound imaging preset. The method may include automatically selecting an ultrasound imaging depth or receive a voice command from the user to select the ultrasound imaging depth. The method may include automatically select an ultrasound imaging gain or receive a voice command from the user to select the ultrasound imaging gain. The method may include automatically selecting one or more time gain compensation (TGC) parameters or receive a voice command from the user to select the one or more TGC parameters. The method may include guiding the user to correctly place the ultrasound imaging device in order to capture one or more clinically relevant ultrasound images. The method may include automatically capturing or receive a voice command to capture the one or more clinically relevant ultrasound images. The method may include automatically completing a portion or all of an ultrasound imaging worksheet or receive a voice command from the user to complete the portion or all of the ultrasound imaging worksheet. The method may include associating a signature with the ultrasound imaging exam or request signature of the ultrasound imaging exam later. The method may include automatically uploading the ultrasound imaging exam or receive a voice command from the user to upload the ultrasound imaging exam.

In some embodiments, a processing device initiates the ultrasound imaging application in response to: the user connecting the ultrasound imaging device into the processing device; the ultrasound imaging device being brought into proximity of the processing device; the user pressing a button of the ultrasound imaging device; or the user providing a voice command. In some embodiments, a processing device is configured to automatically select the patient by: receiving a scan of a barcode associated with the patient; performing facial recognition of the patient; performing fingerprint recognition of the patient; performing voice recognition of the patient: receiving an image of a medical chart associated with the patient; or retrieving a calendar of the user and selecting the patient based on the calendar. In some embodiments, a processing device is configured to automatically select the organization by: selecting a default organization associated with the user; selecting a default organization associated with the ultrasound imaging device; or selecting the organization based on a global positioning system (GPS) in the processing device or the ultrasound imaging device. In some embodiments, a processing device is configured to automatically select the ultrasound imaging preset by: selecting a default ultrasound imaging preset associated with the user; selecting a default ultrasound imaging preset associated with the ultrasound imaging device; retrieving an electronic medical record (EMR) of the patient and selecting the ultrasound imaging preset based on the EMR; retrieving a calendar of the user and selecting the ultrasound imaging preset based on the calendar. In some embodiments, a processing device is configured to automatically select an ultrasound imaging exam type by: retrieving a calendar of the user and selecting the ultrasound imaging exam type based on the calendar; or analyzing the one or more clinically relevant ultrasound images using artificial intelligence. In some embodiments, a processing device is configured to automatically complete the portion or all of the ultrasound imaging worksheet by: retrieving an electronic medical record (EMR) of the patient and completing the portion or all of the ultrasound imaging worksheet based on the EMR, and/or providing an audio prompt to the user. In some embodiments, a processing device is configured to associate the signature with the ultrasound imaging exam based on: a voice command from the user; facial recognition of the user; fingerprint recognition of the user; or voice recognition of the user.

In some embodiments, an ultrasound system for performing an ultrasound imaging exam include an ultrasound imaging device; and a processing device in operative communication with the ultrasound imaging device and configured to perform a method. The method may include automatically selecting a patient or receive a selection of the patient from a user. The method may include automatically selecting an ultrasound imaging exam type or receive a selection from the user of the ultrasound imaging exam type. The method may include automatically selecting an ultrasound imaging mode, an ultrasound imaging preset, an ultrasound imaging depth, an ultrasound imaging gain, and/or one or more time gain compensation (TGC) parameters corresponding to the ultrasound imaging exam type. The method may include guiding a user to correctly place the ultrasound imaging device on the patient for capturing one or more ultrasound images associated with a first scan of the ultrasound imaging exam by using one or more of: one or more images, one or more videos, audio, and/or text that indicate how to place the ultrasound imaging device on the patient; a real-time quality indicator indicating a quality of recent ultrasound data collected by the ultrasound imaging device; and automatic anatomical and/or pathological labeling of one or more ultrasound images captured by ultrasound imaging device. The method may include capturing one or more ultrasound images associated with the first scan of the ultrasound imaging exam by: automatically capturing a multi-second cine of ultrasound images in response to the quality of the recent ultrasound data exceeding a first threshold; or receiving a command from the user to capture the one or more ultrasound images. The method may include automatically advancing to guide the user to correctly place the ultrasound imaging device on the patient for capturing one or more ultrasound images associated with a second scan of the ultrasound imaging exam. The method may include providing a summary of the ultrasound imaging exam. The method may include providing an option for the user to review the captured one or more ultrasound images.

In some embodiments, an ultrasound imaging exam type is an exam assessing heart and lung function. In some embodiments, a processing device is configured to automatically select the exam assessing heart and lung function for all patients. In some embodiments, a first scan of the ultrasound imaging exam comprises capturing one or more ultrasound images of an anterior-superior view of a right lung, a lateral-superior view of the right lung, a lateral-inferior view of the right lung, an anterior-superior view of a left lung, a lateral-superior view of the left lung, a lateral-inferior view of the left lung, a parasternal long axis view of a heart, or an apical four chamber view of the heart. In some embodiments, a first scan of the ultrasound imaging exam comprises capturing one or more ultrasound images of a lung and the second scan of the ultrasound imaging exam comprises capturing one or more ultrasound images of a heart. In some embodiments, an automatic anatomical and/or pathological labeling comprises labeling A lines, B lines, a pleural line, a right ventricle, a left ventricle, a right atrium, and/or a left atrium. In some embodiments, a processing device is further configured to disable capturing the one or more ultrasound images associated with the first scan of the ultrasound imaging exam when the quality of the recent ultrasound data does not exceed a second threshold. In some embodiments, a processing device is configured, when providing the summary of the ultrasound imaging exam, to provide a single score for the ultrasound imaging exam. In some embodiments, a single score is based on one or more of: a number of scans completed; whether or not a plurality of scans are auto-captured, or if the plurality of scans are manually captured, an average quality score for the plurality of scans; and which of a plurality of automatic calculations are calculated. In some embodiments, a processing device is configured, when providing the summary of the ultrasound imaging exam, to provide a count of scans automatically captured and a count of scans missing. In some embodiments, a method further includes automatically calculating and displaying: a left ventricular diameter, a left atrial diameter, a right ventricular diameter, a right atrial diameter, and an ejection fraction based on an apical four chamber scan; the left ventricular diameter, the left atrial diameter, and the right ventricular diameter based on a parasternal long axis scan; and a number of B lines based on each of a plurality of lung scans. In some embodiments, a processing device is further configured to display progress through a plurality of scans of the ultrasound imaging exam. In some embodiments, automatically capturing the multi-second cine of ultrasound images includes: capturing a six-second cine of ultrasound images of a lung; and capturing a three-second cine of ultrasound images of a heart. In some embodiments, automatic anatomical and/or pathological labeling includes labeling A lines, B lines, a pleural line, a right ventricle, a left ventricle, a right atrium, and/or a left atrium. In some embodiments, a processing device is further configured to disable capturing the one or more ultrasound images associated with the first scan of the ultrasound imaging exam when the quality of the recent ultrasound data does not exceed a second threshold.

In some embodiments, a processing device is configured, when providing the summary of the ultrasound imaging exam, to provide a single score for the ultrasound imaging exam. In some embodiments, a single score is based on one or more of: a number of scans completed; whether or not a plurality of scans are auto-captured, or if the plurality of scans are manually captured, an average quality score for the plurality of scans; and which of a plurality of automatic calculations are calculated. In some embodiments, a processing device is configured, when providing the summary of the ultrasound imaging exam, to provide a count of scans automatically captured and a count of scans missing. In some embodiments, automatically calculating and displaying includes displaying a left ventricular diameter, a left atrial diameter, a right ventricular diameter, a right atrial diameter, and an ejection fraction based on an apical four chamber scan; the left ventricular diameter, the left atrial diameter, and the right ventricular diameter based on a parasternal long axis scan; and a number of B lines based on each of a plurality of lung scans. In some embodiments, a processing device is further configured to display progress through a plurality of scans of the ultrasound imaging exam. In some embodiments, automatically capturing the multi-second cine of ultrasound images includes: capturing a six-second cine of ultrasound images of a lung; and capturing a three-second cine of ultrasound images of a heart. In some embodiments, a processing device is configured, when capturing the one or more ultrasound images associated with the first scan of the ultrasound imaging exam, to monitor a quality of the captured one or more ultrasound images and stop the capture if the quality is below a threshold quality. In some embodiments, an ultrasound imaging exam type is an exam performed on a patient with congestive heart failure to monitor the patient for pulmonary edema.

Although only a few example embodiments have been described in detail above, those skilled in the art will readily appreciate that many modifications are possible in the example embodiments without materially departing from this invention. Accordingly, all such modifications are intended to be included within the scope of this disclosure as defined in the following claims.

What is claimed is:

1. An ultrasound system for performing an ultrasound imaging exam, comprising:
an ultrasound imaging device; and
a processing device in operative communication with the ultrasound imaging device and configured to:
initiate an ultrasound imaging application;
receive a selection of one or more user credentials;
automatically select an organization or receive a voice command from a user to select the organization;
automatically select a patient or receive a voice command from the user to select the patient;
automatically determine whether a sufficient amount of gel has been applied to the ultrasound imaging device, and upon determining that the sufficient amount of gel has not been applied to the ultrasound imaging device, provide an instruction to the user to apply more gel to the ultrasound imaging device;
automatically select or receives a selection of an ultrasound imaging exam type;
automatically select an ultrasound imaging mode or receive a voice command from the user to select the ultrasound imaging mode;
automatically select an ultrasound imaging preset or receive a voice command from the user to select the ultrasound imaging preset;
automatically select an ultrasound imaging depth or receive a voice command from the user to select the ultrasound imaging depth;
automatically select an ultrasound imaging gain or receive a voice command from the user to select the ultrasound imaging gain;
automatically select one or more time gain compensation (TGC) parameters or receive a voice command from the user to select the one or more TGC parameters;
guide the user to correctly place the ultrasound imaging device in order to capture one or more clinically relevant ultrasound images;
automatically capture or receive a voice command to capture the one or more clinically relevant ultrasound images;
automatically complete a portion or all of an ultrasound imaging worksheet or receive a voice command from the user to complete the portion or all of the ultrasound imaging worksheet;
associate a signature with the ultrasound imaging exam or request signature of the ultrasound imaging exam later; and
automatically upload the ultrasound imaging exam or receive a voice command from the user to upload the ultrasound imaging exam.

2. The ultrasound system of claim 1, wherein the processing device is configured to initiate the ultrasound imaging application in response to:
the user connecting the ultrasound imaging device into the processing device;
the ultrasound imaging device being brought into proximity of the processing device;
the user pressing a button of the ultrasound imaging device; or
the user providing a voice command.

3. The ultrasound system of claim 1, wherein the processing device is configured to automatically select the patient by:
receiving a scan of a barcode associated with the patient;
performing facial recognition of the patient;
performing fingerprint recognition of the patient;
performing voice recognition of the patient:
receiving an image of a medical chart associated with the patient; or
retrieving a calendar of the user and selecting the patient based on the calendar.

4. The ultrasound system of claim 1, wherein the processing device is configured to automatically select the organization by:
selecting a default organization associated with the user;
selecting a default organization associated with the ultrasound imaging device; or
selecting the organization based on a global positioning system (GPS) in the processing device or the ultrasound imaging device.

5. The ultrasound system of claim 1, wherein the processing device is configured to automatically select the ultrasound imaging preset by:
selecting a default ultrasound imaging preset associated with the user;
selecting a default ultrasound imaging preset associated with the ultrasound imaging device;
retrieving an electronic medical record (EMR) of the patient and selecting the ultrasound imaging preset based on the EMR;
retrieving a calendar of the user and selecting the ultrasound imaging preset based on the calendar.

6. The ultrasound system of claim 1, wherein the processing device is configured to automatically select an ultrasound imaging exam type by:
retrieving a calendar of the user and selecting the ultrasound imaging exam type based on the calendar; or
analyzing the one or more clinically relevant ultrasound images using artificial intelligence.

7. The ultrasound system of claim 1, wherein the processing device is configured to automatically complete the portion or all of the ultrasound imaging worksheet by:
retrieving an electronic medical record (EMR) of the patient and completing the portion or all of the ultrasound imaging worksheet based on the EMR, and/or providing an audio prompt to the user.

8. The ultrasound system of claim 1, wherein the processing device is configured to associate the signature with the ultrasound imaging exam based on:
a voice command from the user;
facial recognition of the user;
fingerprint recognition of the user; or
voice recognition of the user.

9. An ultrasound system for performing an ultrasound imaging exam, comprising:
an ultrasound imaging device; and
a processing device in operative communication with the ultrasound imaging device and configured to:
automatically select a patient or receive a selection of the patient from a user;
automatically select an ultrasound imaging exam type or receive a selection from the user of the ultrasound imaging exam type;
automatically selects an ultrasound imaging mode, an ultrasound imaging preset, an ultrasound imaging depth, an ultrasound imaging gain, and/or one or more time gain compensation (TGC) parameters corresponding to the ultrasound imaging exam type;
guide a user to correctly place the ultrasound imaging device on the patient for capturing one or more ultrasound images associated with a first scan of the ultrasound imaging exam by using one or more of:
one or more images, one or more videos, audio, and/or text that indicate how to place the ultrasound imaging device on the patient;
a real-time quality indicator indicating a quality of recent ultrasound data collected by the ultrasound imaging device; and
automatic anatomical and/or pathological labeling of one or more ultrasound images captured by ultrasound imaging device;
capture one or more ultrasound images associated with the first scan of the ultrasound imaging exam by:
automatically capturing a multi-second cine of ultrasound images in response to the quality of the recent ultrasound data exceeding a first threshold; or
receiving a command from the user to capture the one or more ultrasound images;
automatically advancing to guide the user to correctly place the ultrasound imaging device on the patient for capturing one or more ultrasound images associated with a second scan of the ultrasound imaging exam;
provide a summary of the ultrasound imaging exam; and
provide an option for the user to review the captured one or more ultrasound images.

10. The ultrasound system of claim 9, wherein the ultrasound imaging exam type is an exam assessing heart and lung function.

11. The ultrasound system of claim 10, wherein the processing device is configured to automatically select the exam assessing heart and lung function for all patients.

12. The ultrasound system of claim 10, wherein the first scan of the ultrasound imaging exam comprises capturing one or more ultrasound images of an anterior-superior view of a right lung, a lateral-superior view of the right lung, a lateral-inferior view of the right lung, an anterior-superior view of a left lung, a lateral-superior view of the left lung, a lateral-inferior view of the left lung, a parasternal long axis view of a heart, or an apical four chamber view of the heart.

13. The ultrasound system of claim 10, wherein the first scan of the ultrasound imaging exam comprises capturing one or more ultrasound images of a lung and the second scan of the ultrasound imaging exam comprises capturing one or more ultrasound images of a heart.

14. The ultrasound system of claim 10, wherein the automatic anatomical and/or pathological labeling comprises labeling A lines, B lines, a pleural line, a right ventricle, a left ventricle, a right atrium, and/or a left atrium.

15. The ultrasound system of claim 9, wherein the processing device is further configured to disable capturing the one or more ultrasound images associated with the first scan of the ultrasound imaging exam when the quality of the recent ultrasound data does not exceed a second threshold.

16. The ultrasound system of claim 9, wherein the processing device is configured, when providing the summary of the ultrasound imaging exam, to provide a single score for the ultrasound imaging exam.

17. The ultrasound system of claim 16, wherein the single score is based on one or more of:
a number of scans completed;
whether or not a plurality of scans are auto-captured, or if the plurality of scans are manually captured, an average quality score for the plurality of scans; and
which of a plurality of automatic calculations are calculated.

18. The ultrasound system of claim 9, wherein the processing device is configured, when providing the summary of the ultrasound imaging exam, to provide a count of scans automatically captured and a count of scans missing.

19. The ultrasound system of claim 10, further comprising automatically calculating and displaying:
a left ventricular diameter, a left atrial diameter, a right ventricular diameter, a right atrial diameter, and an ejection fraction based on an apical four chamber scan;
the left ventricular diameter, the left atrial diameter, and the right ventricular diameter based on a parasternal long axis scan; and
a number of B lines based on each of a plurality of lung scans.

20. The ultrasound system of claim 9, wherein the processing device is further configured to display progress through a plurality of scans of the ultrasound imaging exam.
